(19) United States
(12) Patent Application Publication
(10) Pub. No.: US 2026/0152778 A1
(43) Pub. Date: Jun. 4, 2026

(54) CELL-FREE PROTEIN SYNTHESIS SYSTEMS, REACTION MIXTURES FOR SAME, AND METHODS FOR PREPARING PROTEINS

(71) Applicant: SANOFI PASTEUR INC., Toronto (CA)

(72) Inventors: Athanasios Evangelos KRITHARIS, Vancouver (CA); Michaela SAMANTA, Vancouver (CA); Ibrahim Melih TAMER, Cambridge, MA (US); Vikramaditya Ganapati YADAV, Vancouver (CA)

(21) Appl. No.: 19/123,272

(22) PCT Filed: Oct. 24, 2023
(86) PCT No.: PCT/CA2023/051409

§ 371 Date: Apr. 22, 2025

(30) Foreign Application Priority Data
Oct. 24, 2022 (EP) 22203404.3

(51) Int. Cl. C12P 21/00

(52) U.S. Cl. CPC C12P 21/00

(57) ABSTRACT

Disclosed herein are cell-free protein synthesis systems, reaction mixtures for the same, and methods for preparing a protein of interest.

### INTRODUCTION AND SUMMARY

Cell-free protein synthesis (CFPS) offers many advantages over cell-based protein synthesis, such as time savings and increased overall yields of functional, soluble, full-length proteins. Additionally, CFPS is not as sensitive to toxic proteins and is adaptable to high-throughput experiments.

Commercial CFPS mixtures typically requires complex and costly feedstock such as free amino acids, free ribonucleotides, and free NADH/NADPH. Although commercial CFPS typically emphasizes reaction speed, its yield is often restricted by the feedstock being a rate limiting step, as feedstock has to be produced and regenerated before it can be converted into energy and/or protein products. Additionally, commercial CFPS often relies on systems that need to be preserved at lower temperatures, such as −20° C.

In contrast, the present application discloses novel CFPS systems, including their novel reaction mixtures, that use simpler, less expensive feedstock and that avoid many of the issues facing commercial CFPS (e.g. wasteful consumption of amino acids in side-reactions, regeneration of NADH/NADPH) and provides the benefit of reduced cost. Additionally, the novel CFPS systems and reaction mixtures may be stored at 4° C. and/or room temperature, thus avoiding the inconvenience and cost associated with storage at lower temperatures.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows the effects of extract and mixture conditions, as well as temperature, on CFPS deGFP protein yield. “Min” and “Com” refer to “minimal” and “commercial,” respectively, and “CE” refers to cell extract.

FIG. 2 shows the effect of removing individual reaction mixture components on CFPS deGFP protein yield when utilizing a minimal cell extract.

FIG. 3 shows the effect of sodium chloride concentration on CFPS protein yield when utilizing minimal cell extract from V. natriegens.

### DETAILED DESCRIPTION OF THE DISCLOSURE

### Definitions

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. To the extent any material incorporated herein by reference is inconsistent with the express content of this disclosure, the express content controls. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the inventions.

As used herein, ranges and amounts can be expressed as “about” a particular value or range. “About” also includes the exact amount. Hence “about 5 μL” means “about 5 μL” and also “5 μL.” Generally, the term “about” includes an amount that would be expected to be within experimental error. For example, the term

The section headings used herein are for organizational purposes only and are not to be construed as limiting the subject matter described.

As used herein, a “cell-free,” or “CF” system or element thereof is a system or element that performs its intended function (e.g., protein systhensis) by predominantly utilizing non-live-cell aspects (e.g., supernatant collected from lysed bacteria) of the system or element. A “cell-free” system or element thereof may contain residual amounts of live cells.

As used herein, “minimal media” is media that contains only the minimum set of nutrients required for bacterial growth. Said nutrients may vary according to the bacterial species, and will be known to the skilled person for a given species. In an embodiment, “Minimal media” substantially consists of (a) at least one carbon source (e.g., a sugar such as glucose, a polyol such as glycerol, or a dicarboxylic acid such as succinate), (b) at least one of an ammonium source (e.g., ammonium sulphate), a phosphate source (e.g., monopotassium phosphate or dipotassium phosphate), a sulfur source, a magnesium source (e.g., magnesium sulphate), and a sodium salt (e.g., sodium succinate), and (c) water. “Minimal media” may comprise elements for structural support for bacterial culture but not for serving as a nutrient source for the bacteria (e.g., agar). Preferably, “minimal media” does not contain vitamins or coenzymes. Preferably, “minimal media” does not contain amino acids or proteins.

As used herein, a “DNA” or “RNA” encoding a protein of interest refers to one or more deoxyribonucleic acids or ribonucleic acids having codons or anticodons that encode at least a part of the protein of interest. The protein of interest may be encoded by more than one deoxyribonucleic acid or ribonucleic acid, or combinations thereof. The DNA or RNA may be circular or linear, and the DNA may be single-stranded or double-stranded. In some embodiments, the DNA or RNA may be a plasmid.

As used herein, “free amino acids” refer to amino acids that are not part of a polypeptide or protein.

As used herein, “free nucleotides” refer to nucleotides that are not part of a polynucleotide and encompass corresponding nucleosides. Exemplary polynucleotides include, without limitation, plasmids, genomic DNA, and genomic RNA, which are not encompassed by “free nucleotides.” A “nucleotide” refers to a compound comprising a nucleoside moiety and a phosphate moiety. Exemplary nucleotides include, without limitation, adenosine triphosphate (ATP), uridine triphosphate (UTP), cytidine triphosphate (CTP), guanosine triphosphate (GTP), adenosine diphosphate (ADP), uridine diphosphate (UDP), cytidine diphosphate (CDP), guanosine diphosphate (GDP), adenosine monophosphate (AMP), uridine monophosphate (UMP), cytidine monophosphate (CMP), and guanosine monophosphate (GMP), deoxyadenosine triphosphate (dATP), deoxythymidine triphosphate (dTTP), deoxycytidine triphosphate (dCTP), deoxyguanosine triphosphate (dGTP), deoxyadenosine diphosphate (dADP), thymidine diphosphate (dTDP), deoxycytidine diphosphate (dCDP), deoxyguanosine diphosphate (dGDP), deoxyadenosine monophosphate (dAMP), deoxythymidine monophosphate (dTMP), deoxycytidine monophosphate (dCMP), and deoxyguanosine monophosphate (dGMP).

As used herein, “free NADH/NADPH” refers to any combination and ratio of free oxidized and reduced forms of nicotinamide adenine dinucleotide and free oxidized and reduced forms of nicotinamide adenine dinucleotide phosphate.

As used herein, “maintaining” a cell-free protein synthesis system at a certain temperature refers to keeping the cell-free protein synthesis system in a space (e.g., a room), in which the temperature thereof is that certain temperature, for an amount of time sufficient to perform cell-free protein synthesis. One or more parts of the cell-free protein synthesis system may have a temperature higher or lower than the certain temperature of the space.

Although various features of the invention may be described in the context of a single embodiment, the features may also be provided separately or in any suitable combination. Conversely, although the invention may be described herein in the context of separate embodiments for clarity, the invention may also be implemented in a single embodiment.

### Cell-Free Protein Synthesis System

The present inventors have developed a cell-free protein synthesis system that comprises a cell extract from bacteria grown in minimal media, and does not need the addition of amino acids or nucleotides. It is thought that this works via use of the aerobic respiration cycle to generate these components.

Thus, provided herein is a cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria.

In some embodiments, the pH of the cell-free protein synthesis system is from about 7.0 to about 8.0. In some embodiments, the pH of the cell-free protein synthesis system is from 7.1 to 7.9. In some embodiments, the pH of the cell-free protein synthesis system is from 7.2 to 7.8. In some embodiments, the pH of the cell-free protein synthesis system is from 7.3 to 7.7. In some embodiments, the pH of the cell-free protein synthesis system is from 7.4 to 7.6. In some embodiments, the pH of the cell-free protein synthesis system is about 7.5. In some embodiments, provided herein is a cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5.

In some embodiments, the cell-free protein synthesis system is maintained at a temperature less than about 30° C. In some embodiments, the cell-free protein synthesis system is maintained at a temperature from about 20° C. to about 30° C. In some embodiments, the cell-free protein synthesis system is maintained at a temperature from about 21° C. to about 29° C. In some embodiments, the cell-free protein synthesis system is maintained at a temperature from about 22° C. to about 28° C. In some embodiments, the cell-free protein synthesis system is maintained at a temperature from about 23° C. to about 27° C. In some embodiments, the cell-free protein synthesis system is maintained at a temperature from about 24° C. to about 36° C. In some embodiments, the cell-free protein synthesis system is maintained at a temperature that is higher than negative 20° C. In some embodiments, the cell-free protein synthesis system is maintained at a temperature about 25° C. In some embodiments, provided herein is a cell-free protein synthesis system comprising: (a) a cell-free cell extract comprising supernatant collected from lysed bacteria grown in minimal media; and (b) a reaction mixture comprising DNA or RNA encoding aa protein of interest, wherein the cell-free protein synthesis system does not comprise free amino acids, free nucleotides, or free NADH/NADPH, except when endogenous to the lysed bacteria, wherein the cell-free protein synthesis system is maintained at a temperature less than about 30° C.

In some embodiments, provided herein is a cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5, and wherein the cell-free protein synthesis system is maintained at a temperature less than about 30° C.

In some embodiments, the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer.

In some embodiments, the reaction mixture further comprises at least two of: (a) a molecular crowder; (b) a carbon source; (c) a ammonium source; (d) a potassium source; (e) a magnesium source; (f) a phosphate source; and (g) a sulfur source; and (h) a buffer.

In some embodiments, the reaction mixture further comprises at least three of: (a) a molecular crowder; (b) a carbon source; (c) a ammonium source; (d) a potassium source; (e) a magnesium source; (f) a phosphate source; and (g) a sulfur source; and (h) a buffer.

In some embodiments, the reaction mixture further comprises at least four of: (a) a molecular crowder; (b) a carbon source; (c) a ammonium source; (d) a potassium source; (e) a magnesium source; (f) a phosphate source; and (g) a sulfur source; and (h) a buffer.

In some embodiments, the reaction mixture further comprises at least five of: (a) a molecular crowder; (b) a carbon source; (c) a ammonium source; (d) a potassium source; (e) a magnesium source; (f) a phosphate source; and (g) a sulfur source; and (h) a buffer.

In some embodiments, the reaction mixture further comprises at least six of: (a) a molecular crowder; (b) a carbon source; (c) a ammonium source; (d) a potassium source; (e) a magnesium source; (f) a phosphate source; and (g) a sulfur source; and (h) a buffer.

In some embodiments, the reaction mixture further comprises at least seven of: (a) a molecular crowder; (b) a carbon source; (c) a ammonium source; (d) a potassium source; (e) a magnesium source; (f) a phosphate source; and (g) a sulfur source; and (h) a buffer.

In some embodiments, the reaction mixture further comprises: (a) a molecular crowder; (b) a carbon source; (c) a ammonium source; (d) a potassium source; (e) a magnesium source; (f) a phosphate source; and (g) a sulfur source; and optionally (h) a buffer.

In some embodiments, the reaction mixture further comprises: (a) a molecular crowder; (b) a carbon source; (c) a ammonium source; (d) a potassium source; (e) a magnesium source; (f) a phosphate source; and (g) a sulfur source; and (h) a buffer.

In some embodiments, the reaction mixture comprises NaCl. NaCl may be present at 50-250 mM, for example 100-200 mM, for example 100 mM. Such NaCl concentrations are advantageous where a cell extract from V. natriegens is to be used.

In some embodiments, provided herein is a cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer. In some embodiments, provided herein is a cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5, wherein the cell-free protein synthesis system is maintained at a temperature less than about 30° C., and wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer.

In some embodiments, the molecular crowder is chosen from PEG 400-8000, maltodextrin, Ficoll 70-400, dextran, and serum albumin, optionally bovine serum albumin. In some embodiments, the molecular crowder comprises PEG 400. In some embodiments, the molecular crowder comprises PEG 1500. In some embodiments, the molecular crowder comprises PEG 3350. In some embodiments, the molecular crowder comprises PEG 4000. In some embodiments, the molecular crowder comprises PEG 6000. In some embodiments, the molecular crowder comprises PEG 8000. In some embodiments, the molecular crowder comprises maltodextrin. In some embodiments, the molecular crowder comprises Ficoll 70. In some embodiments, the molecular crowder comprises Ficoll 400. In some embodiments, the molecular crowder comprises dextran. In some embodiments, the molecular crowder comprises serum albumin. In some embodiments, the molecular crowder comprises bovine serum albumin. In some embodiments, provided herein is a cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, and wherein the molecular crowder, if present, is chosen from PEG 400-8000, maltodextrin, Ficoll 70-400, dextran, and serum albumin, optionally bovine serum albumin.

In some embodiments, provided herein is a cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5, wherein the cell-free protein synthesis system is maintained at a temperature less than about 30° C., wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, and wherein the molecular crowder, if present, is chosen from PEG 400-8000, maltodextrin, Ficoll 70-400, dextran, and serum albumin, optionally bovine serum albumin.

In some embodiments, the concentration of the molecular crowder in the cell-free protein synthesis system is from about 0 to 40 g/L. In some embodiments, the concentration of the molecular crowder in the cell-free protein synthesis system is from 2.5 to 37.5 g/L. In some embodiments, the concentration of the molecular crowder in the cell-free protein synthesis system is from 5 to 35 g/L. In some embodiments, the concentration of the molecular crowder in the cell-free protein synthesis system is from 7.5 to 32.5 g/L. In some embodiments, the concentration of the molecular crowder in the cell-free protein synthesis system is from 10 to 30 g/L. In some embodiments, the concentration of the molecular crowder in the cell-free protein synthesis system is from 12.5 to 27.5 g/L. In some embodiments, the concentration of the molecular crowder in the cell-free protein synthesis system is from 15 to 25 g/L. In some embodiments, the concentration of the molecular crowder in the cell-free protein synthesis system is from 17.5 to 22.5 g/L. In some embodiments, the concentration of the molecular crowder in the cell-free protein synthesis system is 20 g/L. In some embodiments, provided herein is a cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, and wherein the concentration of the molecular crowder, if present, in the cell-free protein synthesis system is from about 0 to 40 g/L.

In some embodiments, provided herein is a cell-free protein synthesis system, the cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5, wherein the cell-free protein synthesis system is maintained at a temperature less than about 30° C., wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, wherein the molecular crowder, if present, is chosen from PEG 400-8000, maltodextrin, Ficoll 70-400, dextran, and serum albumin, optionally bovine serum albumin, and wherein the concentration of the molecular crowder in the cell-free protein synthesis system, if present, is from about 0 to 40 g/L.

In some embodiments, the carbon source comprises a sugar. In some embodiments, the carbon source is chosen from glucose, fructose, galactose, sucrose, lactose, gluconate, starch, maltodextrin, and maltose. In some embodiments, the carbon source comprises glucose. In some embodiments, the carbon source comprises fructose. In some embodiments, the carbon source comprises galactose. In some embodiments, the carbon source comprises sucrose. In some embodiments, the carbon source comprises lactose. In some embodiments, the carbon source comprises gluconate. In some embodiments, the carbon source comprises starch. In some embodiments, the carbon source comprises maltodextrin. In some embodiments, the carbon source comprises maltose. In some embodiments, alternatively, the carbon source does not comprise maltose. In some embodiments, provided herein is a cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, and wherein the carbon source, if present, is chosen from glucose, fructose, galactose, sucrose, lactose, gluconate, starch, maltodextrin, and maltose.

In some embodiments, provided herein is a cell-free protein synthesis system, the cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5, wherein the cell-free protein synthesis system is maintained at a temperature less than about 30° C., wherein the molecular crowder, if present, is chosen from PEG 400-8000, maltodextrin, Ficoll 70-400, dextran, and serum albumin, optionally bovine serum albumin, wherein the concentration of the molecular crowder in the cell-free protein synthesis system is from about 0 to 40 g/L, and wherein the carbon source, if present, is chosen from glucose, fructose, galactose, sucrose, lactose, gluconate, starch, maltodextrin, and maltose.

In some embodiments, the concentration of the carbon source in the cell-free protein synthesis system is from about 0 to 50 g/L. In some embodiments, the concentration of the carbon source in the cell-free protein synthesis system is from 2 to 47 g/L. In some embodiments, the concentration of the carbon source in the cell-free protein synthesis system is from 4 to 44 g/L. In some embodiments, the concentration of the carbon source in the cell-free protein synthesis system is from 6 to 41 g/L. In some embodiments, the concentration of the carbon source in the cell-free protein synthesis system is from 8 to 38 g/L. In some embodiments, the concentration of the carbon source in the cell-free protein synthesis system is from 10 to 35 g/L. In some embodiments, the concentration of the carbon source in the cell-free protein synthesis system is from 12 to 32 g/L. In some embodiments, the concentration of the carbon source in the cell-free protein synthesis system is from 14 to 29 g/L. In some embodiments, the concentration of the carbon source in the cell-free protein synthesis system is from 16 to 36 g/L. In some embodiments, the concentration of the carbon source in the cell-free protein synthesis system is from 18 to 33 g/L. In some embodiments, the concentration of the carbon source in the cell-free protein synthesis system is 20 g/L. In some embodiments, provided herein is a cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, and wherein the concentration of the carbon source in the cell-free protein synthesis system, if present, is from about 0 to 50 g/L.

In some embodiments, provided herein is a cell-free protein synthesis system, the cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5, wherein the cell-free protein synthesis system is maintained at a temperature less than about 30° C., wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, wherein the molecular crowder, if present, is chosen from PEG 400-8000, maltodextrin, Ficoll 70-400, dextran, and serum albumin, optionally bovine serum albumin, wherein the concentration of the molecular crowder, if present, in the cell-free protein synthesis system is from about 0 to 40 g/L, wherein the carbon source is chosen from glucose, fructose, galactose, sucrose, lactose, gluconate, starch, maltodextrin, and maltose, and wherein the concentration of the carbon source in the cell-free protein synthesis system is from about 0 to 50 g/L.

In some embodiments, the ammonium source comprises ammonium sulfate. In some embodiments, the concentration of the ammonium source in the cell-free protein synthesis system is from 2 to 20 mM. In some embodiments, the concentration of the ammonium source in the cell-free protein synthesis system is from 3 to 19 mM. In some embodiments, the concentration of the ammonium source in the cell-free protein synthesis system is from 4 to 18 mM. In some embodiments, the concentration of the ammonium source in the cell-free protein synthesis system is from 5 to 17 mM. In some embodiments, the concentration of the ammonium source in the cell-free protein synthesis system is from 6 to 16 mM. In some embodiments, the concentration of the ammonium source in the cell-free protein synthesis system is from 7 to 16 mM. In some embodiments, the concentration of the ammonium source in the cell-free protein synthesis system is from 8 to 16 mM. In some embodiments, the concentration of the ammonium source in the cell-free protein synthesis system is from 9 to 15 mM. In some embodiments, the concentration of the ammonium source in the cell-free protein synthesis system is from 10 to 14 mM. In some embodiments, the concentration of the ammonium source in the cell-free protein synthesis system is from 11 to 13 mM. In some embodiments, the concentration of the ammonium source in the cell-free protein synthesis system is 12 mM. In some embodiments, provided herein is a cell-free protein synthesis system, the cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, and wherein the ammonium source comprises ammonium sulfate. In some embodiments, provided herein is a cell-free protein synthesis system, the cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, and wherein the concentration of the ammonium source, if present, in the cell-free protein synthesis system is from 2 to 20 mM.

In some embodiments, provided herein is a cell-free protein synthesis system, the cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5, wherein the cell-free protein synthesis system is maintained at a temperature less than about 30° C., wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, wherein the molecular crowder, if present, is chosen from PEG 400-8000, maltodextrin, Ficoll 70-400, dextran, and serum albumin, optionally bovine serum albumin, wherein the concentration of the molecular crowder, if present, in the cell-free protein synthesis system is from about 0 to 40 g/L, wherein the carbon source, if present, is chosen from glucose, fructose, galactose, sucrose, lactose, gluconate, starch, maltodextrin, and maltose, wherein the concentration of the carbon source, if present, in the cell-free protein synthesis system is from about 0 to 50 g/L, wherein the ammonium source, if present, comprises ammonium sulfate, and wherein the concentration of the ammonium source in the cell-free protein synthesis system is from 2 to 20 mM.

In some embodiments, the potassium source is chosen from potassium glutamate and potassium gluconate. In some embodiments, the potassium source comprises potassium glutamate and potassium gluconate. In some embodiments, the potassium source comprises potassium glutamate. In some embodiments, the potassium source comprises potassium gluconate. In some embodiments, provided herein is a cell-free protein synthesis system, the cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, and wherein the potassium source, if present, is chosen from potassium glutamate and potassium gluconate.

In some embodiments, provided herein is a cell-free protein synthesis system, the cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5, wherein the cell-free protein synthesis system is maintained at a temperature less than about 30° C., wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, wherein the molecular crowder, if present, is chosen from PEG 400-8000, maltodextrin, Ficoll 70-400, dextran, and serum albumin, optionally bovine serum albumin, wherein the concentration of the molecular crowder, if present, in the cell-free protein synthesis system is from about 0 to 40 g/L, wherein the carbon source, if present, is chosen from glucose, fructose, galactose, sucrose, lactose, gluconate, starch, maltodextrin, and maltose, wherein the concentration of the carbon source, if present, in the cell-free protein synthesis system is from about 0 to 50 g/L, wherein the ammonium source, if present, comprises ammonium sulfate, wherein the concentration of the ammonium source, if present, in the cell-free protein synthesis system is from 2 to 20 mM, and wherein the potassium source, if present, is chosen from potassium glutamate and potassium gluconate.

In some embodiments, the concentration of the potassium glutamate in the cell-free protein synthesis system is from about 10 to 270 mM. In some embodiments, the concentration of the potassium glutamate in the cell-free protein synthesis system is from 11 to 240 mM. In some embodiments, the concentration of the potassium glutamate in the cell-free protein synthesis system is from 12 to 210 mM. In some embodiments, the concentration of the potassium glutamate in the cell-free protein synthesis system is from 13 to 180 mM. In some embodiments, the concentration of the potassium glutamate in the cell-free protein synthesis system is from 14 to 150 mM. In some embodiments, the concentration of the potassium glutamate in the cell-free protein synthesis system is from 15 to 120 mM. In some embodiments, the concentration of the potassium glutamate in the cell-free protein synthesis system is from 16 to 90 mM. In some embodiments, the concentration of the potassium glutamate in the cell-free protein synthesis system is from 17 to 60 mM. In some embodiments, the concentration of the potassium glutamate in the cell-free protein synthesis system is from 18 to 30 mM. In some embodiments, the concentration of the potassium glutamate in the cell-free protein synthesis system is from 18 to 28 mM. In some embodiments, the concentration of the potassium glutamate in the cell-free protein synthesis system is from 18 to 26 mM. In some embodiments, the concentration of the potassium glutamate in the cell-free protein synthesis system is from 18 to 24 mM. In some embodiments, the concentration of the potassium glutamate in the cell-free protein synthesis system is from 19 to 24 mM. In some embodiments, the concentration of the potassium glutamate in the cell-free protein synthesis system is from 19 to 22 mM. In some embodiments, the concentration of the potassium glutamate in the cell-free protein synthesis system is from 19 to 21 mM. In some embodiments, the concentration of the potassium glutamate in the cell-free protein synthesis system is 20 mM. In some embodiments, provided herein is a cell-free protein synthesis system, the cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, wherein the potassium source, if present, is chosen from potassium glutamate and potassium gluconate, and wherein the concentration of the potassium glutamate, if present, in the cell-free protein synthesis system is from about 10 to 270 mM.

In some embodiments, provided herein is a cell-free protein synthesis system, the cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5, wherein the cell-free protein synthesis system is maintained at a temperature less than about 30° C., wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, wherein the molecular crowder, if present, is chosen from PEG 400-8000, maltodextrin, Ficoll 70-400, dextran, and serum albumin, optionally bovine serum albumin, wherein the concentration of the molecular crowder, if present, in the cell-free protein synthesis system is from about 0 to 40 g/L, wherein the carbon source, if present, is chosen from glucose, fructose, galactose, sucrose, lactose, gluconate, starch, maltodextrin, and maltose, wherein the concentration of the carbon source, if present, in the cell-free protein synthesis system is from about 0 to 50 g/L, wherein the ammonium source, if present, comprises ammonium sulfate, wherein the concentration of the ammonium source, if present, in the cell-free protein synthesis system is from 2 to 20 mM, wherein the potassium source, if present, is chosen from potassium glutamate and potassium gluconate, and wherein the concentration of the potassium glutamate, if present, in the cell-free protein synthesis system is from about 10 to 270 mM.

In some embodiments, the concentration of the potassium gluconate in the cell-free protein synthesis system is from about 10 to 250 mM. In some embodiments, the concentration of the potassium gluconate in the cell-free protein synthesis system is from 15 to 240 mM. In some embodiments, the concentration of the potassium gluconate in the cell-free protein synthesis system is from 20 to 230 mM. In some embodiments, the concentration of the potassium gluconate in the cell-free protein synthesis system is from 25 to 220 mM. In some embodiments, the concentration of the potassium gluconate in the cell-free protein synthesis system is from 30 to 210 mM. In some embodiments, the concentration of the potassium gluconate in the cell-free protein synthesis system is from 35 to 200 mM. In some embodiments, the concentration of the potassium gluconate in the cell-free protein synthesis system is from 40 to 190 mM. In some embodiments, the concentration of the potassium gluconate in the cell-free protein synthesis system is from 45 to 180 mM. In some embodiments, the concentration of the potassium gluconate in the cell-free protein synthesis system is from 50 to 170 mM. In some embodiments, the concentration of the potassium gluconate in the cell-free protein synthesis system is from 55 to 160 mM. In some embodiments, the concentration of the potassium gluconate in the cell-free protein synthesis system is from 60 to 150 mM. In some embodiments, the concentration of the potassium gluconate in the cell-free protein synthesis system is from 70 to 140 mM. In some embodiments, the concentration of the potassium gluconate in the cell-free protein synthesis system is from 80 to 130 mM. In some embodiments, the concentration of the potassium gluconate in the cell-free protein synthesis system is from 90 to 120 mM. In some embodiments, the concentration of the potassium gluconate in the cell-free protein synthesis system is from 95 to 110 mM. In some embodiments, the concentration of the potassium gluconate in the cell-free protein synthesis system is from 95 to 105 mM. In some embodiments, the concentration of the potassium gluconate in the cell-free protein synthesis system is 100 mM. In some embodiments, provided herein is a cell-free protein synthesis system, the cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, wherein the potassium source, if present, is chosen from potassium glutamate and potassium gluconate, and wherein the concentration of the potassium gluconate, if present, in the cell-free protein synthesis system is from about 10 to 250 mM.

In some embodiments, provided herein is a cell-free protein synthesis system, the cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5, wherein the cell-free protein synthesis system is maintained at a temperature less than about 30° C., wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, wherein the molecular crowder, if present, is chosen from PEG 400-8000, maltodextrin, Ficoll 70-400, dextran, and serum albumin, optionally bovine serum albumin, wherein the concentration of the molecular crowder, if present, in the cell-free protein synthesis system is from about 0 to 40 g/L, wherein the carbon source, if present, is chosen from glucose, fructose, galactose, sucrose, lactose, gluconate, starch, maltodextrin, and maltose, wherein the concentration of the carbon source, if present, in the cell-free protein synthesis system is from about 0 to 50 g/L, wherein the ammonium source, if present, comprises ammonium sulfate, wherein the concentration of the ammonium source, if present, in the cell-free protein synthesis system is from 2 to 20 mM, wherein the potassium source, if present, is chosen from potassium glutamate and potassium gluconate, wherein the concentration of the potassium glutamate, if present, in the cell-free protein synthesis system is from about 10 to 270 mM, and wherein the concentration of the potassium gluconate, if present, in the cell-free protein synthesis system is from about 10 to 250 mM.

In some embodiments, the magnesium source comprises magnesium glutamate. In some embodiments, the concentration of the magnesium source in the cell-free protein synthesis system is from 0 to about 10 mM. In some embodiments, the concentration of the magnesium source in the cell-free protein synthesis system is from 0.5 to 9.25 mM. In some embodiments, the concentration of the magnesium source in the cell-free protein synthesis system is from 1 to 8.5 mM. In some embodiments, the concentration of the magnesium source in the cell-free protein synthesis system is from 1.5 to 7.75 mM. In some embodiments, the concentration of the magnesium source in the cell-free protein synthesis system is from 2 to 7 mM. In some embodiments, the concentration of the magnesium source in the cell-free protein synthesis system is from 2.5 to 6.25 mM. In some embodiments, the concentration of the magnesium source in the cell-free protein synthesis system is from 3 to 5.5 mM. In some embodiments, the concentration of the magnesium source in the cell-free protein synthesis system is from 3.5 to 4.75 mM. In some embodiments, the concentration of the magnesium source in the cell-free protein synthesis system is from 3.5 to 4.5 mM. In some embodiments, the concentration of the magnesium source in the cell-free protein synthesis system is from 3.75 to 4.25 mM. In some embodiments, the concentration of the magnesium source in the cell-free protein synthesis system is 4 mM. In some embodiments, provided herein is a cell-free protein synthesis system, the cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, and wherein the magnesium source, if present, comprises magnesium glutamate. In some embodiments, provided herein is a cell-free protein synthesis system, the cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, wherein the magnesium source, if present, comprises magnesium glutamate, and wherein the concentration of the magnesium source, if present, in the cell-free protein synthesis system is from 0 to about 10 mM.

In some embodiments, provided herein is a cell-free protein synthesis system, the cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5, wherein the cell-free protein synthesis system is maintained at a temperature less than about 30° C., wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, wherein the molecular crowder, if present, is chosen from PEG 400-8000, maltodextrin, Ficoll 70-400, dextran, and serum albumin, optionally bovine serum albumin, wherein the concentration of the molecular crowder, if present, in the cell-free protein synthesis system is from about 0 to 40 g/L, wherein the carbon source, if present, is chosen from glucose, fructose, galactose, sucrose, lactose, gluconate, starch, maltodextrin, and maltose, wherein the concentration of the carbon source, if present, in the cell-free protein synthesis system is from about 0 to 50 g/L, wherein the ammonium source, if present, comprises ammonium sulfate, wherein the concentration of the ammonium source, if present, in the cell-free protein synthesis system is from 2 to 20 mM, wherein the potassium source, if present, is chosen from potassium glutamate and potassium gluconate, wherein the concentration of the potassium glutamate, if present, in the cell-free protein synthesis system is from about 10 to 270 mM, wherein the concentration of the potassium gluconate in the cell-free protein synthesis system, if present, is from about 10 to 250 mM, wherein the magnesium source, if present, comprises magnesium glutamate, and wherein the concentration of the magnesium source, if present, in the cell-free protein synthesis system is from 0 to about 10 mM.

In some embodiments, the phosphate source comprises potassium phosphate. In some embodiments, the concentration of the phosphate source in the cell-free protein synthesis system is from about 5 to about 23.5 mM. In some embodiments, the concentration of the phosphate source in the cell-free protein synthesis system is from 6 to 22 mM. In some embodiments, the concentration of the phosphate source in the cell-free protein synthesis system is from 7 to 20.5 mM. In some embodiments, the concentration of the phosphate source in the cell-free protein synthesis system is from 8 to 19 mM. In some embodiments, the concentration of the phosphate source in the cell-free protein synthesis system is from 9 to 17.5 mM. In some embodiments, the concentration of the phosphate source in the cell-free protein synthesis system is from 10 to 16 mM. In some embodiments, the concentration of the phosphate source in the cell-free protein synthesis system is from 11 to 14.5 mM. In some embodiments, the concentration of the phosphate source in the cell-free protein synthesis system is from 12 to 14 mM. In some embodiments, the concentration of the phosphate source in the cell-free protein synthesis system is from 12.5 to 13.5 mM. In some embodiments, the concentration of the phosphate source in the cell-free protein synthesis system is 13 mM. In some embodiments, provided herein is a cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, and wherein the phosphate source comprises potassium phosphate. In some embodiments, provided herein is a cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, and wherein the concentration of the phosphate source in the cell-free protein synthesis system is from about 5 to about 23.5 mM.

In some embodiments, provided herein is a cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5, wherein the cell-free protein synthesis system is maintained at a temperature less than about 30° C., wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, wherein the molecular crowder is chosen from PEG 400-8000, maltodextrin, Ficoll 70-400, dextran, and serum albumin, optionally bovine serum albumin, wherein the concentration of the molecular crowder, if present, in the cell-free protein synthesis system is from about 0 to 40 g/L, wherein the carbon source, if present, is chosen from glucose, fructose, galactose, sucrose, lactose, gluconate, starch, maltodextrin, and maltose, wherein the concentration of the carbon source, if present, in the cell-free protein synthesis system is from about 0 to 50 g/L, wherein the ammonium source, if present, comprises ammonium sulfate, wherein the concentration of the ammonium source, if present, in the cell-free protein synthesis system is from 2 to 20 mM, wherein the potassium source, if present, is chosen from potassium glutamate and potassium gluconate, wherein the concentration of the potassium glutamate, if present, in the cell-free protein synthesis system is from about 10 to 270 mM, wherein the concentration of the potassium gluconate, if present, in the cell-free protein synthesis system is from about 10 to 250 mM, wherein the magnesium source, if present, comprises magnesium glutamate, wherein the concentration of the magnesium source, if present, in the cell-free protein synthesis system is from 0 to about 10 mM, wherein the phosphate source, if present, comprises potassium phosphate, and wherein the concentration of the phosphate source, if present, in the cell-free protein synthesis system is from about 5 to about 23.5 mM.

In some embodiments, the sulfur source comprises ammonium sulfate. In some embodiments, the concentration of the sulfur source in the cell-free protein synthesis system is from 2 to 20 mM. In some embodiments, the concentration of the sulfur source in the cell-free protein synthesis system is from 3 to 19 mM. In some embodiments, the concentration of the sulfur source in the cell-free protein synthesis system is from 4 to 18 mM. In some embodiments, the concentration of the sulfur source in the cell-free protein synthesis system is from 5 to 17 mM. In some embodiments, the concentration of the sulfur source in the cell-free protein synthesis system is from 6 to 16 mM. In some embodiments, the concentration of the sulfur source in the cell-free protein synthesis system is from 7 to 15 mM. In some embodiments, the concentration of the sulfur source in the cell-free protein synthesis system is from 8 to 15 mM. In some embodiments, the concentration of the sulfur source in the cell-free protein synthesis system is from 9 to 14 mM. In some embodiments, the concentration of the sulfur source in the cell-free protein synthesis system is from 10 to 14 mM. In some embodiments, the concentration of the sulfur source in the cell-free protein synthesis system is from 11 to 13 mM. In some embodiments, the concentration of the sulfur source in the cell-free protein synthesis system is from 11.5 to 12.5 mM. In some embodiments, the concentration of the sulfur source in the cell-free protein synthesis system is 12 mM. In some embodiments, provided herein is a cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, and wherein the sulfur source comprises ammonium sulfate. In some embodiments, provided herein is a cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, and wherein the concentration of the sulfur source, if present, in the cell-free protein synthesis system is from 2 to 20 mM.

In some embodiments, provided herein is a cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5, wherein the cell-free protein synthesis system is maintained at a temperature less than about 30° C., wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, wherein the molecular crowder, if present, is chosen from PEG 400-8000, maltodextrin, Ficoll 70-400, dextran, and serum albumin, optionally bovine serum albumin, wherein the concentration of the molecular crowder, if present, in the cell-free protein synthesis system is from about 0 to 40 g/L, wherein the carbon source, if present, is chosen from glucose, fructose, galactose, sucrose, lactose, gluconate, starch, maltodextrin, and maltose, wherein the concentration of the carbon source, if present, in the cell-free protein synthesis system is from about 0 to 50 g/L, wherein the ammonium source, if present, comprises ammonium sulfate, wherein the concentration of the ammonium source, if present, in the cell-free protein synthesis system is from 2 to 20 mM, wherein the potassium source, if present, is chosen from potassium glutamate and potassium gluconate, wherein the concentration of the potassium glutamate, if present, in the cell-free protein synthesis system is from about 10 to 270 mM, wherein the concentration of the potassium gluconate, if present, in the cell-free protein synthesis system is from about 10 to 250 mM, wherein the magnesium source, if present, comprises magnesium glutamate, wherein the concentration of the magnesium source, if present, in the cell-free protein synthesis system is from 0 to about 10 mM, wherein the phosphate source, if present, comprises potassium phosphate, wherein the concentration of the phosphate source, if present, in the cell-free protein synthesis system is from about 5 to about 23.5 mM, wherein the sulfur source, if present, comprises ammonium sulfate, and wherein the concentration of the sulfur source, if present, in the cell-free protein synthesis system is from 2 to 20 mM.

In some embodiments, the cell-free protein synthesis system comprises the buffer. In some embodiments, the buffer comprises 3-(N-morpholino)propanesulfonic acid (MOPS) buffer. In some embodiments, the concentration of the buffer in the cell-free protein synthesis system is from 50 to 300 mM. In some embodiments, the concentration of the buffer in the cell-free protein synthesis system is from 55 to 280 mM. In some embodiments, the concentration of the buffer in the cell-free protein synthesis system is from 60 to 260 mM. In some embodiments, the concentration of the buffer in the cell-free protein synthesis system is from 65 to 240 mM. In some embodiments, the concentration of the buffer in the cell-free protein synthesis system is from 70 to 220 mM. In some embodiments, the concentration of the buffer in the cell-free protein synthesis system is from 75 to 200 mM. In some embodiments, the concentration of the buffer in the cell-free protein synthesis system is from 80 to 180 mM. In some embodiments, the concentration of the buffer in the cell-free protein synthesis system is from 85 to 160 mM. In some embodiments, the concentration of the buffer in the cell-free protein synthesis system is from 90 to 140 mM. In some embodiments, the concentration of the buffer in the cell-free protein synthesis system is from 95 to 120 mM. In some embodiments, the concentration of the buffer in the cell-free protein synthesis system is from 95 to 110 mM. In some embodiments, the concentration of the buffer in the cell-free protein synthesis system is from 95 to 105 mM. In some embodiments, the concentration of the buffer in the cell-free protein synthesis system is 100 mM. In some embodiments, provided herein is a cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, and wherein the buffer, if present, comprises 3-(N-morpholino)propanesulfonic acid (MOPS) buffer. In some embodiments, provided herein is a cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, and wherein the concentration of the buffer, if present, in the cell-free protein synthesis system is from 50 to 300 mM.

In some embodiments, provided herein is a cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5, wherein the cell-free protein synthesis system is maintained at a temperature less than about 30° C., wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, wherein the molecular crowder, if present, is chosen from PEG 400-8000, maltodextrin, Ficoll 70-400, dextran, and serum albumin, optionally bovine serum albumin, wherein the concentration of the molecular crowder, if present, in the cell-free protein synthesis system is from about 0 to 40 g/L, wherein the carbon source, if present, is chosen from glucose, fructose, galactose, sucrose, lactose, gluconate, starch, maltodextrin, and maltose, wherein the concentration of the carbon source, if present, in the cell-free protein synthesis system is from about 0 to 50 g/L, wherein the ammonium source, if present, comprises ammonium sulfate, wherein the concentration of the ammonium source, if present, in the cell-free protein synthesis system is from 2 to 20 mM, wherein the potassium source, if present, is chosen from potassium glutamate and potassium gluconate, wherein the concentration of the potassium glutamate, if present, in the cell-free protein synthesis system is from about 10 to 270 mM, wherein the concentration of the potassium gluconate, if present, in the cell-free protein synthesis system is from about 10 to 250 mM, wherein the magnesium source, if present, comprises magnesium glutamate, wherein the concentration of the magnesium source in the cell-free protein synthesis system is from 0 to about 10 mM, wherein the phosphate source, if present, comprises potassium phosphate, wherein the concentration of the phosphate source, if present, in the cell-free protein synthesis system is from about 5 to about 23.5 mM, wherein the sulfur source, if present, comprises ammonium sulfate, wherein the concentration of the sulfur source, if present, in the cell-free protein synthesis system is from 2 to 20 mM, wherein the buffer, if present, comprises 3-(N-morpholino)propanesulfonic acid (MOPS) buffer, and wherein the concentration of the buffer in the cell-free protein synthesis system is from 50 to 300 mM.

In some embodiments, the concentration of the cell-free cell extract in the cell-free protein synthesis system is from 10% to 70% (v/v). In some embodiments, the concentration of the cell-free cell extract in the cell-free protein synthesis system is from 12% to 66% (v/v). In some embodiments, the concentration of the cell-free cell extract in the cell-free protein synthesis system is from 14% to 62% (v/v). In some embodiments, the concentration of the cell-free cell extract in the cell-free protein synthesis system is from 16% to 58% (v/v). In some embodiments, the concentration of the cell-free cell extract in the cell-free protein synthesis system is from 18% to 54% (v/v). In some embodiments, the concentration of the cell-free cell extract in the cell-free protein synthesis system is from 20% to 50% (v/v). In some embodiments, the concentration of the cell-free cell extract in the cell-free protein synthesis system is from 22% to 46% (v/v). In some embodiments, the concentration of the cell-free cell extract in the cell-free protein synthesis system is from 24% to 42% (v/v). In some embodiments, the concentration of the cell-free cell extract in the cell-free protein synthesis system is from 26% to 38% (v/v). In some embodiments, the concentration of the cell-free cell extract in the cell-free protein synthesis system is from 28% to 34% (v/v). In some embodiments, the concentration of the cell-free cell extract in the cell-free protein synthesis system is from 29% to 32% (v/v). In some embodiments, the concentration of the cell-free cell extract in the cell-free protein synthesis system is from 29% to 31% (v/v). In some embodiments, the concentration of the cell-free cell extract in the cell-free protein synthesis system is 30% (v/v). In some embodiments, provided herein is a cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the concentration of the cell-free cell extract in the cell-free protein synthesis system is from 10% to 70% (v/v).

In some embodiments, provided herein is a cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5, wherein the cell-free protein synthesis system is maintained at a temperature less than about 30° C., wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, wherein the molecular crowder is chosen from PEG 400-8000, maltodextrin, Ficoll 70-400, dextran, and serum albumin, optionally bovine serum albumin, wherein the concentration of the molecular crowder, if present, in the cell-free protein synthesis system is from about 0 to 40 g/L, wherein the carbon source, if present, is chosen from glucose, fructose, galactose, sucrose, lactose, gluconate, starch, maltodextrin, and maltose, wherein the concentration of the carbon source in the cell-free protein synthesis system, if present, is from about 0 to 50 g/L, wherein the ammonium source, if present, comprises ammonium sulfate, wherein the concentration of the ammonium source, if present, in the cell-free protein synthesis system is from 2 to 20 mM, wherein the potassium source, if present, is chosen from potassium glutamate and potassium gluconate, wherein the concentration of the potassium glutamate in the cell-free protein synthesis system, if present, is from about 10 to 270 mM, wherein the concentration of the potassium gluconate, if present, in the cell-free protein synthesis system is from about 10 to 250 mM, wherein the magnesium source, if present, comprises magnesium glutamate, wherein the concentration of the magnesium source, if present, in the cell-free protein synthesis system is from 0 to about 10 mM, wherein the phosphate source, if present, comprises potassium phosphate, wherein the concentration of the phosphate source in the cell-free protein synthesis system is from about 5 to about 23.5 mM, wherein the sulfur source, if present, comprises ammonium sulfate, wherein the concentration of the sulfur source, if present, in the cell-free protein synthesis system is from 2 to 20 mM, wherein the buffer, if present, comprises 3-(N-morpholino)propanesulfonic acid (MOPS) buffer, wherein the concentration of the buffer in the cell-free protein synthesis system is from 50 to 300 mM, and wherein the concentration of the cell-free cell extract in the cell-free protein synthesis system is from 10% to 70% (v/v).

In some embodiments, the concentration of the DNA or RNA encoding the protein of interest in the cell-free protein synthesis system is from 1 to 100 μg/mL. In some embodiments, the concentration of the DNA or RNA encoding the protein of interest in the cell-free protein synthesis system is from 2 to 90 μg/mL. In some embodiments, the concentration of the DNA or RNA encoding the protein of interest in the cell-free protein synthesis system is from 3 to 80 g/mL. In some embodiments, the concentration of the DNA or RNA encoding the protein of interest in the cell-free protein synthesis system is from 4 to 70 μg/mL. In some embodiments, the concentration of the DNA or RNA encoding the protein of interest in the cell-free protein synthesis system is from 5 to 60 μg/mL. In some embodiments, the concentration of the DNA or RNA encoding the protein of interest in the cell-free protein synthesis system is from 6 to 50 μg/mL. In some embodiments, the concentration of the DNA or RNA encoding the protein of interest in the cell-free protein synthesis system is from 7 to 40 μg/mL. In some embodiments, the concentration of the DNA or RNA encoding the protein of interest in the cell-free protein synthesis system is from 8 to 30 μg/mL. In some embodiments, the concentration of the DNA or RNA encoding the protein of interest in the cell-free protein synthesis system is from 9 to 20 μg/mL. In some embodiments, the concentration of the DNA or RNA encoding the protein of interest in the cell-free protein synthesis system is from 9 to 15 μg/mL. In some embodiments, the concentration of the DNA or RNA encoding the protein of interest in the cell-free protein synthesis system is from 9.5 to 12.5 g/mL. In some embodiments, the concentration of the DNA or RNA encoding the protein of interest in the cell-free protein synthesis system is from 9.5 to 11.5 μg/mL. In some embodiments, the concentration of the DNA or RNA encoding the protein of interest in the cell-free protein synthesis system is from 9.5 to 11 μg/mL. In some embodiments, the concentration of the DNA or RNA encoding the protein of interest in the cell-free protein synthesis system is 10 μg/mL. In some embodiments, provided herein is a cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, and wherein the concentration of the DNA or RNA encoding the protein of interest in the cell-free protein synthesis system is from 1 to 100 μg/mL.

In some embodiments, provided herein is a cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5, wherein the cell-free protein synthesis system is maintained at a temperature less than about 30° C., wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, wherein the molecular crowder is chosen from PEG 400-8000, maltodextrin, Ficoll 70-400, dextran, and serum albumin, optionally bovine serum albumin, wherein the concentration of the molecular crowder, if present, in the cell-free protein synthesis system is from about 0 to 40 g/L, wherein the carbon source, if present, is chosen from glucose, fructose, galactose, sucrose, lactose, gluconate, starch, maltodextrin, and maltose, wherein the concentration of the carbon source in the cell-free protein synthesis system is from about 0 to 50 g/L, wherein the ammonium source, if present, comprises ammonium sulfate, wherein the concentration of the ammonium source in the cell-free protein synthesis system is from 2 to 20 mM, wherein the potassium source, if present, is chosen from potassium glutamate and potassium gluconate, wherein the concentration of the potassium glutamate, if present, in the cell-free protein synthesis system is from about 10 to 270 mM, wherein the concentration of the potassium gluconate in the cell-free protein synthesis system is from about 10 to 250 mM, wherein the magnesium source, if present, comprises magnesium glutamate, wherein the concentration of the magnesium source, if present, in the cell-free protein synthesis system is from 0 to about 10 mM, wherein the phosphate source, if present, comprises potassium phosphate, wherein the concentration of the phosphate source, if present, in the cell-free protein synthesis system is from about 5 to about 23.5 mM, wherein the sulfur source, if present, comprises ammonium sulfate, wherein the concentration of the sulfur source in the cell-free protein synthesis system is from 2 to 20 mM, wherein the buffer, if present, comprises 3-(N-morpholino)propanesulfonic acid (MOPS) buffer, wherein the concentration of the buffer in the cell-free protein synthesis system is from 50 to 300 mM, wherein the concentration of the cell-free cell extract in the cell-free protein synthesis system is from 10% to 70% (v/v), and wherein the concentration of the DNA or RNA encoding the protein of interest in the cell-free protein synthesis system is from 1 to 100 μg/mL.

In some embodiments, the cell-free protein synthesis system comprises free amino acids endogenous to the lysed bacteria, wherein the concentration of free amino acids in the cell-free protein synthesis system is less than 2 mM. In some embodiments, the cell-free protein synthesis system comprises free amino acids endogenous to the lysed bacteria, wherein the concentration of free amino acids in the cell-free protein synthesis system is less than 1.9 mM. In some embodiments, the cell-free protein synthesis system comprises free amino acids endogenous to the lysed bacteria, wherein the concentration of free amino acids in the cell-free protein synthesis system is less than 1.8 mM. In some embodiments, the cell-free protein synthesis system comprises free amino acids endogenous to the lysed bacteria, wherein the concentration of free amino acids in the cell-free protein synthesis system is less than 1.7 mM. In some embodiments, the cell-free protein synthesis system comprises free amino acids endogenous to the lysed bacteria, wherein the concentration of free amino acids in the cell-free protein synthesis system is less than 1.6 mM. In some embodiments, the cell-free protein synthesis system comprises free amino acids endogenous to the lysed bacteria, wherein the concentration of free amino acids in the cell-free protein synthesis system is less than 1.5 mM. In some embodiments, the cell-free protein synthesis system comprises free amino acids endogenous to the lysed bacteria, wherein the concentration of free amino acids in the cell-free protein synthesis system is less than 1.4 mM. In some embodiments, the cell-free protein synthesis system comprises free amino acids endogenous to the lysed bacteria, wherein the concentration of free amino acids in the cell-free protein synthesis system is less than 1.3 mM. In some embodiments, the cell-free protein synthesis system comprises free amino acids endogenous to the lysed bacteria, wherein the concentration of free amino acids in the cell-free protein synthesis system is less than 1.2 mM. In some embodiments, the cell-free protein synthesis system comprises free amino acids endogenous to the lysed bacteria, wherein the concentration of free amino acids in the cell-free protein synthesis system is less than 1.1 mM. In some embodiments, the cell-free protein synthesis system comprises free amino acids endogenous to the lysed bacteria, wherein the concentration of free amino acids in the cell-free protein synthesis system is less than 1 mM. In some embodiments, the cell-free protein synthesis system comprises free amino acids endogenous to the lysed bacteria, wherein the concentration of free amino acids in the cell-free protein synthesis system is less than 0.9 mM. In some embodiments, the cell-free protein synthesis system comprises free amino acids endogenous to the lysed bacteria, wherein the concentration of free amino acids in the cell-free protein synthesis system is less than 0.8 mM. In some embodiments, the cell-free protein synthesis system comprises free amino acids endogenous to the lysed bacteria, wherein the concentration of free amino acids in the cell-free protein synthesis system is less than 0.7 mM. In some embodiments, the cell-free protein synthesis system comprises free amino acids endogenous to the lysed bacteria, wherein the concentration of free amino acids in the cell-free protein synthesis system is less than 0.6 mM. In some embodiments, the cell-free protein synthesis system comprises free amino acids endogenous to the lysed bacteria, wherein the concentration of free amino acids in the cell-free protein synthesis system is less than 0.5 mM. In some embodiments, the cell-free protein synthesis system comprises free amino acids endogenous to the lysed bacteria, wherein the concentration of free amino acids in the cell-free protein synthesis system is less than 0.4 mM. In some embodiments, the cell-free protein synthesis system comprises free amino acids endogenous to the lysed bacteria, wherein the concentration of free amino acids in the cell-free protein synthesis system is less than 0.3 mM. In some embodiments, the cell-free protein synthesis system comprises free amino acids endogenous to the lysed bacteria, wherein the concentration of free amino acids in the cell-free protein synthesis system is less than 0.2 mM. In some embodiments, the cell-free protein synthesis system comprises free amino acids endogenous to the lysed bacteria, wherein the concentration of free amino acids in the cell-free protein synthesis system is less than 0.1 mM. In some embodiments, the cell-free protein synthesis system comprises free amino acids endogenous to the lysed bacteria, wherein the concentration of free amino acids in the cell-free protein synthesis system is less than 0.05 mM. In some embodiments, the cell-free protein synthesis system comprises free amino acids endogenous to the lysed bacteria, wherein the concentration of free amino acids in the cell-free protein synthesis system is less than 0.025 mM. In some embodiments, the cell-free protein synthesis system comprises free amino acids endogenous to the lysed bacteria, wherein the concentration of free amino acids in the cell-free protein synthesis system is less than 0.01 mM. In some embodiments, provided herein is a cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the cell-free protein synthesis system comprises free amino acids endogenous to the lysed bacteria, and wherein the concentration of free amino acids in the cell-free protein synthesis system is less than 2 mM.

In some embodiments, provided herein is a cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5, wherein the cell-free protein synthesis system is maintained at a temperature less than about 30° C., wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, wherein the molecular crowder is chosen from PEG 400-8000, maltodextrin, Ficoll 70-400, dextran, and serum albumin, optionally bovine serum albumin, wherein the concentration of the molecular crowder, if present, in the cell-free protein synthesis system is from about 0 to 40 g/L, wherein the carbon source, if present, is chosen from glucose, fructose, galactose, sucrose, lactose, gluconate, starch, maltodextrin, and maltose, wherein the concentration of the carbon source, if present, in the cell-free protein synthesis system is from about 0 to 50 g/L, wherein the ammonium source, if present, comprises ammonium sulfate, wherein the concentration of the ammonium source, if present, in the cell-free protein synthesis system is from 2 to 20 mM, wherein the potassium source is chosen from potassium glutamate and potassium gluconate, wherein the concentration of the potassium glutamate, if present, in the cell-free protein synthesis system is from about 10 to 270 mM, wherein the concentration of the potassium gluconate in the cell-free protein synthesis system is from about 10 to 250 mM, wherein the magnesium source, if present, comprises magnesium glutamate, wherein the concentration of the magnesium source in the cell-free protein synthesis system is from 0 to about 10 mM, wherein the phosphate source, if present, comprises potassium phosphate, wherein the concentration of the phosphate source, if present, in the cell-free protein synthesis system is from about 5 to about 23.5 mM, wherein the sulfur source, if present, comprises ammonium sulfate, wherein the concentration of the sulfur source, if present, in the cell-free protein synthesis system is from 2 to 20 mM, wherein the buffer, if present, comprises 3-(N-morpholino)propanesulfonic acid (MOPS) buffer, wherein the concentration of the buffer in the cell-free protein synthesis system is from 50 to 300 mM, wherein the concentration of the cell-free cell extract in the cell-free protein synthesis system is from 10% to 70% (v/v), wherein the concentration of the DNA or RNA encoding the protein of interest in the cell-free protein synthesis system is from 1 to 100 μg/mL, wherein the cell-free protein synthesis system comprises free amino acids endogenous to the lysed bacteria, and wherein the concentration of free amino acids in the cell-free protein synthesis system is less than 2 mM.

In some embodiments, the cell-free protein synthesis system comprises free nucleotides endogenous to the lysed bacteria, wherein the concentration of free nucleotides in the cell-free protein synthesis system is less than 1.5 mM. In some embodiments, the cell-free protein synthesis system comprises free nucleotides endogenous to the lysed bacteria, wherein the concentration of free nucleotides in the cell-free protein synthesis system is less than 1.4 mM. In some embodiments, the cell-free protein synthesis system comprises free nucleotides endogenous to the lysed bacteria, wherein the concentration of free nucleotides in the cell-free protein synthesis system is less than 1.3 mM. In some embodiments, the cell-free protein synthesis system comprises free nucleotides endogenous to the lysed bacteria, wherein the concentration of free nucleotides in the cell-free protein synthesis system is less than 1.2 mM. In some embodiments, the cell-free protein synthesis system comprises free nucleotides endogenous to the lysed bacteria, wherein the concentration of free nucleotides in the cell-free protein synthesis system is less than 1.1 mM. In some embodiments, the cell-free protein synthesis system comprises free nucleotides endogenous to the lysed bacteria, wherein the concentration of free nucleotides in the cell-free protein synthesis system is less than 1 mM. In some embodiments, the cell-free protein synthesis system comprises free nucleotides endogenous to the lysed bacteria, wherein the concentration of free nucleotides in the cell-free protein synthesis system is less than 0.9 mM. In some embodiments, the cell-free protein synthesis system comprises free nucleotides endogenous to the lysed bacteria, wherein the concentration of free nucleotides in the cell-free protein synthesis system is less than 0.8 mM. In some embodiments, the cell-free protein synthesis system comprises free nucleotides endogenous to the lysed bacteria, wherein the concentration of free nucleotides in the cell-free protein synthesis system is less than 0.7 mM. In some embodiments, the cell-free protein synthesis system comprises free nucleotides endogenous to the lysed bacteria, wherein the concentration of free nucleotides in the cell-free protein synthesis system is less than 0.6 mM. In some embodiments, the cell-free protein synthesis system comprises free nucleotides endogenous to the lysed bacteria, wherein the concentration of free nucleotides in the cell-free protein synthesis system is less than 0.5 mM. In some embodiments, the cell-free protein synthesis system comprises free nucleotides endogenous to the lysed bacteria, wherein the concentration of free nucleotides in the cell-free protein synthesis system is less than 0.25 mM. In some embodiments, the cell-free protein synthesis system comprises free nucleotides endogenous to the lysed bacteria, wherein the concentration of free nucleotides in the cell-free protein synthesis system is less than 0.125 mM. In some embodiments, the cell-free protein synthesis system comprises free nucleotides endogenous to the lysed bacteria, wherein the concentration of free nucleotides in the cell-free protein synthesis system is less than 0.1 mM. In some embodiments, the cell-free protein synthesis system comprises free nucleotides endogenous to the lysed bacteria, wherein the concentration of free nucleotides in the cell-free protein synthesis system is less than 0.075 mM. In some embodiments, the cell-free protein synthesis system comprises free nucleotides endogenous to the lysed bacteria, wherein the concentration of free nucleotides in the cell-free protein synthesis system is less than 0.05 mM. In some embodiments, the cell-free protein synthesis system comprises free nucleotides endogenous to the lysed bacteria, wherein the concentration of free nucleotides in the cell-free protein synthesis system is less than 0.025 mM. In some embodiments, provided herein is a cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the cell-free protein synthesis system comprises free nucleotides endogenous to the lysed bacteria, and wherein the concentration of free nucleotides in the cell-free protein synthesis system is less than 1.5 mM.

In some embodiments, provided herein is a cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5, wherein the cell-free protein synthesis system is maintained at a temperature less than about 30° C., wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, wherein the molecular crowder is chosen from PEG 400-8000, maltodextrin, Ficoll 70-400, dextran, and serum albumin, optionally bovine serum albumin, wherein the concentration of the molecular crowder, if present, in the cell-free protein synthesis system is from about 0 to 40 g/L, wherein the carbon source, if present, is chosen from glucose, fructose, galactose, sucrose, lactose, gluconate, starch, maltodextrin, and maltose, wherein the concentration of the carbon source, if present, in the cell-free protein synthesis system is from about 0 to 50 g/L, wherein the ammonium source, if present, comprises ammonium sulfate, wherein the concentration of the ammonium source in the cell-free protein synthesis system is from 2 to 20 mM, wherein the potassium source, if present, is chosen from potassium glutamate and potassium gluconate, wherein the concentration of the potassium glutamate, if present, in the cell-free protein synthesis system is from about 10 to 270 mM, wherein the concentration of the potassium gluconate, if present, in the cell-free protein synthesis system is from about 10 to 250 mM, wherein the magnesium source, if present, comprises magnesium glutamate, wherein the concentration of the magnesium source, if present, in the cell-free protein synthesis system is from 0 to about 10 mM, wherein the phosphate source, if present, comprises potassium phosphate, wherein the concentration of the phosphate source in the cell-free protein synthesis system is from about 5 to about 23.5 mM, wherein the sulfur source, if present, comprises ammonium sulfate, wherein the concentration of the sulfur source in the cell-free protein synthesis system is from 2 to 20 mM, wherein the buffer, if present, comprises 3-(N-morpholino)propanesulfonic acid (MOPS) buffer, wherein the concentration of the buffer in the cell-free protein synthesis system is from 50 to 300 mM, wherein the concentration of the cell-free cell extract in the cell-free protein synthesis system is from 10% to 70% (v/v), wherein the concentration of the DNA or RNA encoding the protein of interest in the cell-free protein synthesis system is from 1 to 100 μg/mL, wherein the cell-free protein synthesis system comprises free amino acids endogenous to the lysed bacteria, wherein the concentration of free amino acids in the cell-free protein synthesis system is less than 2 mM, wherein the cell-free protein synthesis system comprises free nucleotides endogenous to the lysed bacteria, and wherein the concentration of free nucleotides in the cell-free protein synthesis system is less than 1.5 mM.

In some embodiments, the cell-free protein synthesis system comprises free NADH/NADPH endogenous to the lysed bacteria, wherein the concentration of free NADH/NADPH in the cell-free protein synthesis system is less than 0.4 mM. In some embodiments, the cell-free protein synthesis system comprises free NADH/NADPH endogenous to the lysed bacteria, wherein the concentration of free NADH/NADPH in the cell-free protein synthesis system is less than 0.35 mM. In some embodiments, the cell-free protein synthesis system comprises free NADH/NADPH endogenous to the lysed bacteria, wherein the concentration of free NADH/NADPH in the cell-free protein synthesis system is less than 0.3 mM. In some embodiments, the cell-free protein synthesis system comprises free NADH/NADPH endogenous to the lysed bacteria, wherein the concentration of free NADH/NADPH in the cell-free protein synthesis system is less than 0.25 mM. In some embodiments, the cell-free protein synthesis system comprises free NADH/NADPH endogenous to the lysed bacteria, wherein the concentration of free NADH/NADPH in the cell-free protein synthesis system is less than 0.2 mM. In some embodiments, the cell-free protein synthesis system comprises free NADH/NADPH endogenous to the lysed bacteria, wherein the concentration of free NADH/NADPH in the cell-free protein synthesis system is less than 0.15 mM. In some embodiments, the cell-free protein synthesis system comprises free NADH/NADPH endogenous to the lysed bacteria, wherein the concentration of free NADH/NADPH in the cell-free protein synthesis system is less than 0.1 mM. In some embodiments, the cell-free protein synthesis system comprises free NADH/NADPH endogenous to the lysed bacteria, wherein the concentration of free NADH/NADPH in the cell-free protein synthesis system is less than 0.08 mM. In some embodiments, the cell-free protein synthesis system comprises free NADH/NADPH endogenous to the lysed bacteria, wherein the concentration of free NADH/NADPH in the cell-free protein synthesis system is less than 0.06 mM. In some embodiments, the cell-free protein synthesis system comprises free NADH/NADPH endogenous to the lysed bacteria, wherein the concentration of free NADH/NADPH in the cell-free protein synthesis system is less than 0.04 mM. In some embodiments, the cell-free protein synthesis system comprises free NADH/NADPH endogenous to the lysed bacteria, wherein the concentration of free NADH/NADPH in the cell-free protein synthesis system is less than 0.02 mM. In some embodiments, the cell-free protein synthesis system comprises free NADH/NADPH endogenous to the lysed bacteria, wherein the concentration of free NADH/NADPH in the cell-free protein synthesis system is less than 0.01 mM. In some embodiments, provided herein is a cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the cell-free protein synthesis system comprises free NADH/NADPH endogenous to the lysed bacteria, wherein the concentration of free NADH/NADPH in the cell-free protein synthesis system is less than 0.4 mM.

In some embodiments, provided herein is a cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5, wherein the cell-free protein synthesis system is maintained at a temperature less than about 30° C., wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, wherein the molecular crowder, if present, is chosen from PEG 400-8000, maltodextrin, Ficoll 70-400, dextran, and serum albumin, optionally bovine serum albumin, wherein the concentration of the molecular crowder in the cell-free protein synthesis system is from about 0 to 40 g/L, wherein the carbon source, if present, is chosen from glucose, fructose, galactose, sucrose, lactose, gluconate, starch, maltodextrin, and maltose, wherein the concentration of the carbon source in the cell-free protein synthesis system is from about 0 to 50 g/L, wherein the ammonium source, if present, comprises ammonium sulfate, wherein the concentration of the ammonium source in the cell-free protein synthesis system is from 2 to 20 mM, wherein the potassium source, if present, is chosen from potassium glutamate and potassium gluconate, wherein the concentration of the potassium glutamate in the cell-free protein synthesis system is from about 10 to 270 mM, wherein the concentration of the potassium gluconate in the cell-free protein synthesis system is from about 10 to 250 mM, wherein the magnesium source, if present, comprises magnesium glutamate, wherein the concentration of the magnesium source in the cell-free protein synthesis system is from 0 to about 10 mM, wherein the phosphate source comprises potassium phosphate, wherein the concentration of the phosphate source in the cell-free protein synthesis system is from about 5 to about 23.5 mM, wherein the sulfur source, if present, comprises ammonium sulfate, wherein the concentration of the sulfur source in the cell-free protein synthesis system is from 2 to 20 mM, wherein the buffer, if present, comprises 3-(N-morpholino)propanesulfonic acid (MOPS) buffer, wherein the concentration of the buffer in the cell-free protein synthesis system is from 50 to 300 mM, wherein the concentration of the cell-free cell extract in the cell-free protein synthesis system is from 10% to 70% (v/v), wherein the concentration of the DNA or RNA encoding the protein of interest in the cell-free protein synthesis system is from 1 to 100 μg/mL, wherein the cell-free protein synthesis system comprises free amino acids endogenous to the lysed bacteria, wherein the concentration of free amino acids in the cell-free protein synthesis system is less than 2 mM, wherein the cell-free protein synthesis system comprises free nucleotides endogenous to the lysed bacteria, wherein the concentration of free nucleotides in the cell-free protein synthesis system is less than 1.5 mM, wherein the cell-free protein synthesis system comprises free NADH/NADPH endogenous to the lysed bacteria, and wherein the concentration of free NADH/NADPH in the cell-free protein synthesis system is less than 0.4 mM.

In some embodiments, the cell-free protein synthesis system does not comprise tryptone. In some embodiments, the cell-free protein synthesis system does not comprise yeast extract. In some embodiments, provided herein is a cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, and wherein the cell-free protein synthesis system does not comprise tryptone. In some embodiments, provided herein is a cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, and wherein the cell-free protein synthesis system does not comprise yeast extract.

In some embodiments, provided herein is a cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5, wherein the cell-free protein synthesis system is maintained at a temperature less than about 30° C., wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, wherein the molecular crowder is chosen from PEG 400-8000, maltodextrin, Ficoll 70-400, dextran, and serum albumin, optionally bovine serum albumin, wherein the concentration of the molecular crowder, if present, in the cell-free protein synthesis system is from about 0 to 40 g/L, wherein the carbon source, if present, is chosen from glucose, fructose, galactose, sucrose, lactose, gluconate, starch, maltodextrin, and maltose, wherein the concentration of the carbon source in the cell-free protein synthesis system is from about 0 to 50 g/L, wherein the ammonium source, if present, comprises ammonium sulfate, wherein the concentration of the ammonium source, if present, in the cell-free protein synthesis system is from 2 to 20 mM, wherein the potassium source, if present, is chosen from potassium glutamate and potassium gluconate, wherein the concentration of the potassium glutamate in the cell-free protein synthesis system is from about 10 to 270 mM, wherein the concentration of the potassium gluconate, if present, in the cell-free protein synthesis system is from about 10 to 250 mM, wherein the magnesium source comprises magnesium glutamate, wherein the concentration of the magnesium source in the cell-free protein synthesis system is from 0 to about 10 mM, wherein the phosphate source, if present, comprises potassium phosphate, wherein the concentration of the phosphate source in the cell-free protein synthesis system is from about 5 to about 23.5 mM, wherein the sulfur source, if present, comprises ammonium sulfate, wherein the concentration of the sulfur source in the cell-free protein synthesis system is from 2 to 20 mM, wherein the buffer, if present, comprises 3-(N-morpholino)propanesulfonic acid (MOPS) buffer, wherein the concentration of the buffer in the cell-free protein synthesis system is from 50 to 300 mM, wherein the concentration of the cell-free cell extract in the cell-free protein synthesis system is from 10% to 70% (v/v), wherein the concentration of the DNA or RNA encoding the protein of interest in the cell-free protein synthesis system is from 1 to 100 μg/mL, wherein the cell-free protein synthesis system comprises free amino acids endogenous to the lysed bacteria, wherein the concentration of free amino acids in the cell-free protein synthesis system is less than 2 mM, wherein the cell-free protein synthesis system comprises free nucleotides endogenous to the lysed bacteria, wherein the concentration of free nucleotides in the cell-free protein synthesis system is less than 1.5 mM, wherein the cell-free protein synthesis system comprises free NADH/NADPH endogenous to the lysed bacteria, wherein the concentration of free NADH/NADPH in the cell-free protein synthesis system is less than 0.4 mM, wherein the cell-free protein synthesis system does not comprise tryptone, and wherein the cell-free protein synthesis system does not comprise yeast extract.

### Minimal Media

In some embodiments, provided herein is a cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria. In some embodiments, the minimal media comprises at least one of (a) a carbon source; (b) a ammonium source; (c) a magnesium source; (d) a phosphate source; (e) a sulfur source; (f) a trace mineral source; and (g) sodium succinate. In some embodiments, the minimal media comprises at least two of (a) a carbon source; (b) a ammonium source; (c) a magnesium source; (d) a phosphate source; (e) a sulfur source; (f) a trace mineral source; and (g) sodium succinate. In some embodiments, the minimal media comprises at least three of (a) a carbon source; (b) a ammonium source; (c) a magnesium source; (d) a phosphate source; (e) a sulfur source; (f) a trace mineral source; and (g) sodium succinate. In some embodiments, the minimal media comprises at least four of (a) a carbon source; (b) a ammonium source; (c) a magnesium source; (d) a phosphate source; (e) a sulfur source; (f) a trace mineral source; and (g) sodium succinate. In some embodiments, the minimal media comprises at least five of (a) a carbon source; (b) a ammonium source; (c) a magnesium source; (d) a phosphate source; (e) a sulfur source; (f) a trace mineral source; and (g) sodium succinate. In some embodiments, the minimal media comprises at least six of (a) a carbon source; (b) a ammonium source; (c) a magnesium source; (d) a phosphate source; (e) a sulfur source; (f) a trace mineral source; and (g) sodium succinate. In some embodiments, the minimal media comprises at least seven of (a) a carbon source; (b) a ammonium source; (c) a magnesium source; (d) a phosphate source; (e) a sulfur source; (f) a trace mineral source; and (g) sodium succinate. In some embodiments, the minimal media comprises (a) a carbon source; (b) a ammonium source; (c) a magnesium source; (d) a phosphate source; (e) a sulfur source; (f) a trace mineral source; and (g) sodium succinate. In some embodiments, provided herein is a cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, and wherein the minimal media comprises at least one of (a) a carbon source; (b) a ammonium source; (c) a magnesium source; (d) a phosphate source; (e) a sulfur source; (f) a trace mineral source; and (g) sodium succinate.

In some embodiments, provided herein is a cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5, wherein the cell-free protein synthesis system is maintained at a temperature less than about 30° C., wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, wherein the molecular crowder is chosen from PEG 400-8000, maltodextrin, Ficoll 70-400, dextran, and serum albumin, optionally bovine serum albumin, wherein the concentration of the molecular crowder, if present, in the cell-free protein synthesis system is from about 0 to 40 g/L, wherein the carbon source, if present, is chosen from glucose, fructose, galactose, sucrose, lactose, gluconate, starch, maltodextrin, and maltose, wherein the concentration of the carbon source in the cell-free protein synthesis system is from about 0 to 50 g/L, wherein the ammonium source, if present, comprises ammonium sulfate, wherein the concentration of the ammonium source in the cell-free protein synthesis system is from 2 to 20 mM, wherein the potassium source, if present, is chosen from potassium glutamate and potassium gluconate, wherein the concentration of the potassium glutamate in the cell-free protein synthesis system is from about 10 to 270 mM, wherein the concentration of the potassium gluconate in the cell-free protein synthesis system is from about 10 to 250 mM, wherein the magnesium source, if present, comprises magnesium glutamate, wherein the concentration of the magnesium source in the cell-free protein synthesis system is from 0 to about 10 mM, wherein the phosphate source, if present, comprises potassium phosphate, wherein the concentration of the phosphate source in the cell-free protein synthesis system is from about 5 to about 23.5 mM, wherein the sulfur source, if present, comprises ammonium sulfate, wherein the concentration of the sulfur source in the cell-free protein synthesis system is from 2 to 20 mM, wherein the buffer, if present, comprises 3-(N-morpholino)propanesulfonic acid (MOPS) buffer, wherein the concentration of the buffer in the cell-free protein synthesis system is from 50 to 300 mM, wherein the concentration of the cell-free cell extract in the cell-free protein synthesis system is from 10% to 70% (v/v), wherein the concentration of the DNA or RNA encoding the protein of interest in the cell-free protein synthesis system is from 1 to 100 μg/mL, wherein the cell-free protein synthesis system comprises free amino acids endogenous to the lysed bacteria, wherein the concentration of free amino acids in the cell-free protein synthesis system is less than 2 mM, wherein the cell-free protein synthesis system comprises free nucleotides endogenous to the lysed bacteria, wherein the concentration of free nucleotides in the cell-free protein synthesis system is less than 1.5 mM, wherein the cell-free protein synthesis system comprises free NADH/NADPH endogenous to the lysed bacteria, wherein the concentration of free NADH/NADPH in the cell-free protein synthesis system is less than 0.4 mM, wherein the cell-free protein synthesis system does not comprise tryptone, wherein the cell-free protein synthesis system does not comprise yeast extract, and wherein the minimal media comprises at least one of (a) a carbon source; (b) a ammonium source; (c) a magnesium source; (d) a phosphate source; (e) a sulfur source; (f) a trace mineral source; and (g) sodium succinate.

In some embodiments, the carbon source is chosen from D-glucose and glycerol. In some embodiments, the concentration of the D-glucose in the minimal media is from 0.1% to 2.5% (w/v). In some embodiments, the concentration of the D-glucose in the minimal media is from 0.2% to 2% (w/v). In some embodiments, the concentration of the D-glucose in the minimal media is from 0.3% to 1.5% (w/v). In some embodiments, the concentration of the D-glucose in the minimal media is from 0.4% to 1% (w/v). In some embodiments, the concentration of the D-glucose in the minimal media is from 0.4% to 0.8% (w/v). In some embodiments, the concentration of the D-glucose in the minimal media is from 0.4% to 0.6% (w/v). In some embodiments, the concentration of the D-glucose in the minimal media is 0.5% (w/v). In some embodiments, provided herein is a cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the minimal media comprises at least one of (a) a carbon source; (b) a ammonium source; (c) a magnesium source; (d) a phosphate source; (e) a sulfur source; (f) a trace mineral source; and (g) sodium succinate, wherein the carbon source is chosen from D-glucose and glycerol, and wherein the concentration of the D-glucose in the minimal media is from 0.10% to 2.5% (w/v).

In some embodiments, provided herein is a cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5, wherein the cell-free protein synthesis system is maintained at a temperature less than about 30° C., wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, wherein the molecular crowder, if present, is chosen from PEG 400-8000, maltodextrin, Ficoll 70-400, dextran, and serum albumin, optionally bovine serum albumin, wherein the concentration of the molecular crowder, if present, in the cell-free protein synthesis system is from about 0 to 40 g/L, wherein the carbon source, if present, is chosen from glucose, fructose, galactose, sucrose, lactose, gluconate, starch, maltodextrin, and maltose, wherein the concentration of the carbon source in the cell-free protein synthesis system is from about 0 to 50 g/L, wherein the ammonium source, if present, comprises ammonium sulfate, wherein the concentration of the ammonium source, if present, in the cell-free protein synthesis system is from 2 to 20 mM, wherein the potassium source, if present, is chosen from potassium glutamate and potassium gluconate, wherein the concentration of the potassium glutamate in the cell-free protein synthesis system is from about 10 to 270 mM, wherein the concentration of the potassium gluconate in the cell-free protein synthesis system is from about 10 to 250 mM, wherein the magnesium source, if present, comprises magnesium glutamate, wherein the concentration of the magnesium source in the cell-free protein synthesis system is from 0 to about 10 mM, wherein the phosphate source, if present, comprises potassium phosphate, wherein the concentration of the phosphate source in the cell-free protein synthesis system is from about 5 to about 23.5 mM, wherein the sulfur source comprises ammonium sulfate, wherein the concentration of the sulfur source in the cell-free protein synthesis system is from 2 to 20 mM, wherein the buffer, if present, comprises 3-(N-morpholino)propanesulfonic acid (MOPS) buffer, wherein the concentration of the buffer in the cell-free protein synthesis system is from 50 to 300 mM, wherein the concentration of the cell-free cell extract in the cell-free protein synthesis system is from 10% to 70% (v/v), wherein the concentration of the DNA or RNA encoding the protein of interest in the cell-free protein synthesis system is from 1 to 100 μg/mL, wherein the cell-free protein synthesis system comprises free amino acids endogenous to the lysed bacteria, wherein the concentration of free amino acids in the cell-free protein synthesis system is less than 2 mM, wherein the cell-free protein synthesis system comprises free nucleotides endogenous to the lysed bacteria, wherein the concentration of free nucleotides in the cell-free protein synthesis system is less than 1.5 mM, wherein the cell-free protein synthesis system comprises free NADH/NADPH endogenous to the lysed bacteria, wherein the concentration of free NADH/NADPH in the cell-free protein synthesis system is less than 0.4 mM, wherein the cell-free protein synthesis system does not comprise tryptone, wherein the cell-free protein synthesis system does not comprise yeast extract, wherein the minimal media comprises at least one of (a) a carbon source; (b) a ammonium source; (c) a magnesium source; (d) a phosphate source; (e) a sulfur source; (f) a trace mineral source; and (g) sodium succinate, wherein the carbon source is chosen from D-glucose and glycerol, and wherein the concentration of the D-glucose in the minimal media is from 0.1% to 2.5% (w/v).

In some embodiments, the carbon source is chosen from D-glucose and glycerol. In some embodiments, the concentration of the glycerol in the minimal media is from 0.25% to 25% (w/v). In some embodiments, the concentration of the glycerol in the minimal media is from 0.5% to 22.5% (w/v). In some embodiments, the concentration of the glycerol in the minimal media is from 0.75% to 20% (w/v). In some embodiments, the concentration of the glycerol in the minimal media is from 1% to 17.5% (w/v). In some embodiments, the concentration of the glycerol in the minimal media is from 1.25% to 15% (w/v). In some embodiments, the concentration of the glycerol in the minimal media is from 1.5% to 12.5% (w/v). In some embodiments, the concentration of the glycerol in the minimal media is from 1.75% to 10% (w/v). In some embodiments, the concentration of the glycerol in the minimal media is from 2% to 7.5% (w/v). In some embodiments, the concentration of the glycerol in the minimal media is from 2.25% to 5% (w/v). In some embodiments, the concentration of the glycerol in the minimal media is from 2.25% to 3.75% (w/v). In some embodiments, the concentration of the glycerol in the minimal media is from 2.25% to 2.75% (w/v). In some embodiments, the concentration of the glycerol in the minimal media is 2.5% (w/v). In some embodiments, provided herein is a cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the minimal media comprises at least one of (a) a carbon source; (b) a ammonium source; (c) a magnesium source; (d) a phosphate source; (e) a sulfur source; (f) a trace mineral source; and (g) sodium succinate, wherein the carbon source is chosen from D-glucose and glycerol, and wherein the concentration of the glycerol in the minimal media is from 0.25% to 25% (w/v).

In some embodiments, provided herein is a cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5, wherein the cell-free protein synthesis system is maintained at a temperature less than about 30° C., wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, wherein the molecular crowder, if present, is chosen from PEG 400-8000, maltodextrin, Ficoll 70-400, dextran, and serum albumin, optionally bovine serum albumin, wherein the concentration of the molecular crowder, if present, in the cell-free protein synthesis system is from about 0 to 40 g/L, wherein the carbon source, if present, is chosen from glucose, fructose, galactose, sucrose, lactose, gluconate, starch, maltodextrin, and maltose, wherein the concentration of the carbon source in the cell-free protein synthesis system is from about 0 to 50 g/L, wherein the ammonium source, if present, comprises ammonium sulfate, wherein the concentration of the ammonium source, if present, in the cell-free protein synthesis system is from 2 to 20 mM, wherein the potassium source is chosen from potassium glutamate and potassium gluconate, wherein the concentration of the potassium glutamate, if present, in the cell-free protein synthesis system is from about 10 to 270 mM, wherein the concentration of the potassium gluconate in the cell-free protein synthesis system is from about 10 to 250 mM, wherein the magnesium source, if present, comprises magnesium glutamate, wherein the concentration of the magnesium source in the cell-free protein synthesis system is from 0 to about 10 mM, wherein the phosphate source, if present, comprises potassium phosphate, wherein the concentration of the phosphate source in the cell-free protein synthesis system is from about 5 to about 23.5 mM, wherein the sulfur source, if present, comprises ammonium sulfate, wherein the concentration of the sulfur source in the cell-free protein synthesis system is from 2 to 20 mM, wherein the buffer, if present, comprises 3-(N-morpholino)propanesulfonic acid (MOPS) buffer, wherein the concentration of the buffer in the cell-free protein synthesis system is from 50 to 300 mM, wherein the concentration of the cell-free cell extract in the cell-free protein synthesis system is from 10% to 70% (v/v), wherein the concentration of the DNA or RNA encoding a protein of interest in the cell-free protein synthesis system is from 1 to 100 μg/mL, wherein the cell-free protein synthesis system comprises free amino acids endogenous to the lysed bacteria, wherein the concentration of free amino acids in the cell-free protein synthesis system is less than 2 mM, wherein the cell-free protein synthesis system comprises free nucleotides endogenous to the lysed bacteria, wherein the concentration of free nucleotides in the cell-free protein synthesis system is less than 1.5 mM, wherein the cell-free protein synthesis system comprises free NADH/NADPH endogenous to the lysed bacteria, wherein the concentration of free NADH/NADPH in the cell-free protein synthesis system is less than 0.4 mM, wherein the cell-free protein synthesis system does not comprise tryptone, wherein the cell-free protein synthesis system does not comprise yeast extract, wherein the minimal media comprises at least one of (a) a carbon source; (b) a ammonium source; (c) a magnesium source; (d) a phosphate source; (e) a sulfur source; (f) a trace mineral source; and (g) sodium succinate, wherein the carbon source is chosen from D-glucose and glycerol, wherein the concentration of the D-glucose in the minimal media is from 0.1% to 2.5% (w/v), and wherein the concentration of the glycerol in the minimal media is from 0.25% to 25% (w/v).

In some embodiments, the concentration of the sodium succinate in the minimal media is from 0.05% to 5% (w/v). In some embodiments, the concentration of the sodium succinate in the minimal media is from 0.10% to 4% (w/v). In some embodiments, the concentration of the sodium succinate in the minimal media is from 0.15% to 3% (w/v). In some embodiments, the concentration of the sodium succinate in the minimal media is from 0.2% to 2% (w/v). In some embodiments, the concentration of the sodium succinate in the minimal media is from 0.25% to 1% (w/v). In some embodiments, the concentration of the sodium succinate in the minimal media is from 0.25% to 0.75% (w/v). In some embodiments, the concentration of the sodium succinate in the minimal media is from 0.25% to 0.5% (w/v). In some embodiments, the concentration of the sodium succinate in the minimal media is 0.375% (w/v). In some embodiments, provided herein is a cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the minimal media comprises at least one of (a) a carbon source; (b) a ammonium source; (c) a magnesium source; (d) a phosphate source; (e) a sulfur source; (f) a trace mineral source; and (g) sodium succinate, and wherein the concentration of the sodium succinate in the minimal media is from 0.05% to 5% (w/v).

In some embodiments, provided herein is a cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5, wherein the cell-free protein synthesis system is maintained at a temperature less than about 30° C., wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, wherein the molecular crowder is chosen from PEG 400-8000, maltodextrin, Ficoll 70-400, dextran, and serum albumin, optionally bovine serum albumin, wherein the concentration of the molecular crowder, if present, in the cell-free protein synthesis system is from about 0 to 40 g/L, wherein the carbon source, if present, is chosen from glucose, fructose, galactose, sucrose, lactose, gluconate, starch, maltodextrin, and maltose, wherein the concentration of the carbon source in the cell-free protein synthesis system is from about 0 to 50 g/L, wherein the ammonium source, if present, comprises ammonium sulfate, wherein the concentration of the ammonium source in the cell-free protein synthesis system is from 2 to 20 mM, wherein the potassium source, if present, is chosen from potassium glutamate and potassium gluconate, wherein the concentration of the potassium glutamate, if present, in the cell-free protein synthesis system is from about 10 to 270 mM, wherein the concentration of the potassium gluconate, if present, in the cell-free protein synthesis system is from about 10 to 250 mM, wherein the magnesium source, if present, comprises magnesium glutamate, wherein the concentration of the magnesium source in the cell-free protein synthesis system is from 0 to about 10 mM, wherein the phosphate source, if present, comprises potassium phosphate, wherein the concentration of the phosphate source in the cell-free protein synthesis system is from about 5 to about 23.5 mM, wherein the sulfur source, if present, comprises ammonium sulfate, wherein the concentration of the sulfur source in the cell-free protein synthesis system is from 2 to 20 mM, wherein the buffer, if present, comprises 3-(N-morpholino)propanesulfonic acid (MOPS) buffer, wherein the concentration of the buffer in the cell-free protein synthesis system is from 50 to 300 mM, wherein the concentration of the cell-free cell extract in the cell-free protein synthesis system is from 10% to 70% (v/v), wherein the concentration of the DNA or RNA encoding the protein of interest in the cell-free protein synthesis system is from 1 to 100 μg/mL, wherein the cell-free protein synthesis system comprises free amino acids endogenous to the lysed bacteria, wherein the concentration of free amino acids in the cell-free protein synthesis system is less than 2 mM, wherein the cell-free protein synthesis system comprises free nucleotides endogenous to the lysed bacteria, wherein the concentration of free nucleotides in the cell-free protein synthesis system is less than 1.5 mM, wherein the cell-free protein synthesis system comprises free NADH/NADPH endogenous to the lysed bacteria, wherein the concentration of free NADH/NADPH in the cell-free protein synthesis system is less than 0.4 mM, wherein the cell-free protein synthesis system does not comprise tryptone, wherein the cell-free protein synthesis system does not comprise yeast extract, wherein the minimal media comprises at least one of (a) a carbon source; (b) a ammonium source; (c) a magnesium source; (d) a phosphate source; (e) a sulfur source; (f) a trace mineral source; and (g) sodium succinate, wherein the carbon source is chosen from D-glucose and glycerol, wherein the concentration of the D-glucose in the minimal media is from 0.1% to 2.5% (w/v), wherein the concentration of the glycerol in the minimal media is from 0.25% to 25% (w/v), and wherein the concentration of the sodium succinate in the minimal media is from 0.05% to 5% (w/v).

In some embodiments, the phosphate source comprises a sodium phosphate buffer or a potassium phosphate buffer. In some embodiments, the concentration of the potassium phosphate buffer in the minimal media is from 5 to 250 mM. In some embodiments, the concentration of the potassium phosphate buffer in the minimal media is from 10 to 200 mM. In some embodiments, the concentration of the potassium phosphate buffer in the minimal media is from 15 to 150 mM. In some embodiments, the concentration of the potassium phosphate buffer in the minimal media is from 20 to 100 mM. In some embodiments, the concentration of the potassium phosphate buffer in the minimal media is from 25 to 75 mM. In some embodiments, the concentration of the potassium phosphate buffer in the minimal media is from 37.5 to 62.5 mM. In some embodiments, the concentration of the potassium phosphate buffer in the minimal media is from 45 to 55 mM. In some embodiments, the concentration of the potassium phosphate buffer in the minimal media is from 47.5 to 52.5 mM. In some embodiments, the concentration of the potassium phosphate buffer in the minimal media is 50 mM. In some embodiments, provided herein is a cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the minimal media comprises at least one of (a) a carbon source; (b) a ammonium source; (c) a magnesium source; (d) a phosphate source; (e) a sulfur source; (f) a trace mineral source; and (g) sodium succinate, wherein the phosphate source comprises a sodium phosphate buffer or a potassium phosphate buffer, and wherein the concentration of the potassium phosphate buffer in the minimal media is from 5 to 250 mM.

In some embodiments, provided herein is a cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5, wherein the cell-free protein synthesis system is maintained at a temperature less than about 30° C., wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, wherein the molecular crowder is chosen from PEG 400-8000, maltodextrin, Ficoll 70-400, dextran, and serum albumin, optionally bovine serum albumin, wherein the concentration of the molecular crowder, if present, in the cell-free protein synthesis system is from about 0 to 40 g/L, wherein the carbon source, if present, is chosen from glucose, fructose, galactose, sucrose, lactose, gluconate, starch, maltodextrin, and maltose, wherein the concentration of the carbon source in the cell-free protein synthesis system is from about 0 to 50 g/L, wherein the ammonium source, if present, comprises ammonium sulfate, wherein the concentration of the ammonium source in the cell-free protein synthesis system is from 2 to 20 mM, wherein the potassium source, if present, is chosen from potassium glutamate and potassium gluconate, wherein the concentration of the potassium glutamate in the cell-free protein synthesis system is from about 10 to 270 mM, wherein the concentration of the potassium gluconate in the cell-free protein synthesis system is from about 10 to 250 mM, wherein the magnesium source, if present, comprises magnesium glutamate, wherein the concentration of the magnesium source in the cell-free protein synthesis system is from 0 to about 10 mM, wherein the phosphate source, if present, comprises potassium phosphate, wherein the concentration of the phosphate source in the cell-free protein synthesis system is from about 5 to about 23.5 mM, wherein the sulfur source, if present, comprises ammonium sulfate, wherein the concentration of the sulfur source in the cell-free protein synthesis system is from 2 to 20 mM, wherein the buffer, if present, comprises 3-(N-morpholino)propanesulfonic acid (MOPS) buffer, wherein the concentration of the buffer in the cell-free protein synthesis system is from 50 to 300 mM, wherein the concentration of the cell-free cell extract in the cell-free protein synthesis system is from 10% to 70% (v/v), wherein the concentration of the DNA or RNA encoding the protein of interest in the cell-free protein synthesis system is from 1 to 100 μg/mL, wherein the cell-free protein synthesis system comprises free amino acids endogenous to the lysed bacteria, wherein the concentration of free amino acids in the cell-free protein synthesis system is less than 2 mM, wherein the cell-free protein synthesis system comprises free nucleotides endogenous to the lysed bacteria, wherein the concentration of free nucleotides in the cell-free protein synthesis system is less than 1.5 mM, wherein the cell-free protein synthesis system comprises free NADH/NADPH endogenous to the lysed bacteria, wherein the concentration of free NADH/NADPH in the cell-free protein synthesis system is less than 0.4 mM, wherein the cell-free protein synthesis system does not comprise tryptone, wherein the cell-free protein synthesis system does not comprise yeast extract, wherein the minimal media comprises at least one of (a) a carbon source; (b) a ammonium source; (c) a magnesium source; (d) a phosphate source; (e) a sulfur source; (f) a trace mineral source; and (g) sodium succinate, wherein the carbon source is chosen from D-glucose and glycerol, wherein the concentration of the D-glucose in the minimal media is from 0.1% to 2.5% (w/v), wherein the concentration of the glycerol in the minimal media is from 0.25% to 25% (w/v), wherein the concentration of the sodium succinate in the minimal media is from 0.05% to 5% (w/v), wherein the phosphate source comprises a sodium phosphate buffer or a potassium phosphate buffer, and wherein the concentration of the potassium phosphate buffer in the minimal media is from 5 to 250 mM.

In some embodiments, the pH of the potassium phosphate buffer in the minimal media is from 6.7 to 7.7. In some embodiments, the pH of the potassium phosphate buffer in the minimal media is from 6.8 to 7.6. In some embodiments, the pH of the potassium phosphate buffer in the minimal media is from 6.9 to 7.5. In some embodiments, the pH of the potassium phosphate buffer in the minimal media is from 7 to 7.4. In some embodiments, the pH of the potassium phosphate buffer in the minimal media is from 7.1 to 7.3. In some embodiments, the pH of the potassium phosphate buffer in the minimal media is 7.2. In some embodiments, provided herein is a cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the minimal media comprises at least one of (a) a carbon source; (b) a ammonium source; (c) a magnesium source; (d) a phosphate source; (e) a sulfur source; (f) a trace mineral source; and (g) sodium succinate, wherein the phosphate source comprises a sodium phosphate buffer or a potassium phosphate buffer, and wherein the pH of the potassium phosphate buffer in the minimal media is from 6.7 to 7.7.

In some embodiments, provided herein is a cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5, wherein the cell-free protein synthesis system is maintained at a temperature less than about 30° C., wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, wherein the molecular crowder is chosen from PEG 400-8000, maltodextrin, Ficoll 70-400, dextran, and serum albumin, optionally bovine serum albumin, wherein the concentration of the molecular crowder, if present, in the cell-free protein synthesis system is from about 0 to 40 g/L, wherein the carbon source, if present, is chosen from glucose, fructose, galactose, sucrose, lactose, gluconate, starch, maltodextrin, and maltose, wherein the concentration of the carbon source in the cell-free protein synthesis system is from about 0 to 50 g/L, wherein the ammonium source, if present, comprises ammonium sulfate, wherein the concentration of the ammonium source in the cell-free protein synthesis system is from 2 to 20 mM, wherein the potassium source, if present, is chosen from potassium glutamate and potassium gluconate, wherein the concentration of the potassium glutamate in the cell-free protein synthesis system is from about 10 to 270 mM, wherein the concentration of the potassium gluconate in the cell-free protein synthesis system is from about 10 to 250 mM, wherein the magnesium source, if present, comprises magnesium glutamate, wherein the concentration of the magnesium source in the cell-free protein synthesis system is from 0 to about 10 mM, wherein the phosphate source comprises potassium phosphate, wherein the concentration of the phosphate source in the cell-free protein synthesis system is from about 5 to about 23.5 mM, wherein the sulfur source, if present, comprises ammonium sulfate, wherein the concentration of the sulfur source in the cell-free protein synthesis system is from 2 to 20 mM, wherein the buffer, if present, comprises 3-(N-morpholino)propanesulfonic acid (MOPS) buffer, wherein the concentration of the buffer in the cell-free protein synthesis system is from 50 to 300 mM, wherein the concentration of the cell-free cell extract in the cell-free protein synthesis system is from 10% to 70% (v/v), wherein the concentration of the DNA or RNA encoding the protein of interest in the cell-free protein synthesis system is from 1 to 100 μg/mL, wherein the cell-free protein synthesis system comprises free amino acids endogenous to the lysed bacteria, wherein the concentration of free amino acids in the cell-free protein synthesis system is less than 2 mM, wherein the cell-free protein synthesis system comprises free nucleotides endogenous to the lysed bacteria, wherein the concentration of free nucleotides in the cell-free protein synthesis system is less than 1.5 mM, wherein the cell-free protein synthesis system comprises free NADH/NADPH endogenous to the lysed bacteria, wherein the concentration of free NADH/NADPH in the cell-free protein synthesis system is less than 0.4 mM, wherein the cell-free protein synthesis system does not comprise tryptone, wherein the cell-free protein synthesis system does not comprise yeast extract, wherein the minimal media comprises at least one of (a) a carbon source; (b) a ammonium source; (c) a magnesium source; (d) a phosphate source; (e) a sulfur source; (f) a trace mineral source; and (g) sodium succinate, wherein the carbon source is chosen from D-glucose and glycerol, wherein the concentration of the D-glucose in the minimal media is from 0.1% to 2.5% (w/v), wherein the concentration of the glycerol in the minimal media is from 0.25% to 25% (w/v), wherein the concentration of the sodium succinate in the minimal media is from 0.05% to 5% (w/v), wherein the phosphate source comprises a sodium phosphate buffer or a potassium phosphate buffer, wherein the concentration of the potassium phosphate buffer in the minimal media is from 5 to 250 mM, and wherein the pH of the potassium phosphate buffer in the minimal media is from 6.7 to 7.7.

In some embodiments, the ammonium source comprises ammonium chloride. In some embodiments, the concentration of the ammonium chloride in the minimal media is from 5 to 250 mM. In some embodiments, the concentration of the ammonium chloride in the minimal media is from 10 to 200 mM. In some embodiments, the concentration of the ammonium chloride in the minimal media is from 15 to 150 mM. In some embodiments, the concentration of the ammonium chloride in the minimal media is from 20 to 100 mM. In some embodiments, the concentration of the ammonium chloride in the minimal media is from 25 to 75 mM. In some embodiments, the concentration of the ammonium chloride in the minimal media is from 37.5 to 62.5 mM. In some embodiments, the concentration of the ammonium chloride in the minimal media is from 45 to 55 mM. In some embodiments, the concentration of the ammonium chloride in the minimal media is from 47.5 to 52.5 mM. In some embodiments, the concentration of the ammonium chloride in the minimal media is 50 mM. In some embodiments, provided herein is a cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the minimal media comprises at least one of (a) a carbon source; (b) a ammonium source; (c) a magnesium source; (d) a phosphate source; (e) a sulfur source; (f) a trace mineral source; and (g) sodium succinate, wherein the ammonium source comprises ammonium chloride, and wherein the concentration of the ammonium chloride in the minimal media is from 5 to 250 mM.

In some embodiments, provided herein is a cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5, wherein the cell-free protein synthesis system is maintained at a temperature less than about 30° C., wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, wherein the molecular crowder is chosen from PEG 400-8000, maltodextrin, Ficoll 70-400, dextran, and serum albumin, optionally bovine serum albumin, wherein the concentration of the molecular crowder, if present, in the cell-free protein synthesis system is from about 0 to 40 g/L, wherein the carbon source, if present, is chosen from glucose, fructose, galactose, sucrose, lactose, gluconate, starch, maltodextrin, and maltose, wherein the concentration of the carbon source in the cell-free protein synthesis system is from about 0 to 50 g/L, wherein the ammonium source, if present, comprises ammonium sulfate, wherein the concentration of the ammonium source, if present, in the cell-free protein synthesis system is from 2 to 20 mM, wherein the potassium source, if present, is chosen from potassium glutamate and potassium gluconate, wherein the concentration of the potassium glutamate in the cell-free protein synthesis system is from about 10 to 270 mM, wherein the concentration of the potassium gluconate in the cell-free protein synthesis system is from about 10 to 250 mM, wherein the magnesium source, if present, comprises magnesium glutamate, wherein the concentration of the magnesium source in the cell-free protein synthesis system is from 0 to about 10 mM, wherein the phosphate source, if present, comprises potassium phosphate, wherein the concentration of the phosphate source in the cell-free protein synthesis system is from about 5 to about 23.5 mM, wherein the sulfur source comprises ammonium sulfate, wherein the concentration of the sulfur source in the cell-free protein synthesis system is from 2 to 20 mM, wherein the buffer comprises 3-(N-morpholino)propanesulfonic acid (MOPS) buffer, wherein the concentration of the buffer in the cell-free protein synthesis system is from 50 to 300 mM, wherein the concentration of the cell-free cell extract in the cell-free protein synthesis system is from 10% to 70% (v/v), wherein the concentration of the DNA or RNA encoding the protein of interest in the cell-free protein synthesis system is from 1 to 100 μg/mL, wherein the cell-free protein synthesis system comprises free amino acids endogenous to the lysed bacteria, wherein the concentration of free amino acids in the cell-free protein synthesis system is less than 2 mM, wherein the cell-free protein synthesis system comprises free nucleotides endogenous to the lysed bacteria, wherein the concentration of free nucleotides in the cell-free protein synthesis system is less than 1.5 mM, wherein the cell-free protein synthesis system comprises free NADH/NADPH endogenous to the lysed bacteria, wherein the concentration of free NADH/NADPH in the cell-free protein synthesis system is less than 0.4 mM, wherein the cell-free protein synthesis system does not comprise tryptone, wherein the cell-free protein synthesis system does not comprise yeast extract, wherein the minimal media comprises at least one of (a) a carbon source; (b) a ammonium source; (c) a magnesium source; (d) a phosphate source; (e) a sulfur source; (f) a trace mineral source; and (g) sodium succinate, wherein the carbon source is chosen from D-glucose and glycerol, wherein the concentration of the D-glucose in the minimal media is from 0.1% to 2.5% (w/v), wherein the concentration of the glycerol in the minimal media is from 0.25% to 25% (w/v), wherein the concentration of the sodium succinate in the minimal media is from 0.05% to 5% (w/v), wherein the phosphate source comprises a sodium phosphate buffer or a potassium phosphate buffer, wherein the concentration of the potassium phosphate buffer in the minimal media is from 5 to 250 mM, wherein the pH of the potassium phosphate buffer in the minimal media is from 6.7 to 7.7, wherein the ammonium source comprises ammonium chloride, and wherein the concentration of the ammonium chloride in the minimal media is from 5 to 250 mM.

In some embodiments, sulfur source comprises sodium sulfate. In some embodiments, the concentration of the sodium sulfate in the minimal media is from 0.5 to 25 mM. In some embodiments, the concentration of the sodium sulfate in the minimal media is from 1 to 20 mM. In some embodiments, the concentration of the sodium sulfate in the minimal media is from 1.5 to 15 mM. In some embodiments, the concentration of the sodium sulfate in the minimal media is from 2 to 10 mM. In some embodiments, the concentration of the sodium sulfate in the minimal media is from 2.5 to 7.5 mM. In some embodiments, the concentration of the sodium sulfate in the minimal media is from 3.75 to 6.25 mM. In some embodiments, the concentration of the sodium sulfate in the minimal media is from 4.5 to 5.5 mM. In some embodiments, the concentration of the sodium sulfate in the minimal media is from 4.75 to 5.25 mM. In some embodiments, the concentration of the sodium sulfate in the minimal media is 5 mM. In some embodiments, provided herein is a cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the minimal media comprises at least one of (a) a carbon source; (b) a ammonium source; (c) a magnesium source; (d) a phosphate source; (e) a sulfur source; (f) a trace mineral source; and (g) sodium succinate, wherein the sulfur source comprises sodium sulfate, and wherein the concentration of the sodium sulfate in the minimal media is from 0.5 to 25 mM.

In some embodiments, provided herein is a cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5, wherein the cell-free protein synthesis system is maintained at a temperature less than about 30° C., wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, wherein the molecular crowder is chosen from PEG 400-8000, maltodextrin, Ficoll 70-400, dextran, and serum albumin, optionally bovine serum albumin, wherein the concentration of the molecular crowder, if present, in the cell-free protein synthesis system is from about 0 to 40 g/L, wherein the carbon source, if present, is chosen from glucose, fructose, galactose, sucrose, lactose, gluconate, starch, maltodextrin, and maltose, wherein the concentration of the carbon source in the cell-free protein synthesis system is from about 0 to 50 g/L, wherein the ammonium source, if present, comprises ammonium sulfate, wherein the concentration of the ammonium source in the cell-free protein synthesis system is from 2 to 20 mM, wherein the potassium source, if present, is chosen from potassium glutamate and potassium gluconate, wherein the concentration of the potassium glutamate in the cell-free protein synthesis system is from about 10 to 270 mM, wherein the concentration of the potassium gluconate in the cell-free protein synthesis system is from about 10 to 250 mM, wherein the magnesium source, if present, comprises magnesium glutamate, wherein the concentration of the magnesium source in the cell-free protein synthesis system is from 0 to about 10 mM, wherein the phosphate source, if present, comprises potassium phosphate, wherein the concentration of the phosphate source in the cell-free protein synthesis system is from about 5 to about 23.5 mM, wherein the sulfur source comprises ammonium sulfate, wherein the concentration of the sulfur source in the cell-free protein synthesis system is from 2 to 20 mM, wherein the buffer comprises 3-(N-morpholino)propanesulfonic acid (MOPS) buffer, wherein the concentration of the buffer in the cell-free protein synthesis system is from 50 to 300 mM, wherein the concentration of the cell-free cell extract in the cell-free protein synthesis system is from 10% to 70% (v/v), wherein the concentration of the DNA or RNA encoding the protein of interest in the cell-free protein synthesis system is from 1 to 100 μg/mL, wherein the cell-free protein synthesis system comprises free amino acids endogenous to the lysed bacteria, wherein the concentration of free amino acids in the cell-free protein synthesis system is less than 2 mM, wherein the cell-free protein synthesis system comprises free nucleotides endogenous to the lysed bacteria, wherein the concentration of free nucleotides in the cell-free protein synthesis system is less than 1.5 mM, wherein the cell-free protein synthesis system comprises free NADH/NADPH endogenous to the lysed bacteria, wherein the concentration of free NADH/NADPH in the cell-free protein synthesis system is less than 0.4 mM, wherein the cell-free protein synthesis system does not comprise tryptone, wherein the cell-free protein synthesis system does not comprise yeast extract, wherein the minimal media comprises at least one of (a) a carbon source; (b) a ammonium source; (c) a magnesium source; (d) a phosphate source; (e) a sulfur source; (f) a trace mineral source; and (g) sodium succinate, wherein the carbon source is chosen from D-glucose and glycerol, wherein the concentration of the D-glucose in the minimal media is from 0.1% to 2.5% (w/v), wherein the concentration of the glycerol in the minimal media is from 0.25% to 25% (w/v), wherein the concentration of the sodium succinate in the minimal media is from 0.05% to 5% (w/v), wherein the phosphate source comprises a sodium phosphate buffer or a potassium phosphate buffer, wherein the concentration of the potassium phosphate buffer in the minimal media is from 5 to 250 mM, wherein the pH of the potassium phosphate buffer in the minimal media is from 6.7 to 7.7, wherein the ammonium source comprises ammonium chloride, wherein the concentration of the ammonium chloride in the minimal media is from 5 to 250 mM, wherein the sulfur source comprises sodium sulfate, and wherein the concentration of the sodium sulfate in the minimal media is from 0.5 to 25 mM.

In some embodiments, the magnesium source comprises magnesium sulfate heptahydrate. In some embodiments, the concentration of the magnesium sulfate heptahydrate in the minimal media is from 0.2 to 20 mM. In some embodiments, the concentration of the magnesium sulfate heptahydrate in the minimal media is from 0.3 to 19 mM. In some embodiments, the concentration of the magnesium sulfate heptahydrate in the minimal media is from 0.4 to 18 mM. In some embodiments, the concentration of the magnesium sulfate heptahydrate in the minimal media is from 0.5 to 17 mM. In some embodiments, the concentration of the magnesium sulfate heptahydrate in the minimal media is from 0.6 to 16 mM. In some embodiments, the concentration of the magnesium sulfate heptahydrate in the minimal media is from 0.7 to 15 mM. In some embodiments, the concentration of the magnesium sulfate heptahydrate in the minimal media is from 0.8 to 14 mM. In some embodiments, the concentration of the magnesium sulfate heptahydrate in the minimal media is from 0.9 to 13 mM. In some embodiments, the concentration of the magnesium sulfate heptahydrate in the minimal media is from 1 to 12 mM. In some embodiments, the concentration of the magnesium sulfate heptahydrate in the minimal media is from 1.1 to 11 mM. In some embodiments, the concentration of the magnesium sulfate heptahydrate in the minimal media is from 1.2 to 10 mM. In some embodiments, the concentration of the magnesium sulfate heptahydrate in the minimal media is from 1.3 to 9 mM. In some embodiments, the concentration of the magnesium sulfate heptahydrate in the minimal media is from 1.4 to 8 mM. In some embodiments, the concentration of the magnesium sulfate heptahydrate in the minimal media is from 1.5 to 7 mM. In some embodiments, the concentration of the magnesium sulfate heptahydrate in the minimal media is from 1.6 to 6 mM. In some embodiments, the concentration of the magnesium sulfate heptahydrate in the minimal media is from 1.7 to 5 mM. In some embodiments, the concentration of the magnesium sulfate heptahydrate in the minimal media is from 1.8 to 4 mM. In some embodiments, the concentration of the magnesium sulfate heptahydrate in the minimal media is from 1.8 to 3 mM. In some embodiments, the concentration of the magnesium sulfate heptahydrate in the minimal media is from 1.9 to 2.5 mM. In some embodiments, the concentration of the magnesium sulfate heptahydrate in the minimal media is from 1.9 to 2.25 mM. In some embodiments, the concentration of the magnesium sulfate heptahydrate in the minimal media is from 1.9 to 2.1 mM. In some embodiments, the concentration of the magnesium sulfate heptahydrate in the minimal media is 2 mM. In some embodiments, provided herein is a cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the minimal media comprises at least one of (a) a carbon source; (b) a ammonium source; (c) a magnesium source; (d) a phosphate source; (e) a sulfur source; (f) a trace mineral source; and (g) sodium succinate, wherein the magnesium source comprises magnesium sulfate heptahydrate, and wherein the concentration of the magnesium sulfate heptahydrate in the minimal media is from 0.2 to 20 mM.

In some embodiments, provided herein is a cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5, wherein the cell-free protein synthesis system is maintained at a temperature less than about 30° C., wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, wherein the molecular crowder is chosen from PEG 400-8000, maltodextrin, Ficoll 70-400, dextran, and serum albumin, optionally bovine serum albumin, wherein the concentration of the molecular crowder, if present, in the cell-free protein synthesis system is from about 0 to 40 g/L, wherein the carbon source, if present, is chosen from glucose, fructose, galactose, sucrose, lactose, gluconate, starch, maltodextrin, and maltose, wherein the concentration of the carbon source in the cell-free protein synthesis system is from about 0 to 50 g/L, wherein the ammonium source, if present, comprises ammonium sulfate, wherein the concentration of the ammonium source in the cell-free protein synthesis system is from 2 to 20 mM, wherein the potassium source, if present, is chosen from potassium glutamate and potassium gluconate, wherein the concentration of the potassium glutamate in the cell-free protein synthesis system is from about 10 to 270 mM, wherein the concentration of the potassium gluconate in the cell-free protein synthesis system is from about 10 to 250 mM, wherein the magnesium source, if present, comprises magnesium glutamate, wherein the concentration of the magnesium source in the cell-free protein synthesis system is from 0 to about 10 mM, wherein the phosphate source, if present, comprises potassium phosphate, wherein the concentration of the phosphate source in the cell-free protein synthesis system is from about 5 to about 23.5 mM, wherein the sulfur source comprises ammonium sulfate, wherein the concentration of the sulfur source in the cell-free protein synthesis system is from 2 to 20 mM, wherein the buffer, if present, comprises 3-(N-morpholino)propanesulfonic acid (MOPS) buffer, wherein the concentration of the buffer in the cell-free protein synthesis system is from 50 to 300 mM, wherein the concentration of the cell-free cell extract in the cell-free protein synthesis system is from 10% to 70% (v/v), wherein the concentration of the DNA or RNA encoding the protein of interest in the cell-free protein synthesis system is from 1 to 100 μg/mL, wherein the cell-free protein synthesis system comprises free amino acids endogenous to the lysed bacteria, wherein the concentration of free amino acids in the cell-free protein synthesis system is less than 2 mM, wherein the cell-free protein synthesis system comprises free nucleotides endogenous to the lysed bacteria, wherein the concentration of free nucleotides in the cell-free protein synthesis system is less than 1.5 mM, wherein the cell-free protein synthesis system comprises free NADH/NADPH endogenous to the lysed bacteria, wherein the concentration of free NADH/NADPH in the cell-free protein synthesis system is less than 0.4 mM, wherein the cell-free protein synthesis system does not comprise tryptone, wherein the cell-free protein synthesis system does not comprise yeast extract, wherein the minimal media comprises at least one of (a) a carbon source; (b) a ammonium source; (c) a magnesium source; (d) a phosphate source; (e) a sulfur source; (f) a trace mineral source; and (g) sodium succinate, wherein the carbon source is chosen from D-glucose and glycerol, wherein the concentration of the D-glucose in the minimal media is from 0.1% to 2.5% (w/v), wherein the concentration of the glycerol in the minimal media is from 0.25% to 25% (w/v), wherein the concentration of the sodium succinate in the minimal media is from 0.05% to 5% (w/v), wherein the phosphate source comprises a sodium phosphate buffer or a potassium phosphate buffer, wherein the concentration of the potassium phosphate buffer in the minimal media is from 5 to 250 mM, wherein the pH of the potassium phosphate buffer in the minimal media is from 6.7 to 7.7, wherein the ammonium source comprises ammonium chloride, wherein the concentration of the ammonium chloride in the minimal media is from 5 to 250 mM, wherein the sulfur source comprises sodium sulfate, and wherein the sulfur source comprises sodium sulfate, wherein the concentration of the sodium sulfate in the minimal media is from 0.5 to 25 mM, wherein the magnesium source comprises magnesium sulfate heptahydrate, and wherein the concentration of the magnesium sulfate heptahydrate in the minimal media is from 0.2 to 20 mM.

In some embodiments, the trace mineral source is chosen from an iron source, a copper source, a nickel source, a zinc source, a molybdenum source, a boron source, and a manganese source. In some embodiments, the iron source comprises ferric citrate. In some embodiments, the concentration of the ferric citrate in the minimal media is from 10 to 1,000 M. In some embodiments, the concentration of the ferric citrate in the minimal media is from 20 to 900 M. In some embodiments, the concentration of the ferric citrate in the minimal media is from 30 to 800 M. In some embodiments, the concentration of the ferric citrate in the minimal media is from 40 to 700 M. In some embodiments, the concentration of the ferric citrate in the minimal media is from 50 to 600 M. In some embodiments, the concentration of the ferric citrate in the minimal media is from 60 to 500 M. In some embodiments, the concentration of the ferric citrate in the minimal media is from 70 to 400 M. In some embodiments, the concentration of the ferric citrate in the minimal media is from 80 to 300 M. In some embodiments, the concentration of the ferric citrate in the minimal media is from 90 to 200 M. In some embodiments, the concentration of the ferric citrate in the minimal media is from 90 to 150 M. In some embodiments, the concentration of the ferric citrate in the minimal media is from 95 to 125 M. In some embodiments, the concentration of the ferric citrate in the minimal media is from 95 to 105 M. In some embodiments, the concentration of the ferric citrate in the minimal media is 100 M. In some embodiments, provided herein is a cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the minimal media comprises at least one of (a) a carbon source; (b) a ammonium source; (c) a magnesium source; (d) a phosphate source; (e) a sulfur source; (f) a trace mineral source; and (g) sodium succinate, wherein the trace mineral source is chosen from an iron source, a copper source, a nickel source, a zinc source, a molybdenum source, a boron source, and a manganese source, wherein the iron source comprises ferric citrate, and wherein the concentration of the ferric citrate in the minimal media is from 10 to 1,000 M.

In some embodiments, provided herein is a cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5, wherein the cell-free protein synthesis system is maintained at a temperature less than about 30° C., wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, wherein the molecular crowder is chosen from PEG 400-8000, maltodextrin, Ficoll 70-400, dextran, and serum albumin, optionally bovine serum albumin, wherein the concentration of the molecular crowder, if present, in the cell-free protein synthesis system is from about 0 to 40 g/L, wherein the carbon source, if present, is chosen from glucose, fructose, galactose, sucrose, lactose, gluconate, starch, maltodextrin, and maltose, wherein the concentration of the carbon source in the cell-free protein synthesis system is from about 0 to 50 g/L, wherein the ammonium source, if present, comprises ammonium sulfate, wherein the concentration of the ammonium source in the cell-free protein synthesis system is from 2 to 20 mM, wherein the potassium source, if present, is chosen from potassium glutamate and potassium gluconate, wherein the concentration of the potassium glutamate in the cell-free protein synthesis system is from about 10 to 270 mM, wherein the concentration of the potassium gluconate in the cell-free protein synthesis system is from about 10 to 250 mM, wherein the magnesium source, if present, comprises magnesium glutamate, wherein the concentration of the magnesium source in the cell-free protein synthesis system is from 0 to about 10 mM, wherein the phosphate source, if present, comprises potassium phosphate, wherein the concentration of the phosphate source in the cell-free protein synthesis system is from about 5 to about 23.5 mM, wherein the sulfur source, if present, comprises ammonium sulfate, wherein the concentration of the sulfur source in the cell-free protein synthesis system is from 2 to 20 mM, wherein the buffer, if present, comprises 3-(N-morpholino)propanesulfonic acid (MOPS) buffer, wherein the concentration of the buffer in the cell-free protein synthesis system is from 50 to 300 mM, wherein the concentration of the cell-free cell extract in the cell-free protein synthesis system is from 10% to 70% (v/v), wherein the concentration of the DNA or RNA encoding the protein of interest in the cell-free protein synthesis system is from 1 to 100 μg/mL, wherein the cell-free protein synthesis system comprises free amino acids endogenous to the lysed bacteria, wherein the concentration of free amino acids in the cell-free protein synthesis system is less than 2 mM, wherein the cell-free protein synthesis system comprises free nucleotides endogenous to the lysed bacteria, wherein the concentration of free nucleotides in the cell-free protein synthesis system is less than 1.5 mM, wherein the cell-free protein synthesis system comprises free NADH/NADPH endogenous to the lysed bacteria, wherein the concentration of free NADH/NADPH in the cell-free protein synthesis system is less than 0.4 mM, wherein the cell-free protein synthesis system does not comprise tryptone, wherein the cell-free protein synthesis system does not comprise yeast extract, wherein the minimal media comprises at least one of (a) a carbon source; (b) a ammonium source; (c) a magnesium source; (d) a phosphate source; (e) a sulfur source; (f) a trace mineral source; and (g) sodium succinate, wherein the carbon source is chosen from D-glucose and glycerol, wherein the concentration of the D-glucose in the minimal media is from 0.1% to 2.5% (w/v), wherein the concentration of the glycerol in the minimal media is from 0.25% to 25% (w/v), wherein the concentration of the sodium succinate in the minimal media is from 0.05% to 5% (w/v), wherein the phosphate source comprises a sodium phosphate buffer or a potassium phosphate buffer, wherein the concentration of the potassium phosphate buffer in the minimal media is from 5 to 250 mM, wherein the pH of the potassium phosphate buffer in the minimal media is from 6.7 to 7.7, wherein the ammonium source comprises ammonium chloride, wherein the concentration of the ammonium chloride in the minimal media is from 5 to 250 mM, wherein the sulfur source comprises sodium sulfate, and wherein the sulfur source comprises sodium sulfate, wherein the concentration of the sodium sulfate in the minimal media is from 0.5 to 25 mM, wherein the magnesium source comprises magnesium sulfate heptahydrate, wherein the concentration of the magnesium sulfate heptahydrate in the minimal media is from 0.2 to 20 mM, wherein the trace mineral source is chosen from an iron source, a copper source, a nickel source, a zinc source, a molybdenum source, a boron source, and a manganese source, wherein the iron source comprises ferric citrate, and wherein the concentration of the ferric citrate in the minimal media is from 10 to 1,000 μM.

### Bacterial Culture

In some embodiments, provided herein is a cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, and wherein the bacteria are Escherichia coli (E. coli), Bacillus subtilis (B. subtilis), or Vibrio natriegens (V natriegens).

In some embodiments, the bacteria are grown in any minimal media. In some embodiments, the bacteria are grown in lysogeny broth (LB) media. In some embodiments, the bacteria are pelleted (i.e., supernatant removed and bacteria spun down), and then the pellet is resuspended in minimal media at 4% (v/v), optionally wherein the minimal media further comprises about 12.5 μg/mL chloramphenicol and 50 μg/mL isopropyl β-d-1-thiogalactopyranoside (IPTG).

In some embodiments, the bacteria are cultured in minimal media at 37° C. and are monitored until an OD 600 of 7 to 10 is reached. In some embodiments, the bacteria are pelleted, and the supernatant is disposed of. In some embodiments, the bacteria are washed once with buffer at a ratio of 20 mL buffer per gram of bacteria, the buffer containing 10 mM Tris-acetate (pH 7.9), 14 mM magnesium glutamate, and 60 mM potassium glutamate. In some embodiments, the bacteria are pelleted and resuspended in buffer at a ratio of 1.1 mL buffer per gram of bacteria to yield a cell-buffer suspension. In some embodiments, the cell-buffer suspension is immersed in an ice-water bath for preparation of the cell-free cell extract.

### Cell-Free Cell Extract

In some embodiments, provided herein is a cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, and wherein the cell-free cell extract is prepared by preparing a suspension in which each one gram of the bacteria is suspended in 0.1 mL to 6.1 mL of a suspension buffer. In some embodiments, the cell-free cell extract is prepared by preparing a suspension in which each one gram of the bacteria is suspended in 0.2 mL to 5.6 mL of a suspension buffer. In some embodiments, the cell-free cell extract is prepared by preparing a suspension in which each one gram of the bacteria is suspended in 0.3 mL to 5.1 mL of a suspension buffer. In some embodiments, the cell-free cell extract is prepared by preparing a suspension in which each one gram of the bacteria is suspended in 0.4 mL to 4.6 mL of a suspension buffer. In some embodiments, the cell-free cell extract is prepared by preparing a suspension in which each one gram of the bacteria is suspended in 0.5 mL to 4.1 mL of a suspension buffer. In some embodiments, the cell-free cell extract is prepared by preparing a suspension in which each one gram of the bacteria is suspended in 0.6 mL to 3.6 mL of a suspension buffer. In some embodiments, the cell-free cell extract is prepared by preparing a suspension in which each one gram of the bacteria is suspended in 0.7 mL to 3.1 mL of a suspension buffer. In some embodiments, the cell-free cell extract is prepared by preparing a suspension in which each one gram of the bacteria is suspended in 0.8 mL to 2.6 mL of a suspension buffer. In some embodiments, the cell-free cell extract is prepared by preparing a suspension in which each one gram of the bacteria is suspended in 0.9 mL to 2.1 mL of a suspension buffer. In some embodiments, the cell-free cell extract is prepared by preparing a suspension in which each one gram of the bacteria is suspended in 1 mL to 1.6 mL of a suspension buffer. In some embodiments, the cell-free cell extract is prepared by preparing a suspension in which each one gram of the bacteria is suspended in 1.05 mL to 1.35 mL of a suspension buffer. In some embodiments, the cell-free cell extract is prepared by preparing a suspension in which each one gram of the bacteria is suspended in 1.05 mL to 1.2 mL of a suspension buffer.

In some embodiments, the cell-free cell extract is prepared by preparing a suspension in which each one gram of the bacteria is suspended in 1.1 mL of a suspension buffer. In some embodiments, provided herein is a cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5, wherein the cell-free protein synthesis system is maintained at a temperature less than about 30° C., wherein the reaction mixture further comprises at least one of: (a) a molecular crowder; (b) a carbon source; (c) a ammonium source; (d) a potassium source; € a magnesium source; (f) a phosphate source; and (g) a sulfur source; and (h) a buffer, wherein the molecular crowder is chosen from PEG 400-8000, maltodextrin, Ficoll 70-400, dextran, and serum albumin, optionally bovine serum albumin, wherein the concentration of the molecular crowder, if present, in the cell-free protein synthesis system is from about 0 to 40 g/L, wherein the carbon source, if present, is chosen from glucose, fructose, galactose, sucrose, lactose, gluconate, starch, maltodextrin, and maltose, wherein the concentration of the carbon source, if present, in the cell-free protein synthesis system is from about 0 to 50 g/L, wherein the ammonium source, if present, comprises ammonium sulfate, wherein the concentration of the ammonium source in the cell-free protein synthesis system is from 2 to 20 mM, wherein the potassium source, if present, is chosen from potassium glutamate and potassium gluconate, wherein the concentration of the potassium glutamate in the cell-free protein synthesis system is from about 10 to 270 mM, wherein the concentration of the potassium gluconate in the cell-free protein synthesis system is from about 10 to 250 mM, wherein the magnesium source, if present, comprises magnesium glutamate, wherein the concentration of the magnesium source in the cell-free protein synthesis system is from 0 to about 10 mM, wherein the phosphate source comprises potassium phosphate, wherein the concentration of the phosphate source in the cell-free protein synthesis system is from about 5 to about 23.5 mM, wherein the sulfur source comprises ammonium sulfate, wherein the concentration of the sulfur source in the cell-free protein synthesis system is from 2 to 20 mM, wherein the buffer, if present, comprises 3-(N-morpholino)propanesulfonic acid (MOPS) buffer, wherein the concentration of the buffer in the cell-free protein synthesis system is from 50 to 300 mM, wherein the concentration of the cell-free cell extract in the cell-free protein synthesis system is from 10% to 70% (v/v), wherein the concentration of the DNA or RNA encoding the protein of interest in the cell-free protein synthesis system is from 1 to 100 μg/mL, wherein the cell-free protein synthesis system comprises free amino acids endogenous to the lysed bacteria, wherein the concentration of free amino acids in the cell-free protein synthesis system is less than 2 mM, wherein the cell-free protein synthesis system comprises free nucleotides endogenous to the lysed bacteria, wherein the concentration of free nucleotides in the cell-free protein synthesis system is less than 1.5 mM, wherein the cell-free protein synthesis system comprises free NADH/NADPH endogenous to the lysed bacteria, wherein the concentration of free NADH/NADPH in the cell-free protein synthesis system is less than 0.4 mM, wherein the cell-free protein synthesis system does not comprise tryptone, wherein the cell-free protein synthesis system does not comprise yeast extract, wherein the minimal media comprises at least one of (a) a carbon source; (b) a ammonium source; (c) a magnesium source; (d) a phosphate source; (e) a sulfur source; (f) a trace mineral source; and (g) sodium succinate, wherein the carbon source is chosen from D-glucose and glycerol, wherein the concentration of the D-glucose in the minimal media is from 0.1% to 2.5% (w/v), wherein the concentration of the glycerol in the minimal media is from 0.25% to 25% (w/v), wherein the concentration of the sodium succinate in the minimal media is from 0.05% to 5% (w/v), wherein the phosphate source comprises a sodium phosphate buffer or a potassium phosphate buffer, wherein the concentration of the potassium phosphate buffer in the minimal media is from 5 to 250 mM, wherein the pH of the potassium phosphate buffer in the minimal media is from 6.7 to 7.7, wherein the ammonium source comprises ammonium chloride, wherein the concentration of the ammonium chloride in the minimal media is from 5 to 250 mM, wherein the sulfur source comprises sodium sulfate, and wherein the sulfur source comprises sodium sulfate, wherein the concentration of the sodium sulfate in the minimal media is from 0.5 to 25 mM, wherein the magnesium source comprises magnesium sulfate heptahydrate, wherein the concentration of the magnesium sulfate heptahydrate in the minimal media is from 0.2 to 20 mM, wherein the trace mineral source is chosen from an iron source, a copper source, a nickel source, a zinc source, a molybdenum source, a boron source, and a manganese source, wherein the iron source comprises ferric citrate, wherein the concentration of the ferric citrate in the minimal media is from 10 to 1,000 M, and wherein the cell-free cell extract is prepared by preparing a suspension in which each one gram of the bacteria is suspended in 0.1 mL to 6.1 mL of a suspension buffer. In some embodiments, the cell-free cell extract is prepared by lysing the bacteria in the suspension. In some embodiments, the lysing comprises sonication. In some embodiments, the sonication is performed using a 10 second on, 10 second off cycle, and 50% amplitude. In some embodiments, the sonication is performed until about 3,500 Joules in input energy is reached. In some embodiments, the bacterial lysate is centrifuged. In some embodiments, the bacterial lysate is centrifuged at 12,000 RCF at 4° C. for 15 minutes. In some embodiments, the supernatant is collected and immediately frozen at −80° C. In some embodiments, provided herein is a cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the cell-free cell extract is prepared by preparing a suspension in which each one gram of the bacteria is suspended in 0.1 mL to 6.1 mL of a suspension buffer, and wherein the cell-free cell extract is prepared by lysing the bacteria in the suspension. In some embodiments, provided herein is a cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the cell-free cell extract is prepared by preparing a suspension in which each one gram of the bacteria is suspended in 0.1 mL to 6.1 mL of a suspension buffer, wherein the cell-free cell extract is prepared by lysing the bacteria in the suspension, and wherein the lysing comprises sonication.

In some embodiments, provided herein is a cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5, wherein the cell-free protein synthesis system is maintained at a temperature less than about 30° C., wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, wherein the molecular crowder is chosen from PEG 400-8000, maltodextrin, Ficoll 70-400, dextran, and serum albumin, optionally bovine serum albumin, wherein the concentration of the molecular crowder, if present, in the cell-free protein synthesis system is from about 0 to 40 g/L, wherein the carbon source is chosen from glucose, fructose, galactose, sucrose, lactose, gluconate, starch, maltodextrin, and maltose, wherein the concentration of the carbon source, if present, in the cell-free protein synthesis system is from about 0 to 50 g/L, wherein the ammonium source, if present, comprises ammonium sulfate, wherein the concentration of the ammonium source, if present, in the cell-free protein synthesis system is from 2 to 20 mM, wherein the potassium source is chosen from potassium glutamate and potassium gluconate, wherein the concentration of the potassium glutamate in the cell-free protein synthesis system is from about 10 to 270 mM, wherein the concentration of the potassium gluconate in the cell-free protein synthesis system is from about 10 to 250 mM, wherein the magnesium source, if present, comprises magnesium glutamate, wherein the concentration of the magnesium source in the cell-free protein synthesis system is from 0 to about 10 mM, wherein the phosphate source, if present, comprises potassium phosphate, wherein the concentration of the phosphate source in the cell-free protein synthesis system is from about 5 to about 23.5 mM, wherein the sulfur source comprises ammonium sulfate, wherein the concentration of the sulfur source in the cell-free protein synthesis system is from 2 to 20 mM, wherein the buffer, if present, comprises 3-(N-morpholino)propanesulfonic acid (MOPS) buffer, wherein the concentration of the buffer in the cell-free protein synthesis system is from 50 to 300 mM, wherein the concentration of the cell-free cell extract in the cell-free protein synthesis system is from 10% to 70% (v/v), wherein the concentration of the DNA or RNA encoding the protein of interest in the cell-free protein synthesis system is from 1 to 100 μg/mL, wherein the cell-free protein synthesis system comprises free amino acids endogenous to the lysed bacteria, wherein the concentration of free amino acids in the cell-free protein synthesis system is less than 2 mM, wherein the cell-free protein synthesis system comprises free nucleotides endogenous to the lysed bacteria, wherein the concentration of free nucleotides in the cell-free protein synthesis system is less than 1.5 mM, wherein the cell-free protein synthesis system comprises free NADH/NADPH endogenous to the lysed bacteria, wherein the concentration of free NADH/NADPH in the cell-free protein synthesis system is less than 0.4 mM, wherein the cell-free protein synthesis system does not comprise tryptone, wherein the cell-free protein synthesis system does not comprise yeast extract, wherein the minimal media comprises at least one of (a) a carbon source; (b) a ammonium source; (c) a magnesium source; (d) a phosphate source; (e) a sulfur source; (f) a trace mineral source; and (g) sodium succinate, wherein the carbon source is chosen from D-glucose and glycerol, wherein the concentration of the D-glucose in the minimal media is from 0.1% to 2.5% (w/v), wherein the concentration of the glycerol in the minimal media is from 0.25% to 25% (w/v), wherein the concentration of the sodium succinate in the minimal media is from 0.05% to 5% (w/v), wherein the phosphate source comprises a sodium phosphate buffer or a potassium phosphate buffer, wherein the concentration of the potassium phosphate buffer in the minimal media is from 5 to 250 mM, wherein the pH of the potassium phosphate buffer in the minimal media is from 6.7 to 7.7, wherein the ammonium source comprises ammonium chloride, wherein the concentration of the ammonium chloride in the minimal media is from 5 to 250 mM, wherein the sulfur source comprises sodium sulfate, and wherein the sulfur source comprises sodium sulfate, wherein the concentration of the sodium sulfate in the minimal media is from 0.5 to 25 mM, wherein the magnesium source comprises magnesium sulfate heptahydrate, wherein the concentration of the magnesium sulfate heptahydrate in the minimal media is from 0.2 to 20 mM, wherein the trace mineral source is chosen from an iron source, a copper source, a nickel source, a zinc source, a molybdenum source, a boron source, and a manganese source, wherein the iron source comprises ferric citrate, wherein the concentration of the ferric citrate in the minimal media is from 10 to 1,000 μM, wherein the cell-free cell extract is prepared by preparing a suspension in which each one gram of the bacteria is suspended in 0.1 mL to 6.1 mL of a suspension buffer, wherein the cell-free cell extract is prepared by lysing the bacteria in the suspension, and wherein the lysing comprises sonication.

In some embodiments, the suspension buffer comprises one or more of (a) Tris-acetate; (b) magnesium glutamate; and (c) potassium glutamate. In some embodiments, the suspension buffer comprises (a) Tris-acetate; (b) magnesium glutamate; and (c) potassium glutamate. In some embodiments, provided herein is a cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the cell-free cell extract is prepared by preparing a suspension in which each one gram of the bacteria is suspended in 0.1 mL to 6.1 mL of a suspension buffer, and wherein the suspension buffer comprises one or more of (a) Tris-acetate; (b) magnesium glutamate; and (c) potassium glutamate.

In some embodiments, provided herein is a cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5, wherein the cell-free protein synthesis system is maintained at a temperature less than about 30° C., wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, wherein the molecular crowder is chosen from PEG 400-8000, maltodextrin, Ficoll 70-400, dextran, and serum albumin, optionally bovine serum albumin, wherein the concentration of the molecular crowder in the cell-free protein synthesis system is from about 0 to 40 g/L, wherein the carbon source, if present, is chosen from glucose, fructose, galactose, sucrose, lactose, gluconate, starch, maltodextrin, and maltose, wherein the concentration of the carbon source in the cell-free protein synthesis system is from about 0 to 50 g/L, wherein the ammonium source, if present, comprises ammonium sulfate, wherein the concentration of the ammonium source in the cell-free protein synthesis system is from 2 to 20 mM, wherein the potassium source, if present, is chosen from potassium glutamate and potassium gluconate, wherein the concentration of the potassium glutamate in the cell-free protein synthesis system is from about 10 to 270 mM, wherein the concentration of the potassium gluconate in the cell-free protein synthesis system is from about 10 to 250 mM, wherein the magnesium source, if present, comprises magnesium glutamate, wherein the concentration of the magnesium source in the cell-free protein synthesis system is from 0 to about 10 mM, wherein the phosphate source, if present, comprises potassium phosphate, wherein the concentration of the phosphate source in the cell-free protein synthesis system is from about 5 to about 23.5 mM, wherein the sulfur source, if present, comprises ammonium sulfate, wherein the concentration of the sulfur source in the cell-free protein synthesis system is from 2 to 20 mM, wherein the buffer comprises 3-(N-morpholino)propanesulfonic acid (MOPS) buffer, wherein the concentration of the buffer in the cell-free protein synthesis system is from 50 to 300 mM, wherein the concentration of the cell-free cell extract in the cell-free protein synthesis system is from 10% to 70% (v/v), wherein the concentration of the DNA or RNA encoding the protein of interest in the cell-free protein synthesis system is from 1 to 100 μg/mL, wherein the cell-free protein synthesis system comprises free amino acids endogenous to the lysed bacteria, wherein the concentration of free amino acids in the cell-free protein synthesis system is less than 2 mM, wherein the cell-free protein synthesis system comprises free nucleotides endogenous to the lysed bacteria, wherein the concentration of free nucleotides in the cell-free protein synthesis system is less than 1.5 mM, wherein the cell-free protein synthesis system comprises free NADH/NADPH endogenous to the lysed bacteria, wherein the concentration of free NADH/NADPH in the cell-free protein synthesis system is less than 0.4 mM, wherein the cell-free protein synthesis system does not comprise tryptone, wherein the cell-free protein synthesis system does not comprise yeast extract, wherein the minimal media comprises at least one of (a) a carbon source; (b) a ammonium source; (c) a magnesium source; (d) a phosphate source; (e) a sulfur source; (f) a trace mineral source; and (g) sodium succinate, wherein the carbon source is chosen from D-glucose and glycerol, wherein the concentration of the D-glucose in the minimal media is from 0.1% to 2.5% (w/v), wherein the concentration of the glycerol in the minimal media is from 0.25% to 25% (w/v), wherein the concentration of the sodium succinate in the minimal media is from 0.05% to 5% (w/v), wherein the phosphate source comprises a sodium phosphate buffer or a potassium phosphate buffer, wherein the concentration of the potassium phosphate buffer in the minimal media is from 5 to 250 mM, wherein the pH of the potassium phosphate buffer in the minimal media is from 6.7 to 7.7, wherein the ammonium source comprises ammonium chloride, wherein the concentration of the ammonium chloride in the minimal media is from 5 to 250 mM, wherein the sulfur source comprises sodium sulfate, and wherein the sulfur source comprises sodium sulfate, wherein the concentration of the sodium sulfate in the minimal media is from 0.5 to 25 mM, wherein the magnesium source comprises magnesium sulfate heptahydrate, wherein the concentration of the magnesium sulfate heptahydrate in the minimal media is from 0.2 to 20 mM, wherein the trace mineral source is chosen from an iron source, a copper source, a nickel source, a zinc source, a molybdenum source, a boron source, and a manganese source, wherein the iron source comprises ferric citrate, wherein the concentration of the ferric citrate in the minimal media is from 10 to 1,000 M, wherein the cell-free cell extract is prepared by preparing a suspension in which each one gram of the bacteria is suspended in 0.1 mL to 6.1 mL of a suspension buffer, wherein the cell-free cell extract is prepared by lysing the bacteria in the suspension, wherein the lysing comprises sonication, and wherein the suspension buffer comprises one or more of (a) Tris-acetate; (b) magnesium glutamate; and (c) potassium glutamate.

In some embodiments, the concentration of the Tris-acetate in the suspension buffer is from 1 to 100 mM. In some embodiments, the concentration of the Tris-acetate in the suspension buffer is from 2 to 90 mM. In some embodiments, the concentration of the Tris-acetate in the suspension buffer is from 3 to 80 mM. In some embodiments, the concentration of the Tris-acetate in the suspension buffer is from 4 to 70 mM. In some embodiments, the concentration of the Tris-acetate in the suspension buffer is from 5 to 60 mM. In some embodiments, the concentration of the Tris-acetate in the suspension buffer is from 6 to 50 mM. In some embodiments, the concentration of the Tris-acetate in the suspension buffer is from 7 to 40 mM. In some embodiments, the concentration of the Tris-acetate in the suspension buffer is from 8 to 30 mM. In some embodiments, the concentration of the Tris-acetate in the suspension buffer is from 9 to 20 mM. In some embodiments, the concentration of the Tris-acetate in the suspension buffer is from 9 to 15 mM. In some embodiments, the concentration of the Tris-acetate in the suspension buffer is from 9 to 12.5 mM. In some embodiments, the concentration of the Tris-acetate in the suspension buffer is from 9 to 11 mM. In some embodiments, the concentration of the Tris-acetate in the suspension buffer is 10 mM. In some embodiments, provided herein is a cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the cell-free cell extract is prepared by preparing a suspension in which each one gram of the bacteria is suspended in 0.1 mL to 6.1 mL of a suspension buffer, wherein the suspension buffer comprises one or more of (a) Tris-acetate; (b) magnesium glutamate; and (c) potassium glutamate, and wherein the concentration of the Tris-acetate in the suspension buffer is from 1 to 100 mM.

In some embodiments, provided herein is a cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5, wherein the cell-free protein synthesis system is maintained at a temperature less than about 30° C., wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, wherein the molecular crowder is chosen from PEG 400-8000, maltodextrin, Ficoll 70-400, dextran, and serum albumin, optionally bovine serum albumin, wherein the concentration of the molecular crowder in the cell-free protein synthesis system is from about 0 to 40 g/L, wherein the carbon source, if present, is chosen from glucose, fructose, galactose, sucrose, lactose, gluconate, starch, maltodextrin, and maltose, wherein the concentration of the carbon source in the cell-free protein synthesis system is from about 0 to 50 g/L, wherein the ammonium source, if present, comprises ammonium sulfate, wherein the concentration of the ammonium source in the cell-free protein synthesis system is from 2 to 20 mM, wherein the potassium source, if present, is chosen from potassium glutamate and potassium gluconate, wherein the concentration of the potassium glutamate in the cell-free protein synthesis system is from about 10 to 270 mM, wherein the concentration of the potassium gluconate in the cell-free protein synthesis system is from about 10 to 250 mM, wherein the magnesium source, if present, comprises magnesium glutamate, wherein the concentration of the magnesium source in the cell-free protein synthesis system is from 0 to about 10 mM, wherein the phosphate source, if present, comprises potassium phosphate, wherein the concentration of the phosphate source in the cell-free protein synthesis system is from about 5 to about 23.5 mM, wherein the sulfur source, if present, comprises ammonium sulfate, wherein the concentration of the sulfur source in the cell-free protein synthesis system is from 2 to 20 mM, wherein the buffer comprises 3-(N-morpholino)propanesulfonic acid (MOPS) buffer, wherein the concentration of the buffer in the cell-free protein synthesis system is from 50 to 300 mM, wherein the concentration of the cell-free cell extract in the cell-free protein synthesis system is from 10% to 70% (v/v), wherein the concentration of the DNA or RNA encoding the protein of interest in the cell-free protein synthesis system is from 1 to 100 μg/mL, wherein the cell-free protein synthesis system comprises free amino acids endogenous to the lysed bacteria, wherein the concentration of free amino acids in the cell-free protein synthesis system is less than 2 mM, wherein the cell-free protein synthesis system comprises free nucleotides endogenous to the lysed bacteria, wherein the concentration of free nucleotides in the cell-free protein synthesis system is less than 1.5 mM, wherein the cell-free protein synthesis system comprises free NADH/NADPH endogenous to the lysed bacteria, wherein the concentration of free NADH/NADPH in the cell-free protein synthesis system is less than 0.4 mM, wherein the cell-free protein synthesis system does not comprise tryptone, wherein the cell-free protein synthesis system does not comprise yeast extract, wherein the minimal media comprises at least one of (a) a carbon source; (b) a ammonium source; (c) a magnesium source; (d) a phosphate source; (e) a sulfur source; (f) a trace mineral source; and (g) sodium succinate, wherein the carbon source is chosen from D-glucose and glycerol, wherein the concentration of the D-glucose in the minimal media is from 0.1% to 2.5% (w/v), wherein the concentration of the glycerol in the minimal media is from 0.25% to 25% (w/v), wherein the concentration of the sodium succinate in the minimal media is from 0.05% to 5% (w/v), wherein the phosphate source comprises a sodium phosphate buffer or a potassium phosphate buffer, wherein the concentration of the potassium phosphate buffer in the minimal media is from 5 to 250 mM, wherein the pH of the potassium phosphate buffer in the minimal media is from 6.7 to 7.7, wherein the ammonium source comprises ammonium chloride, wherein the concentration of the ammonium chloride in the minimal media is from 5 to 250 mM, wherein the sulfur source comprises sodium sulfate, and wherein the sulfur source comprises sodium sulfate, wherein the concentration of the sodium sulfate in the minimal media is from 0.5 to 25 mM, wherein the magnesium source comprises magnesium sulfate heptahydrate, wherein the concentration of the magnesium sulfate heptahydrate in the minimal media is from 0.2 to 20 mM, wherein the trace mineral source is chosen from an iron source, a copper source, a nickel source, a zinc source, a molybdenum source, a boron source, and a manganese source, wherein the iron source comprises ferric citrate, wherein the concentration of the ferric citrate in the minimal media is from 10 to 1,000 M, wherein the cell-free cell extract is prepared by preparing a suspension in which each one gram of the bacteria is suspended in 0.1 mL to 6.1 mL of a suspension buffer, wherein the cell-free cell extract is prepared by lysing the bacteria in the suspension, wherein the lysing comprises sonication, wherein the suspension buffer comprises one or more of (a) Tris-acetate; (b) magnesium glutamate; and (c) potassium glutamate, and wherein the concentration of the Tris-acetate in the suspension buffer is from 1 to 100 mM.

In some embodiments, the concentration of the magnesium glutamate in the suspension buffer is from 1 to 100 mM. In some embodiments, the concentration of the magnesium glutamate in the suspension buffer is from 2 to 90 mM. In some embodiments, the concentration of the magnesium glutamate in the suspension buffer is from 3 to 80 mM. In some embodiments, the concentration of the magnesium glutamate in the suspension buffer is from 4 to 70 mM. In some embodiments, the concentration of the magnesium glutamate in the suspension buffer is from 5 to 60 mM. In some embodiments, the concentration of the magnesium glutamate in the suspension buffer is from 6 to 50 mM. In some embodiments, the concentration of the magnesium glutamate in the suspension buffer is from 7 to 40 mM. In some embodiments, the concentration of the magnesium glutamate in the suspension buffer is from 8 to 30 mM. In some embodiments, the concentration of the magnesium glutamate in the suspension buffer is from 9 to 20 mM. In some embodiments, the concentration of the magnesium glutamate in the suspension buffer is from 10 to 19 mM. In some embodiments, the concentration of the magnesium glutamate in the suspension buffer is from 11 to 18 mM. In some embodiments, the concentration of the magnesium glutamate in the suspension buffer is from 12 to 17 mM. In some embodiments, the concentration of the magnesium glutamate in the suspension buffer is from 13 to 16 mM. In some embodiments, the concentration of the magnesium glutamate in the suspension buffer is from 13 to 15 mM. In some embodiments, the concentration of the magnesium glutamate in the suspension buffer is 14 mM. In some embodiments, provided herein is a cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the cell-free cell extract is prepared by preparing a suspension in which each one gram of the bacteria is suspended in 0.1 mL to 6.1 mL of a suspension buffer, wherein the suspension buffer comprises one or more of (a) Tris-acetate; (b) magnesium glutamate; and (c) potassium glutamate, and wherein the concentration of the magnesium glutamate in the suspension buffer is from 1 to 100 mM.

In some embodiments, provided herein is a cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5, wherein the cell-free protein synthesis system is maintained at a temperature less than about 30° C., wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, wherein the molecular crowder is chosen from PEG 400-8000, maltodextrin, Ficoll 70-400, dextran, and serum albumin, optionally bovine serum albumin, wherein the concentration of the molecular crowder, if present, in the cell-free protein synthesis system is from about 0 to 40 g/L, wherein the carbon source is chosen from glucose, fructose, galactose, sucrose, lactose, gluconate, starch, maltodextrin, and maltose, wherein the concentration of the carbon source in the cell-free protein synthesis system is from about 0 to 50 g/L, wherein the ammonium source, if present, comprises ammonium sulfate, wherein the concentration of the ammonium source in the cell-free protein synthesis system is from 2 to 20 mM, wherein the potassium source, if present, is chosen from potassium glutamate and potassium gluconate, wherein the concentration of the potassium glutamate in the cell-free protein synthesis system is from about 10 to 270 mM, wherein the concentration of the potassium gluconate in the cell-free protein synthesis system is from about 10 to 250 mM, wherein the magnesium source, if present, comprises magnesium glutamate, wherein the concentration of the magnesium source in the cell-free protein synthesis system is from 0 to about 10 mM, wherein the phosphate source, if present, comprises potassium phosphate, wherein the concentration of the phosphate source in the cell-free protein synthesis system is from about 5 to about 23.5 mM, wherein the sulfur source, if present, comprises ammonium sulfate, wherein the concentration of the sulfur source in the cell-free protein synthesis system is from 2 to 20 mM, wherein the buffer comprises 3-(N-morpholino)propanesulfonic acid (MOPS) buffer, wherein the concentration of the buffer in the cell-free protein synthesis system is from 50 to 300 mM, wherein the concentration of the cell-free cell extract in the cell-free protein synthesis system is from 10% to 70% (v/v), wherein the concentration of the DNA or RNA encoding the protein of interest in the cell-free protein synthesis system is from 1 to 100 μg/mL, wherein the cell-free protein synthesis system comprises free amino acids endogenous to the lysed bacteria, wherein the concentration of free amino acids in the cell-free protein synthesis system is less than 2 mM, wherein the cell-free protein synthesis system comprises free nucleotides endogenous to the lysed bacteria, wherein the concentration of free nucleotides in the cell-free protein synthesis system is less than 1.5 mM, wherein the cell-free protein synthesis system comprises free NADH/NADPH endogenous to the lysed bacteria, wherein the concentration of free NADH/NADPH in the cell-free protein synthesis system is less than 0.4 mM, wherein the cell-free protein synthesis system does not comprise tryptone, wherein the cell-free protein synthesis system does not comprise yeast extract, wherein the minimal media comprises at least one of (a) a carbon source; (b) a ammonium source; (c) a magnesium source; (d) a phosphate source; (e) a sulfur source; (f) a trace mineral source; and (g) sodium succinate, wherein the carbon source is chosen from D-glucose and glycerol, wherein the concentration of the D-glucose in the minimal media is from 0.1% to 2.5% (w/v), wherein the concentration of the glycerol in the minimal media is from 0.25% to 25% (w/v), wherein the concentration of the sodium succinate in the minimal media is from 0.05% to 5% (w/v), wherein the phosphate source comprises a sodium phosphate buffer or a potassium phosphate buffer, wherein the concentration of the potassium phosphate buffer in the minimal media is from 5 to 250 mM, wherein the pH of the potassium phosphate buffer in the minimal media is from 6.7 to 7.7, wherein the ammonium source comprises ammonium chloride, wherein the concentration of the ammonium chloride in the minimal media is from 5 to 250 mM, wherein the sulfur source comprises sodium sulfate, and wherein the sulfur source comprises sodium sulfate, wherein the concentration of the sodium sulfate in the minimal media is from 0.5 to 25 mM, wherein the magnesium source comprises magnesium sulfate heptahydrate, wherein the concentration of the magnesium sulfate heptahydrate in the minimal media is from 0.2 to 20 mM, wherein the trace mineral source is chosen from an iron source, a copper source, a nickel source, a zinc source, a molybdenum source, a boron source, and a manganese source, wherein the iron source comprises ferric citrate, wherein the concentration of the ferric citrate in the minimal media is from 10 to 1,000 M, wherein the cell-free cell extract is prepared by preparing a suspension in which each one gram of the bacteria is suspended in 0.1 mL to 6.1 mL of a suspension buffer, wherein the cell-free cell extract is prepared by lysing the bacteria in the suspension, wherein the lysing comprises sonication, wherein the suspension buffer comprises one or more of (a) Tris-acetate; (b) magnesium glutamate; and (c) potassium glutamate, wherein the concentration of the Tris-acetate in the suspension buffer is from 1 to 100 mM, and wherein the concentration of the magnesium glutamate in the suspension buffer is from 1 to 100 mM.

In some embodiments, the concentration of the potassium glutamate in the suspension buffer is from 1 to 180 mM. In some embodiments, the concentration of the potassium glutamate in the suspension buffer is from 5 to 170 mM. In some embodiments, the concentration of the potassium glutamate in the suspension buffer is from 10 to 160 mM. In some embodiments, the concentration of the potassium glutamate in the suspension buffer is from 15 to 150 mM. In some embodiments, the concentration of the potassium glutamate in the suspension buffer is from 20 to 140 mM. In some embodiments, the concentration of the potassium glutamate in the suspension buffer is from 25 to 130 mM. In some embodiments, the concentration of the potassium glutamate in the suspension buffer is from 30 to 120 mM. In some embodiments, the concentration of the potassium glutamate in the suspension buffer is from 35 to 110 mM. In some embodiments, the concentration of the potassium glutamate in the suspension buffer is from 40 to 100 mM. In some embodiments, the concentration of the potassium glutamate in the suspension buffer is from 45 to 90 mM. In some embodiments, the concentration of the potassium glutamate in the suspension buffer is from 50 to 80 mM. In some embodiments, the concentration of the potassium glutamate in the suspension buffer is from 55 to 70 mM. In some embodiments, the concentration of the potassium glutamate in the suspension buffer is from 55 to 65 mM. In some embodiments, the concentration of the potassium glutamate in the suspension buffer is from 57.5 to 62.5 mM. In some embodiments, the concentration of the potassium glutamate in the suspension buffer is 60 mM. In some embodiments, provided herein is a cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the cell-free cell extract is prepared by preparing a suspension in which each one gram of the bacteria is suspended in 0.1 mL to 6.1 mL of a suspension buffer, wherein the suspension buffer comprises one or more of (a) Tris-acetate; (b) magnesium glutamate; and (c) potassium glutamate, and wherein the concentration of the potassium glutamate in the suspension buffer is from 1 to 180 mM.

In some embodiments, provided herein is a cell-free protein synthesis system comprising: (a) a cell-free cell extract comprising supernatant collected from lysed bacteria grown in minimal media; and (b) a reaction mixture comprising DNA or RNA encoding a protein of interest, wherein the cell-free protein synthesis system does not comprise free amino acids, free nucleotides, or free NADH/NADPH, except when endogenous to the lysed bacteria, wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5, wherein the cell-free protein synthesis system is maintained at a temperature less than about 30° C., wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, wherein the molecular crowder, if present, is chosen from PEG 400-8000, maltodextrin, Ficoll 70-400, dextran, and serum albumin, optionally bovine serum albumin, wherein the concentration of the molecular crowder in the cell-free protein synthesis system is from about 0 to 40 g/L, wherein the carbon source, if present, is chosen from glucose, fructose, galactose, sucrose, lactose, gluconate, starch, maltodextrin, and maltose, wherein the concentration of the carbon source in the cell-free protein synthesis system is from about 0 to 50 g/L, wherein the ammonium source, if present, comprises ammonium sulfate, wherein the concentration of the ammonium source in the cell-free protein synthesis system is from 2 to 20 mM, wherein the potassium source, if present, is chosen from potassium glutamate and potassium gluconate, wherein the concentration of the potassium glutamate in the cell-free protein synthesis system is from about 10 to 270 mM, wherein the concentration of the potassium gluconate in the cell-free protein synthesis system is from about 10 to 250 mM, wherein the magnesium source, if present, comprises magnesium glutamate, wherein the concentration of the magnesium source in the cell-free protein synthesis system is from 0 to about 10 mM, wherein the phosphate source, if present, comprises potassium phosphate, wherein the concentration of the phosphate source in the cell-free protein synthesis system is from about 5 to about 23.5 mM, wherein the sulfur source, if present, comprises ammonium sulfate, wherein the concentration of the sulfur source in the cell-free protein synthesis system is from 2 to 20 mM, wherein the buffer comprises 3-(N-morpholino)propanesulfonic acid (MOPS) buffer, wherein the concentration of the buffer in the cell-free protein synthesis system is from 50 to 300 mM, wherein the concentration of the cell-free cell extract in the cell-free protein synthesis system is from 10% to 70% (v/v), wherein the concentration of the DNA or RNA encoding the protein of interest in the cell-free protein synthesis system is from 1 to 100 μg/mL, wherein the cell-free protein synthesis system comprises free amino acids endogenous to the lysed bacteria, wherein the concentration of free amino acids in the cell-free protein synthesis system is less than 2 mM, wherein the cell-free protein synthesis system comprises free nucleotides endogenous to the lysed bacteria, wherein the concentration of free nucleotides in the cell-free protein synthesis system is less than 1.5 mM, wherein the cell-free protein synthesis system comprises free NADH/NADPH endogenous to the lysed bacteria, wherein the concentration of free NADH/NADPH in the cell-free protein synthesis system is less than 0.4 mM, wherein the cell-free protein synthesis system does not comprise tryptone, wherein the cell-free protein synthesis system does not comprise yeast extract, wherein the minimal media comprises at least one of (a) a carbon source; (b) a ammonium source; (c) a magnesium source; (d) a phosphate source; (e) a sulfur source; (f) a trace mineral source; and (g) sodium succinate, wherein the carbon source is chosen from D-glucose and glycerol, wherein the concentration of the D-glucose in the minimal media is from 0.1% to 2.5% (w/v), wherein the concentration of the glycerol in the minimal media is from 0.25% to 25% (w/v), wherein the concentration of the sodium succinate in the minimal media is from 0.05% to 5% (w/v), wherein the phosphate source comprises a sodium phosphate buffer or a potassium phosphate buffer, wherein the concentration of the potassium phosphate buffer in the minimal media is from 5 to 250 mM, wherein the pH of the potassium phosphate buffer in the minimal media is from 6.7 to 7.7, wherein the ammonium source comprises ammonium chloride, wherein the concentration of the ammonium chloride in the minimal media is from 5 to 250 mM, wherein the sulfur source comprises sodium sulfate, and wherein the sulfur source comprises sodium sulfate, wherein the concentration of the sodium sulfate in the minimal media is from 0.5 to 25 mM, wherein the magnesium source comprises magnesium sulfate heptahydrate, wherein the concentration of the magnesium sulfate heptahydrate in the minimal media is from 0.2 to 20 mM, wherein the trace mineral source is chosen from an iron source, a copper source, a nickel source, a zinc source, a molybdenum source, a boron source, and a manganese source, wherein the iron source comprises ferric citrate, wherein the concentration of the ferric citrate in the minimal media is from 10 to 1,000 M, wherein the cell-free cell extract is prepared by preparing a suspension in which each one gram of the bacteria is suspended in 0.1 mL to 6.1 mL of a suspension buffer, wherein the cell-free cell extract is prepared by lysing the bacteria in the suspension, wherein the lysing comprises sonication, wherein the suspension buffer comprises one or more of (a) Tris-acetate; (b) magnesium glutamate; and (c) potassium glutamate, wherein the concentration of the Tris-acetate in the suspension buffer is from 1 to 100 mM, wherein the concentration of the magnesium glutamate in the suspension buffer is from 1 to 100 mM, and wherein the concentration of the potassium glutamate in the suspension buffer is from 1 to 180 mM.

### Cell-Free Protein Synthesis

In some embodiments, provided herein is a method for preparing a protein of interest, comprising using the cell-free protein synthesis system of any one of the embodiments disclosed herein. In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the system produces the protein of interest when maintained at the temperature. In some embodiments, the method comprises (3) purifying the protein of interest.

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the system produces the protein of interest when maintained at the temperature, and wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5.

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the system produces the protein of interest when maintained at the temperature, and wherein the temperature is less than about 30° C.

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the system produces the protein of interest when maintained at the temperature, wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5, and wherein the temperature is less than about 30° C.

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the system produces the protein of interest when maintained at the temperature, and wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer.

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5, wherein the temperature is less than about 30° C., and wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer.

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the system produces the protein of interest when maintained at the temperature, wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, and wherein the molecular crowder is chosen from PEG 400-8000, maltodextrin, Ficoll 70-400, dextran, and serum albumin, optionally bovine serum albumin.

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the system produces the protein of interest when maintained at the temperature, wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5, wherein the temperature is less than about 30° C., wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, and wherein the molecular crowder is chosen from PEG 400-8000, maltodextrin, Ficoll 70-400, dextran, and serum albumin, optionally bovine serum albumin.

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, and wherein the concentration of the molecular crowder in the cell-free protein synthesis system is from about 0 to 40 g/L.

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the system produces the protein of interest when maintained at the temperature, wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5, wherein the temperature is less than about 30° C., wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, wherein the molecular crowder is chosen from PEG 400-8000, maltodextrin, Ficoll 70-400, dextran, and serum albumin, optionally bovine serum albumin, and wherein the concentration of the molecular crowder in the cell-free protein synthesis system is from about 0 to 40 g/L.

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the system produces the protein of interest when maintained at the temperature, wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, and wherein the carbon source is chosen from glucose, fructose, galactose, sucrose, lactose, gluconate, starch, maltodextrin, and maltose.

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the system produces the protein of interest when maintained at the temperature, wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5, wherein the temperature is less than about 30° C., wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, wherein the molecular crowder is chosen from PEG 400-8000, maltodextrin, Ficoll 70-400, dextran, and serum albumin, optionally bovine serum albumin, wherein the concentration of the molecular crowder in the cell-free protein synthesis system is from about 0 to 40 g/L, and wherein the carbon source is chosen from glucose, fructose, galactose, sucrose, lactose, gluconate, starch, maltodextrin, and maltose.

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the system produces the protein of interest when maintained at the temperature, wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, and wherein the concentration of the carbon source in the cell-free protein synthesis system is from about 0 to 50 g/L.

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5, wherein the temperature is less than about 30° C., wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, wherein the molecular crowder is chosen from PEG 400-8000, maltodextrin, Ficoll 70-400, dextran, and serum albumin, optionally bovine serum albumin, wherein the concentration of the molecular crowder in the cell-free protein synthesis system is from about 0 to 40 g/L, wherein the carbon source is chosen from glucose, fructose, galactose, sucrose, lactose, gluconate, starch, maltodextrin, and maltose, and wherein the concentration of the carbon source in the cell-free protein synthesis system is from about 0 to 50 g/L.

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the system produces the protein of interest when maintained at the temperature, wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, and wherein the concentration of the ammonium source in the cell-free protein synthesis system is from 2 to 20 mM.

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the system produces the protein of interest when maintained at the temperature, wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5, wherein the temperature is less than about 30° C., wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, wherein the molecular crowder is chosen from PEG 400-8000, maltodextrin, Ficoll 70-400, dextran, and serum albumin, optionally bovine serum albumin, wherein the concentration of the molecular crowder, if present, in the cell-free protein synthesis system is from about 0 to 40 g/L, wherein the carbon source, if present, is chosen from glucose, fructose, galactose, sucrose, lactose, gluconate, starch, maltodextrin, and maltose, wherein the concentration of the carbon source in the cell-free protein synthesis system is from about 0 to 50 g/L, and wherein the concentration of the ammonium source, if present, in the cell-free protein synthesis system is from 2 to 20 mM.

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the system produces the protein of interest when maintained at the temperature, wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, and wherein the potassium source is chosen from potassium glutamate and potassium gluconate.

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the system produces the protein of interest when maintained at the temperature, wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5, wherein the temperature is less than about 30° C., wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, wherein the molecular crowder, if present, is chosen from PEG 400-8000, maltodextrin, Ficoll 70-400, dextran, and serum albumin, optionally bovine serum albumin, wherein the concentration of the molecular crowder, if present, in the cell-free protein synthesis system is from about 0 to 40 g/L, wherein the carbon source, if present, is chosen from glucose, fructose, galactose, sucrose, lactose, gluconate, starch, maltodextrin, and maltose, wherein the concentration of the carbon source in the cell-free protein synthesis system is from about 0 to 50 g/L, wherein the concentration of the ammonium source, if present, in the cell-free protein synthesis system is from 2 to 20 mM, and wherein the potassium source is chosen from potassium glutamate and potassium gluconate.

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the system produces the protein of interest when maintained at the temperature, wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, and wherein the concentration of the potassium glutamate in the cell-free protein synthesis system is from about 10 to 270 mM.

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the system produces the protein of interest when maintained at the temperature, wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5, wherein the temperature is less than about 30° C., wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, wherein the molecular crowder is chosen from PEG 400-8000, maltodextrin, Ficoll 70-400, dextran, and serum albumin, optionally bovine serum albumin, wherein the concentration of the molecular crowder in the cell-free protein synthesis system is from about 0 to 40 g/L, wherein the carbon source, if present, is chosen from glucose, fructose, galactose, sucrose, lactose, gluconate, starch, maltodextrin, and maltose, wherein the concentration of the carbon source in the cell-free protein synthesis system is from about 0 to 50 g/L, wherein the concentration of the ammonium source, if present, in the cell-free protein synthesis system is from 2 to 20 mM, wherein the potassium source is chosen from potassium glutamate and potassium gluconate, and wherein the concentration of the potassium glutamate in the cell-free protein synthesis system is from about 10 to 270 mM.

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the system produces the protein of interest when maintained at the temperature, wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, and wherein the concentration of the potassium gluconate in the cell-free protein synthesis system is from about 10 to 250 mM.

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5, wherein the temperature is less than about 30° C., wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, wherein the molecular crowder, if present, is chosen from PEG 400-8000, maltodextrin, Ficoll 70-400, dextran, and serum albumin, optionally bovine serum albumin, wherein the concentration of the molecular crowder in the cell-free protein synthesis system is from about 0 to 40 g/L, wherein the carbon source, if present, is chosen from glucose, fructose, galactose, sucrose, lactose, gluconate, starch, maltodextrin, and maltose, wherein the concentration of the carbon source in the cell-free protein synthesis system is from about 0 to 50 g/L, wherein the concentration of the ammonium source, if present, in the cell-free protein synthesis system is from 2 to 20 mM, wherein the potassium source, if present, is chosen from potassium glutamate and potassium gluconate, wherein the concentration of the potassium glutamate in the cell-free protein synthesis system is from about 10 to 270 mM, and wherein the concentration of the potassium gluconate in the cell-free protein synthesis system is from about 10 to 250 mM.

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the system produces the protein of interest when maintained at the temperature, wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, and wherein the concentration of the magnesium source in the cell-free protein synthesis system is from 0 to about 10 mM.

In some embodiments, provided herein is a method for preparing a protein of interest, comprising ((1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the system produces the protein of interest when maintained at the temperature, wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5, wherein the temperature is less than about 30° C., wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, wherein the molecular crowder, if present, is chosen from PEG 400-8000, maltodextrin, Ficoll 70-400, dextran, and serum albumin, optionally bovine serum albumin, wherein the concentration of the molecular crowder in the cell-free protein synthesis system is from about 0 to 40 g/L, wherein the carbon source is chosen from glucose, fructose, galactose, sucrose, lactose, gluconate, starch, maltodextrin, and maltose, wherein the concentration of the carbon source in the cell-free protein synthesis system is from about 0 to 50 g/L, wherein the concentration of the ammonium source, if present, in the cell-free protein synthesis system is from 2 to 20 mM, wherein the potassium source, if present, is chosen from potassium glutamate and potassium gluconate, wherein the concentration of the potassium glutamate in the cell-free protein synthesis system is from about 10 to 270 mM, wherein the concentration of the potassium gluconate in the cell-free protein synthesis system is from about 10 to 250 mM, and wherein the concentration of the magnesium source, if present, in the cell-free protein synthesis system is from 0 to about 10 mM.

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the system produces the protein of interest when maintained at the temperature, wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, and wherein the concentration of the phosphate source in the cell-free protein synthesis system is from about 5 to about 23.5 mM.

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5, wherein the temperature is less than about 30° C., wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, wherein the molecular crowder is chosen from PEG 400-8000, maltodextrin, Ficoll 70-400, dextran, and serum albumin, optionally bovine serum albumin, wherein the concentration of the molecular crowder, if present, in the cell-free protein synthesis system is from about 0 to 40 g/L, wherein the carbon source, if present, is chosen from glucose, fructose, galactose, sucrose, lactose, gluconate, starch, maltodextrin, and maltose, wherein the concentration of the carbon source in the cell-free protein synthesis system is from about 0 to 50 g/L, wherein the concentration of the ammonium source, if present, in the cell-free protein synthesis system is from 2 to 20 mM, wherein the potassium source, if present, is chosen from potassium glutamate and potassium gluconate, wherein the concentration of the potassium glutamate in the cell-free protein synthesis system is from about 10 to 270 mM, wherein the concentration of the potassium gluconate in the cell-free protein synthesis system is from about 10 to 250 mM, wherein the concentration of the magnesium source, if present, in the cell-free protein synthesis system is from 0 to about 10 mM, and wherein the concentration of the phosphate source, if present, in the cell-free protein synthesis system is from about 5 to about 23.5 mM.

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the system produces the protein of interest when maintained at the temperature, wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, and wherein the concentration of the sulfur source in the cell-free protein synthesis system is from 2 to 20 mM.

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the system produces the protein of interest when maintained at the temperature, wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5, wherein the temperature is less than about 30° C., wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, wherein the molecular crowder, if present, is chosen from PEG 400-8000, maltodextrin, Ficoll 70-400, dextran, and serum albumin, optionally bovine serum albumin, wherein the concentration of the molecular crowder in the cell-free protein synthesis system is from about 0 to 40 g/L, wherein the carbon source, if present, is chosen from glucose, fructose, galactose, sucrose, lactose, gluconate, starch, maltodextrin, and maltose, wherein the concentration of the carbon source in the cell-free protein synthesis system is from about 0 to 50 g/L, wherein the concentration of the ammonium source, if present, in the cell-free protein synthesis system is from 2 to 20 mM, wherein the potassium source, if present, is chosen from potassium glutamate and potassium gluconate, wherein the concentration of the potassium glutamate in the cell-free protein synthesis system is from about 10 to 270 mM, wherein the concentration of the potassium gluconate in the cell-free protein synthesis system is from about 10 to 250 mM, wherein the concentration of the magnesium source, if present, in the cell-free protein synthesis system is from 0 to about 10 mM, wherein the concentration of the phosphate source, if present, in the cell-free protein synthesis system is from about 5 to about 23.5 mM, and wherein the concentration of the sulfur source, if present, in the cell-free protein synthesis system is from 2 to 20 mM.

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the system produces the protein of interest when maintained at the temperature, wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, and wherein the concentration of the buffer in the cell-free protein synthesis system is from 50 to 300 mM.

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5, wherein the temperature is less than about 30° C., wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, wherein the molecular crowder is chosen from PEG 400-8000, maltodextrin, Ficoll 70-400, dextran, and serum albumin, optionally bovine serum albumin, wherein the concentration of the molecular crowder, if present, in the cell-free protein synthesis system is from about 0 to 40 g/L, wherein the carbon source, if present, is chosen from glucose, fructose, galactose, sucrose, lactose, gluconate, starch, maltodextrin, and maltose, wherein the concentration of the carbon source in the cell-free protein synthesis system is from about 0 to 50 g/L, wherein the concentration of the ammonium source, if present, in the cell-free protein synthesis system is from 2 to 20 mM, wherein the potassium source, if present, is chosen from potassium glutamate and potassium gluconate, wherein the concentration of the potassium glutamate in the cell-free protein synthesis system is from about 10 to 270 mM, wherein the concentration of the potassium gluconate in the cell-free protein synthesis system is from about 10 to 250 mM, wherein the concentration of the magnesium source, if present, in the cell-free protein synthesis system is from 0 to about 10 mM, wherein the concentration of the phosphate source, if present, in the cell-free protein synthesis system is from about 5 to about 23.5 mM, wherein the concentration of the sulfur source, if present, in the cell-free protein synthesis system is from 2 to 20 mM, and wherein the concentration of the buffer in the cell-free protein synthesis system is from 50 to 300 mM.

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the system produces the protein of interest when maintained at the temperature, and wherein the concentration of the cell-free cell extract in the cell-free protein synthesis system is from 10% to 70% (v/v).

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the system produces the protein of interest when maintained at the temperature, wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5, wherein the temperature is less than about 30° C., wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, wherein the molecular crowder, if present, is chosen from PEG 400-8000, maltodextrin, Ficoll 70-400, dextran, and serum albumin, optionally bovine serum albumin, wherein the concentration of the molecular crowder in the cell-free protein synthesis system is from about 0 to 40 g/L, wherein the carbon source, if present, is chosen from glucose, fructose, galactose, sucrose, lactose, gluconate, starch, maltodextrin, and maltose, wherein the concentration of the carbon source in the cell-free protein synthesis system is from about 0 to 50 g/L, wherein the concentration of the ammonium source, if present, in the cell-free protein synthesis system is from 2 to 20 mM, wherein the potassium source, if present, is chosen from potassium glutamate and potassium gluconate, wherein the concentration of the potassium glutamate in the cell-free protein synthesis system is from about 10 to 270 mM, wherein the concentration of the potassium gluconate in the cell-free protein synthesis system is from about 10 to 250 mM, wherein the concentration of the magnesium source, if present, in the cell-free protein synthesis system is from 0 to about 10 mM, wherein the concentration of the phosphate source, if present, in the cell-free protein synthesis system is from about 5 to about 23.5 mM, wherein the concentration of the sulfur source, if present, in the cell-free protein synthesis system is from 2 to 20 mM, and wherein the concentration of the buffer in the cell-free protein synthesis system is from 50 to 300 mM, and wherein the concentration of the cell-free cell extract in the cell-free protein synthesis system is from 10% to 70% (v/v).

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the system produces the protein of interest when maintained at the temperature, and wherein the concentration of the DNA or RNA encoding the protein of interest in the cell-free protein synthesis system is from 1 to 100 μg/mL.

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the system produces the protein of interest when maintained at the temperature, wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5, wherein the temperature is less than about 30° C., wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, wherein the molecular crowder, if present, is chosen from PEG 400-8000, maltodextrin, Ficoll 70-400, dextran, and serum albumin, optionally bovine serum albumin, wherein the concentration of the molecular crowder in the cell-free protein synthesis system is from about 0 to 40 g/L, wherein the carbon source, if present, is chosen from glucose, fructose, galactose, sucrose, lactose, gluconate, starch, maltodextrin, and maltose, wherein the concentration of the carbon source, if present, in the cell-free protein synthesis system is from about 0 to 50 g/L, wherein the concentration of the ammonium source, if present, in the cell-free protein synthesis system is from 2 to 20 mM, wherein the potassium source, if present, is chosen from potassium glutamate and potassium gluconate, wherein the concentration of the potassium glutamate in the cell-free protein synthesis system is from about 10 to 270 mM, wherein the concentration of the potassium gluconate in the cell-free protein synthesis system is from about 10 to 250 mM, wherein the concentration of the magnesium source, if present, in the cell-free protein synthesis system is from 0 to about 10 mM, wherein the concentration of the phosphate source, if present, in the cell-free protein synthesis system is from about 5 to about 23.5 mM, wherein the concentration of the sulfur source, if present, in the cell-free protein synthesis system is from 2 to 20 mM, and wherein the concentration of the buffer in the cell-free protein synthesis system is from 50 to 300 mM, wherein the concentration of the cell-free cell extract in the cell-free protein synthesis system is from 10% to 70% (v/v), and wherein the concentration of the DNA or RNA encoding the protein of interest in the cell-free protein synthesis system is from 1 to 100 μg/mL.

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the system produces the protein of interest when maintained at the temperature, and wherein the concentration of free amino acids in the cell-free protein synthesis system is less than 2 mM.

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the system produces the protein of interest when maintained at the temperature, wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5, wherein the temperature is less than about 30° C., wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, wherein the molecular crowder, if present, is chosen from PEG 400-8000, maltodextrin, Ficoll 70-400, dextran, and serum albumin, optionally bovine serum albumin, wherein the concentration of the molecular crowder in the cell-free protein synthesis system is from about 0 to 40 g/L, wherein the carbon source, if present, is chosen from glucose, fructose, galactose, sucrose, lactose, gluconate, starch, maltodextrin, and maltose, wherein the concentration of the carbon source in the cell-free protein synthesis system is from about 0 to 50 g/L, wherein the concentration of the ammonium source, if present, in the cell-free protein synthesis system is from 2 to 20 mM, wherein the potassium source, if present, is chosen from potassium glutamate and potassium gluconate, wherein the concentration of the potassium glutamate in the cell-free protein synthesis system is from about 10 to 270 mM, wherein the concentration of the potassium gluconate in the cell-free protein synthesis system is from about 10 to 250 mM, wherein the concentration of the magnesium source, if present, in the cell-free protein synthesis system is from 0 to about 10 mM, wherein the concentration of the phosphate source, if present, in the cell-free protein synthesis system is from about 5 to about 23.5 mM, wherein the concentration of the sulfur source, if present, in the cell-free protein synthesis system is from 2 to 20 mM, and wherein the concentration of the buffer in the cell-free protein synthesis system is from 50 to 300 mM, wherein the concentration of the cell-free cell extract in the cell-free protein synthesis system is from 10% to 70% (v/v), wherein the concentration of the DNA or RNA encoding the protein of interest in the cell-free protein synthesis system is from 1 to 100 μg/mL, and wherein the concentration of free amino acids in the cell-free protein synthesis system is less than 2 mM.

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the system does not comprise free amino acids, free nucleotides, or free NADH/NADPH, except when endogenous to the lysed bacteria; and (2) maintaining the system at a temperature, wherein the system produces the protein of interest when maintained at the temperature, and wherein the concentration of free nucleotides in the cell-free protein synthesis system is less than 1.5 mM.

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the system produces the protein of interest when maintained at the temperature, wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5, wherein the temperature is less than about 30° C., wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, wherein the molecular crowder, if present, is chosen from PEG 400-8000, maltodextrin, Ficoll 70-400, dextran, and serum albumin, optionally bovine serum albumin, wherein the concentration of the molecular crowder in the cell-free protein synthesis system is from about 0 to 40 g/L, wherein the carbon source, if present, is chosen from glucose, fructose, galactose, sucrose, lactose, gluconate, starch, maltodextrin, and maltose, wherein the concentration of the carbon source in the cell-free protein synthesis system is from about 0 to 50 g/L, wherein the concentration of the ammonium source, if present, in the cell-free protein synthesis system is from 2 to 20 mM, wherein the potassium source, if present, is chosen from potassium glutamate and potassium gluconate, wherein the concentration of the potassium glutamate in the cell-free protein synthesis system is from about 10 to 270 mM, wherein the concentration of the potassium gluconate in the cell-free protein synthesis system is from about 10 to 250 mM, wherein the concentration of the magnesium source, if present, in the cell-free protein synthesis system is from 0 to about 10 mM, wherein the concentration of the phosphate source, if present, in the cell-free protein synthesis system is from about 5 to about 23.5 mM, wherein the concentration of the sulfur source, if present, in the cell-free protein synthesis system is from 2 to 20 mM, and wherein the concentration of the buffer in the cell-free protein synthesis system is from 50 to 300 mM, wherein the concentration of the cell-free cell extract in the cell-free protein synthesis system is from 10% to 70% (v/v), wherein the concentration of the DNA or RNA encoding the protein of interest in the cell-free protein synthesis system is from 1 to 100 μg/mL, wherein the concentration of free amino acids in the cell-free protein synthesis system is less than 2 mM, and wherein the concentration of free nucleotides in the cell-free protein synthesis system is less than 1.5 mM.

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the system produces the protein of interest when maintained at the temperature, and wherein the concentration of free NADH/NADPH in the cell-free protein synthesis system is less than 0.4 mM.

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the system produces the protein of interest when maintained at the temperature, wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5, wherein the temperature is less than about 30° C., wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, wherein the molecular crowder, if present, is chosen from PEG 400-8000, maltodextrin, Ficoll 70-400, dextran, and serum albumin, optionally bovine serum albumin, wherein the concentration of the molecular crowder in the cell-free protein synthesis system is from about 0 to 40 g/L, wherein the carbon source, if present, is chosen from glucose, fructose, galactose, sucrose, lactose, gluconate, starch, maltodextrin, and maltose, wherein the concentration of the carbon source, if present, in the cell-free protein synthesis system is from about 0 to 50 g/L, wherein the concentration of the ammonium source, if present, in the cell-free protein synthesis system is from 2 to 20 mM, wherein the potassium source, if present, is chosen from potassium glutamate and potassium gluconate, wherein the concentration of the potassium glutamate in the cell-free protein synthesis system is from about 10 to 270 mM, wherein the concentration of the potassium gluconate in the cell-free protein synthesis system is from about 10 to 250 mM, wherein the concentration of the magnesium source, if present, in the cell-free protein synthesis system is from 0 to about 10 mM, wherein the concentration of the phosphate source, if present, in the cell-free protein synthesis system is from about 5 to about 23.5 mM, wherein the concentration of the sulfur source, if present, in the cell-free protein synthesis system is from 2 to 20 mM, and wherein the concentration of the buffer in the cell-free protein synthesis system is from 50 to 300 mM, wherein the concentration of the cell-free cell extract in the cell-free protein synthesis system is from 10% to 70% (v/v), wherein the concentration of the DNA or RNA encoding the protein of interest in the cell-free protein synthesis system is from 1 to 100 μg/mL, wherein the concentration of free amino acids in the cell-free protein synthesis system is less than 2 mM, and wherein the concentration of free NADH/NADPH in the cell-free protein synthesis system is less than 0.4 mM.

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the system produces the protein of interest when maintained at the temperature, and wherein the cell-free protein synthesis system does not comprise tryptone. In some embodiments, provided herein is a method for preparing a protein of interest, comprising (1) obtaining a cell-free protein synthesis system, the system comprising: (a) a cell-free cell extract comprising supernatant collected from lysed bacteria grown in minimal media; and (b) a reaction mixture comprising DNA or RNA encoding a protein of interest, wherein the system does not comprise free amino acids, free nucleotides, or free NADH/NADPH, except when endogenous to the lysed bacteria; and (2) maintaining the system at a temperature, wherein the system produces the protein of interest when maintained at the temperature, and wherein the cell-free protein synthesis system does not comprise yeast extract.

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the system produces the protein of interest when maintained at the temperature, wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5, wherein the temperature is less than about 30° C., wherein the reaction mixture further comprises at least one of (a) a molecular crowder; (b) a carbon source; (c) a ammonium source; (d) a potassium source; (e) a magnesium source; (f) a phosphate source; and (g) a sulfur source; and (h) a buffer, wherein the molecular crowder, if present, is chosen from PEG 400-8000, maltodextrin, Ficoll 70-400, dextran, and serum albumin, optionally bovine serum albumin, wherein the concentration of the molecular crowder in the cell-free protein synthesis system is from about 0 to 40 g/L, wherein the carbon source, if present, is chosen from glucose, fructose, galactose, sucrose, lactose, gluconate, starch, maltodextrin, and maltose, wherein the concentration of the carbon source in the cell-free protein synthesis system is from about 0 to 50 g/L, wherein the concentration of the ammonium source, if present, in the cell-free protein synthesis system is from 2 to 20 mM, wherein the potassium source, if present, is chosen from potassium glutamate and potassium gluconate, wherein the concentration of the potassium glutamate in the cell-free protein synthesis system is from about 10 to 270 mM, wherein the concentration of the potassium gluconate in the cell-free protein synthesis system is from about 10 to 250 mM, wherein the concentration of the magnesium source, if present, in the cell-free protein synthesis system is from 0 to about 10 mM, wherein the concentration of the phosphate source, if present, in the cell-free protein synthesis system is from about 5 to about 23.5 mM, wherein the concentration of the sulfur source, if present, in the cell-free protein synthesis system is from 2 to 20 mM, and wherein the concentration of the buffer in the cell-free protein synthesis system is from 50 to 300 mM, wherein the concentration of the cell-free cell extract in the cell-free protein synthesis system is from 10% to 70% (v/v), wherein the concentration of the DNA or RNA encoding the protein of interest in the cell-free protein synthesis system is from 1 to 100 μg/mL, wherein the concentration of free amino acids in the cell-free protein synthesis system is less than 2 mM, wherein the concentration of free NADH/NADPH in the cell-free protein synthesis system is less than 0.4 mM, wherein the cell-free protein synthesis system does not comprise tryptone, and wherein the cell-free protein synthesis system does not comprise yeast extract.

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the system produces the protein of interest when maintained at the temperature, and wherein the minimal media comprises at least one of (a) a carbon source; (b) a ammonium source; (c) a magnesium source; (d) a phosphate source; (e) a sulfur source; (f) a trace mineral source; and (g) sodium succinate.

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the system produces the protein of interest when maintained at the temperature, wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5, wherein the temperature is less than about 30° C., wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, wherein the molecular crowder, if present, is chosen from PEG 400-8000, maltodextrin, Ficoll 70-400, dextran, and serum albumin, optionally bovine serum albumin, wherein the concentration of the molecular crowder in the cell-free protein synthesis system is from about 0 to 40 g/L, wherein the carbon source, if present, is chosen from glucose, fructose, galactose, sucrose, lactose, gluconate, starch, maltodextrin, and maltose, wherein the concentration of the carbon source in the cell-free protein synthesis system is from about 0 to 50 g/L, wherein the concentration of the ammonium source, if present, in the cell-free protein synthesis system is from 2 to 20 mM, wherein the potassium source, if present, is chosen from potassium glutamate and potassium gluconate, wherein the concentration of the potassium glutamate in the cell-free protein synthesis system is from about 10 to 270 mM, wherein the concentration of the potassium gluconate in the cell-free protein synthesis system is from about 10 to 250 mM, wherein the concentration of the magnesium source, if present, in the cell-free protein synthesis system is from 0 to about 10 mM, wherein the concentration of the phosphate source, if present, in the cell-free protein synthesis system is from about 5 to about 23.5 mM, wherein the concentration of the sulfur source, if present, in the cell-free protein synthesis system is from 2 to 20 mM, and wherein the concentration of the buffer in the cell-free protein synthesis system is from 50 to 300 mM, wherein the concentration of the cell-free cell extract in the cell-free protein synthesis system is from 10% to 70% (v/v), wherein the concentration of the DNA or RNA encoding the protein of interest in the cell-free protein synthesis system is from 1 to 100 μg/mL, wherein the concentration of free amino acids in the cell-free protein synthesis system is less than 2 mM, wherein the concentration of free NADH/NADPH in the cell-free protein synthesis system is less than 0.4 mM, wherein the cell-free protein synthesis system does not comprise tryptone, wherein the cell-free protein synthesis system does not comprise yeast extract, and wherein the minimal media comprises at least one of (a) a carbon source; (b) a ammonium source; (c) a magnesium source; (d) a phosphate source; (e) a sulfur source; (f) a trace mineral source; and (g) sodium succinate.

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the system produces the protein of interest when maintained at the temperature, wherein the minimal media comprises at least one of (a) a carbon source; (b) a ammonium source; (c) a magnesium source; (d) a phosphate source; (e) a sulfur source; (f) a trace mineral source; and (g) sodium succinate, wherein the carbon source is chosen from D-glucose and glycerol, and wherein the concentration of the D-glucose in the minimal media is from 0.1% to 2.5% (w/v).

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the system produces the protein of interest when maintained at the temperature, wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5, wherein the temperature is less than about 30° C., wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, wherein the molecular crowder, if present, is chosen from PEG 400-8000, maltodextrin, Ficoll 70-400, dextran, and serum albumin, optionally bovine serum albumin, wherein the concentration of the molecular crowder in the cell-free protein synthesis system is from about 0 to 40 g/L, wherein the carbon source, if present, is chosen from glucose, fructose, galactose, sucrose, lactose, gluconate, starch, maltodextrin, and maltose, wherein the concentration of the carbon source, if present, in the cell-free protein synthesis system is from about 0 to 50 g/L, wherein the concentration of the ammonium source, if present, in the cell-free protein synthesis system is from 2 to 20 mM, wherein the potassium source, if present, is chosen from potassium glutamate and potassium gluconate, wherein the concentration of the potassium glutamate in the cell-free protein synthesis system is from about 10 to 270 mM, wherein the concentration of the potassium gluconate in the cell-free protein synthesis system is from about 10 to 250 mM, wherein the concentration of the magnesium source, if present, in the cell-free protein synthesis system is from 0 to about 10 mM, wherein the concentration of the phosphate source, if present, in the cell-free protein synthesis system is from about 5 to about 23.5 mM, wherein the concentration of the sulfur source, if present, in the cell-free protein synthesis system is from 2 to 20 mM, and wherein the concentration of the buffer in the cell-free protein synthesis system is from 50 to 300 mM, wherein the concentration of the cell-free cell extract in the cell-free protein synthesis system is from 10% to 70% (v/v), wherein the concentration of the DNA or RNA encoding the protein of interest in the cell-free protein synthesis system is from 1 to 100 μg/mL, wherein the concentration of free amino acids in the cell-free protein synthesis system is less than 2 mM, wherein the concentration of free NADH/NADPH in the cell-free protein synthesis system is less than 0.4 mM, wherein the cell-free protein synthesis system does not comprise tryptone, wherein the cell-free protein synthesis system does not comprise yeast extract, and wherein the minimal media comprises at least one of (a) a carbon source; (b) a ammonium source; (c) a magnesium source; (d) a phosphate source; (e) a sulfur source; (f) a trace mineral source; and (g) sodium succinate, wherein the carbon source is chosen from D-glucose and glycerol, and wherein the concentration of the D-glucose in the minimal media is from 0.1% to 2.5% (w/v).

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the system produces the protein of interest when maintained at the temperature, wherein the minimal media comprises at least one of (a) a carbon source; (b) a ammonium source; (c) a magnesium source; (d) a phosphate source; (e) a sulfur source; (f) a trace mineral source; and (g) sodium succinate, wherein the carbon source is chosen from D-glucose and glycerol, and wherein the concentration of the glycerol in the minimal media is from 0.25% to 25% (w/v).

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the system produces the protein of interest when maintained at the temperature, wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5, wherein the temperature is less than about 30° C., wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, wherein the molecular crowder, if present, is chosen from PEG 400-8000, maltodextrin, Ficoll 70-400, dextran, and serum albumin, optionally bovine serum albumin, wherein the concentration of the molecular crowder in the cell-free protein synthesis system is from about 0 to 40 g/L, wherein the carbon source, if present, is chosen from glucose, fructose, galactose, sucrose, lactose, gluconate, starch, maltodextrin, and maltose, wherein the concentration of the carbon source in the cell-free protein synthesis system is from about 0 to 50 g/L, wherein the concentration of the ammonium source, if present, in the cell-free protein synthesis system is from 2 to 20 mM, wherein the potassium source, if present, is chosen from potassium glutamate and potassium gluconate, wherein the concentration of the potassium glutamate in the cell-free protein synthesis system is from about 10 to 270 mM, wherein the concentration of the potassium gluconate in the cell-free protein synthesis system is from about 10 to 250 mM, wherein the concentration of the magnesium source, if present, in the cell-free protein synthesis system is from 0 to about 10 mM, wherein the concentration of the phosphate source, if present, in the cell-free protein synthesis system is from about 5 to about 23.5 mM, wherein the concentration of the sulfur source, if present, in the cell-free protein synthesis system is from 2 to 20 mM, and wherein the concentration of the buffer in the cell-free protein synthesis system is from 50 to 300 mM, wherein the concentration of the cell-free cell extract in the cell-free protein synthesis system is from 10% to 70% (v/v), wherein the concentration of the DNA or RNA encoding the protein of interest in the cell-free protein synthesis system is from 1 to 100 μg/mL, wherein the concentration of free amino acids in the cell-free protein synthesis system is less than 2 mM, wherein the concentration of free NADH/NADPH in the cell-free protein synthesis system is less than 0.4 mM, wherein the cell-free protein synthesis system does not comprise tryptone, wherein the cell-free protein synthesis system does not comprise yeast extract, and wherein the minimal media comprises at least one of (a) a carbon source; (b) a ammonium source; (c) a magnesium source; (d) a phosphate source; (e) a sulfur source; (f) a trace mineral source; and (g) sodium succinate, wherein the carbon source is chosen from D-glucose and glycerol, wherein the concentration of the D-glucose in the minimal media is from 0.1% to 2.5% (w/v), and wherein the concentration of the glycerol in the minimal media is from 0.25% to 25% (w/v).

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the minimal media comprises at least one of (a) a carbon source; (b) a ammonium source; (c) a magnesium source; (d) a phosphate source; (e) a sulfur source; (f) a trace mineral source; and (g) sodium succinate, and wherein the concentration of the sodium succinate in the minimal media is from 0.05% to 5% (w/v).

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the system produces the protein of interest when maintained at the temperature, wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5, wherein the temperature is less than about 30° C., wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, wherein the molecular crowder, if present, is chosen from PEG 400-8000, maltodextrin, Ficoll 70-400, dextran, and serum albumin, optionally bovine serum albumin, wherein the concentration of the molecular crowder in the cell-free protein synthesis system is from about 0 to 40 g/L, wherein the carbon source, if present, is chosen from glucose, fructose, galactose, sucrose, lactose, gluconate, starch, maltodextrin, and maltose, wherein the concentration of the carbon source, if present, in the cell-free protein synthesis system is from about 0 to 50 g/L, wherein the concentration of the ammonium source, if present, in the cell-free protein synthesis system is from 2 to 20 mM, wherein the potassium source, if present, is chosen from potassium glutamate and potassium gluconate, wherein the concentration of the potassium glutamate in the cell-free protein synthesis system is from about 10 to 270 mM, wherein the concentration of the potassium gluconate in the cell-free protein synthesis system is from about 10 to 250 mM, wherein the concentration of the magnesium source, if present, in the cell-free protein synthesis system is from 0 to about 10 mM, wherein the concentration of the phosphate source, if present, in the cell-free protein synthesis system is from about 5 to about 23.5 mM, wherein the concentration of the sulfur source, if present, in the cell-free protein synthesis system is from 2 to 20 mM, and wherein the concentration of the buffer in the cell-free protein synthesis system is from 50 to 300 mM, wherein the concentration of the cell-free cell extract in the cell-free protein synthesis system is from 10% to 70% (v/v), wherein the concentration of the DNA or RNA encoding the protein of interest in the cell-free protein synthesis system is from 1 to 100 μg/mL, wherein the concentration of free amino acids in the cell-free protein synthesis system is less than 2 mM, wherein the concentration of free NADH/NADPH in the cell-free protein synthesis system is less than 0.4 mM, wherein the cell-free protein synthesis system does not comprise tryptone, wherein the cell-free protein synthesis system does not comprise yeast extract, and wherein the minimal media comprises at least one of (a) a carbon source; (b) a ammonium source; (c) a magnesium source; (d) a phosphate source; (e) a sulfur source; (f) a trace mineral source; and (g) sodium succinate, wherein the carbon source is chosen from D-glucose and glycerol, wherein the concentration of the D-glucose in the minimal media is from 0.10% to 2.5% (w/v), wherein the concentration of the glycerol in the minimal media is from 0.25% to 25% (w/v), and wherein the concentration of the sodium succinate in the minimal media is from 0.05% to 50% (w/v).

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the minimal media comprises at least one of (a) a carbon source; (b) a ammonium source; (c) a magnesium source; (d) a phosphate source; (e) a sulfur source; (f) a trace mineral source; and (g) sodium succinate, wherein the phosphate source comprises a sodium phosphate buffer or a potassium phosphate buffer, and wherein the concentration of the potassium phosphate buffer in the minimal media is from 5 to 250 mM.

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the system produces the protein of interest when maintained at the temperature, wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5, wherein the temperature is less than about 30° C., wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, wherein the molecular crowder, if present, is chosen from PEG 400-8000, maltodextrin, Ficoll 70-400, dextran, and serum albumin, optionally bovine serum albumin, wherein the concentration of the molecular crowder in the cell-free protein synthesis system is from about 0 to 40 g/L, wherein the carbon source, if present, is chosen from glucose, fructose, galactose, sucrose, lactose, gluconate, starch, maltodextrin, and maltose, wherein the concentration of the carbon source in the cell-free protein synthesis system is from about 0 to 50 g/L, wherein the concentration of the ammonium source, if present, in the cell-free protein synthesis system is from 2 to 20 mM, wherein the potassium source, if present, is chosen from potassium glutamate and potassium gluconate, wherein the concentration of the potassium glutamate in the cell-free protein synthesis system is from about 10 to 270 mM, wherein the concentration of the potassium gluconate in the cell-free protein synthesis system is from about 10 to 250 mM, wherein the concentration of the magnesium source, if present, in the cell-free protein synthesis system is from 0 to about 10 mM, wherein the concentration of the phosphate source, if present, in the cell-free protein synthesis system is from about 5 to about 23.5 mM, wherein the concentration of the sulfur source, if present, in the cell-free protein synthesis system is from 2 to 20 mM, and wherein the concentration of the buffer in the cell-free protein synthesis system is from 50 to 300 mM, wherein the concentration of the cell-free cell extract in the cell-free protein synthesis system is from 10% to 70% (v/v), wherein the concentration of the DNA or RNA encoding the protein of interest in the cell-free protein synthesis system is from 1 to 100 μg/mL, wherein the concentration of free amino acids in the cell-free protein synthesis system is less than 2 mM, wherein the concentration of free NADH/NADPH in the cell-free protein synthesis system is less than 0.4 mM, wherein the cell-free protein synthesis system does not comprise tryptone, wherein the cell-free protein synthesis system does not comprise yeast extract, and wherein the minimal media comprises at least one of (a) a carbon source; (b) a ammonium source; (c) a magnesium source; (d) a phosphate source; (e) a sulfur source; (f) a trace mineral source; and (g) sodium succinate, wherein the carbon source is chosen from D-glucose and glycerol, wherein the concentration of the D-glucose in the minimal media is from 0.1% to 2.5% (w/v), wherein the concentration of the glycerol in the minimal media is from 0.25% to 25% (w/v), wherein the concentration of the sodium succinate in the minimal media is from 0.05% to 5% (w/v), wherein the phosphate source comprises a sodium phosphate buffer or a potassium phosphate buffer, and wherein the concentration of the potassium phosphate buffer in the minimal media is from 5 to 250 mM.

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the system produces the protein of interest when maintained at the temperature, wherein the minimal media comprises at least one of (a) a carbon source; (b) a ammonium source; (c) a magnesium source; (d) a phosphate source; (e) a sulfur source; (f) a trace mineral source; and (g) sodium succinate, wherein the phosphate source comprises a sodium phosphate buffer or a potassium phosphate buffer, and wherein the pH of the potassium phosphate buffer in the minimal media is from 6.7 to 7.7.

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the system produces the protein of interest when maintained at the temperature, wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5, wherein the temperature is less than about 30° C., wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, wherein the molecular crowder, if present, is chosen from PEG 400-8000, maltodextrin, Ficoll 70-400, dextran, and serum albumin, optionally bovine serum albumin, wherein the concentration of the molecular crowder in the cell-free protein synthesis system is from about 0 to 40 g/L, wherein the carbon source, if present, is chosen from glucose, fructose, galactose, sucrose, lactose, gluconate, starch, maltodextrin, and maltose, wherein the concentration of the carbon source, if present, in the cell-free protein synthesis system is from about 0 to 50 g/L, wherein the concentration of the ammonium source, if present, in the cell-free protein synthesis system is from 2 to 20 mM, wherein the potassium source, if present, is chosen from potassium glutamate and potassium gluconate, wherein the concentration of the potassium glutamate in the cell-free protein synthesis system is from about 10 to 270 mM, wherein the concentration of the potassium gluconate in the cell-free protein synthesis system is from about 10 to 250 mM, wherein the concentration of the magnesium source, if present, in the cell-free protein synthesis system is from 0 to about 10 mM, wherein the concentration of the phosphate source, if present, in the cell-free protein synthesis system is from about 5 to about 23.5 mM, wherein the concentration of the sulfur source, if present, in the cell-free protein synthesis system is from 2 to 20 mM, and wherein the concentration of the buffer in the cell-free protein synthesis system is from 50 to 300 mM, wherein the concentration of the cell-free cell extract in the cell-free protein synthesis system is from 10% to 70% (v/v), wherein the concentration of the DNA or RNA encoding the protein of interest in the cell-free protein synthesis system is from 1 to 100 μg/mL, wherein the concentration of free amino acids in the cell-free protein synthesis system is less than 2 mM, wherein the concentration of free NADH/NADPH in the cell-free protein synthesis system is less than 0.4 mM, wherein the cell-free protein synthesis system does not comprise tryptone, wherein the cell-free protein synthesis system does not comprise yeast extract, and wherein the minimal media comprises at least one of (a) a carbon source; (b) a ammonium source; (c) a magnesium source; (d) a phosphate source; (e) a sulfur source; (f) a trace mineral source; and (g) sodium succinate, wherein the carbon source is chosen from D-glucose and glycerol, wherein the concentration of the D-glucose in the minimal media is from 0.10% to 2.5% (w/v), wherein the concentration of the glycerol in the minimal media is from 0.25% to 25% (w/v), wherein the concentration of the sodium succinate in the minimal media is from 0.05% to 5% (w/v), wherein the phosphate source comprises a sodium phosphate buffer or a potassium phosphate buffer, wherein the concentration of the potassium phosphate buffer in the minimal media is from 5 to 250 mM, and wherein the pH of the potassium phosphate buffer in the minimal media is from 6.7 to 7.7.

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the system produces the protein of interest when maintained at the temperature, wherein the minimal media comprises at least one of (a) a carbon source; (b) a ammonium source; (c) a magnesium source; (d) a phosphate source; (e) a sulfur source; (f) a trace mineral source; and (g) sodium succinate, wherein the ammonium source comprises ammonium chloride, and wherein the concentration of the ammonium chloride in the minimal media is from 5 to 250 mM.

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the system produces the protein of interest when maintained at the temperature, wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5, wherein the temperature is less than about 30° C., wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, wherein the molecular crowder, if present, is chosen from PEG 400-8000, maltodextrin, Ficoll 70-400, dextran, and serum albumin, optionally bovine serum albumin, wherein the concentration of the molecular crowder in the cell-free protein synthesis system is from about 0 to 40 g/L, wherein the carbon source, if present, is chosen from glucose, fructose, galactose, sucrose, lactose, gluconate, starch, maltodextrin, and maltose, wherein the concentration of the carbon source, if present, in the cell-free protein synthesis system is from about 0 to 50 g/L, wherein the concentration of the ammonium source, if present, in the cell-free protein synthesis system is from 2 to 20 mM, wherein the potassium source, if present, is chosen from potassium glutamate and potassium gluconate, wherein the concentration of the potassium glutamate in the cell-free protein synthesis system is from about 10 to 270 mM, wherein the concentration of the potassium gluconate in the cell-free protein synthesis system is from about 10 to 250 mM, wherein the concentration of the magnesium source, if present, in the cell-free protein synthesis system is from 0 to about 10 mM, wherein the concentration of the phosphate source, if present, in the cell-free protein synthesis system is from about 5 to about 23.5 mM, wherein the concentration of the sulfur source, if present, in the cell-free protein synthesis system is from 2 to 20 mM, and wherein the concentration of the buffer in the cell-free protein synthesis system is from 50 to 300 mM, wherein the concentration of the cell-free cell extract in the cell-free protein synthesis system is from 10% to 70% (v/v), wherein the concentration of the DNA or RNA encoding the protein of interest in the cell-free protein synthesis system is from 1 to 100 μg/mL, wherein the concentration of free amino acids in the cell-free protein synthesis system is less than 2 mM, wherein the concentration of free NADH/NADPH in the cell-free protein synthesis system is less than 0.4 mM, wherein the cell-free protein synthesis system does not comprise tryptone, wherein the cell-free protein synthesis system does not comprise yeast extract, and wherein the minimal media comprises at least one of (a) a carbon source; (b) a ammonium source; (c) a magnesium source; (d) a phosphate source; (e) a sulfur source; (f) a trace mineral source; and (g) sodium succinate, wherein the carbon source is chosen from D-glucose and glycerol, wherein the concentration of the D-glucose in the minimal media is from 0.10% to 2.5% (w/v), wherein the concentration of the glycerol in the minimal media is from 0.25% to 25% (w/v), wherein the concentration of the sodium succinate in the minimal media is from 0.05% to 5% (w/v), wherein the phosphate source comprises a sodium phosphate buffer or a potassium phosphate buffer, wherein the concentration of the potassium phosphate buffer in the minimal media is from 5 to 250 mM, wherein the pH of the potassium phosphate buffer in the minimal media is from 6.7 to 7.7, wherein the ammonium source comprises ammonium chloride, and wherein the concentration of the ammonium chloride in the minimal media is from 5 to 250 mM.

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the system produces the protein of interest when maintained at the temperature, wherein the minimal media comprises at least one of (a) a carbon source; (b) a ammonium source; (c) a magnesium source; (d) a phosphate source; (e) a sulfur source; (f) a trace mineral source; and (g) sodium succinate, wherein the sulfur source comprises sodium sulfate, and wherein the concentration of the sodium sulfate in the minimal media is from 0.5 to 25 mM.

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the system produces the protein of interest when maintained at the temperature, wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5, wherein the temperature is less than about 30° C., wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, wherein the molecular crowder, if present, is chosen from PEG 400-8000, maltodextrin, Ficoll 70-400, dextran, and serum albumin, optionally bovine serum albumin, wherein the concentration of the molecular crowder in the cell-free protein synthesis system is from about 0 to 40 g/L, wherein the carbon source, if present, is chosen from glucose, fructose, galactose, sucrose, lactose, gluconate, starch, maltodextrin, and maltose, wherein the concentration of the carbon source, if present, in the cell-free protein synthesis system is from about 0 to 50 g/L, wherein the concentration of the ammonium source, if present, in the cell-free protein synthesis system is from 2 to 20 mM, wherein the potassium source, if present, is chosen from potassium glutamate and potassium gluconate, wherein the concentration of the potassium glutamate in the cell-free protein synthesis system is from about 10 to 270 mM, wherein the concentration of the potassium gluconate in the cell-free protein synthesis system is from about 10 to 250 mM, wherein the concentration of the magnesium source, if present, in the cell-free protein synthesis system is from 0 to about 10 mM, wherein the concentration of the phosphate source, if present, in the cell-free protein synthesis system is from about 5 to about 23.5 mM, wherein the concentration of the sulfur source, if present, in the cell-free protein synthesis system is from 2 to 20 mM, and wherein the concentration of the buffer in the cell-free protein synthesis system is from 50 to 300 mM, wherein the concentration of the cell-free cell extract in the cell-free protein synthesis system is from 10% to 70% (v/v), wherein the concentration of the DNA or RNA encoding the protein of interest in the cell-free protein synthesis system is from 1 to 100 μg/mL, wherein the concentration of free amino acids in the cell-free protein synthesis system is less than 2 mM, wherein the concentration of free NADH/NADPH in the cell-free protein synthesis system is less than 0.4 mM, wherein the cell-free protein synthesis system does not comprise tryptone, wherein the cell-free protein synthesis system does not comprise yeast extract, and wherein the minimal media comprises at least one of (a) a carbon source; (b) a ammonium source; (c) a magnesium source; (d) a phosphate source; (e) a sulfur source; (f) a trace mineral source; and (g) sodium succinate, wherein the carbon source is chosen from D-glucose and glycerol, wherein the concentration of the D-glucose in the minimal media is from 0.10% to 2.5% (w/v), wherein the concentration of the glycerol in the minimal media is from 0.25% to 25% (w/v), wherein the concentration of the sodium succinate in the minimal media is from 0.05% to 5% (w/v), wherein the phosphate source comprises a sodium phosphate buffer or a potassium phosphate buffer, wherein the concentration of the potassium phosphate buffer in the minimal media is from 5 to 250 mM, wherein the pH of the potassium phosphate buffer in the minimal media is from 6.7 to 7.7, wherein the ammonium source comprises ammonium chloride, wherein the concentration of the ammonium chloride in the minimal media is from 5 to 250 mM, wherein the sulfur source comprises sodium sulfate, and wherein the concentration of the sodium sulfate in the minimal media is from 0.5 to 25 mM.

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the system produces the protein of interest when maintained at the temperature, wherein the minimal media comprises at least one of (a) a carbon source; (b) a ammonium source; (c) a magnesium source; (d) a phosphate source; (e) a sulfur source; (f) a trace mineral source; and (g) sodium succinate, wherein the magnesium source comprises magnesium sulfate heptahydrate, and wherein the concentration of the magnesium sulfate heptahydrate in the minimal media is from 0.2 to 20 mM.

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the system produces the protein of interest when maintained at the temperature, wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5, wherein the temperature is less than about 30° C., wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, wherein the molecular crowder, if present, is chosen from PEG 400-8000, maltodextrin, Ficoll 70-400, dextran, and serum albumin, optionally bovine serum albumin, wherein the concentration of the molecular crowder in the cell-free protein synthesis system is from about 0 to 40 g/L, wherein the carbon source, if present, is chosen from glucose, fructose, galactose, sucrose, lactose, gluconate, starch, maltodextrin, and maltose, wherein the concentration of the carbon source, if present, in the cell-free protein synthesis system is from about 0 to 50 g/L, wherein the concentration of the ammonium source, if present, in the cell-free protein synthesis system is from 2 to 20 mM, wherein the potassium source, if present, is chosen from potassium glutamate and potassium gluconate, wherein the concentration of the potassium glutamate in the cell-free protein synthesis system is from about 10 to 270 mM, wherein the concentration of the potassium gluconate in the cell-free protein synthesis system is from about 10 to 250 mM, wherein the concentration of the magnesium source, if present, in the cell-free protein synthesis system is from 0 to about 10 mM, wherein the concentration of the phosphate source, if present, in the cell-free protein synthesis system is from about 5 to about 23.5 mM, wherein the concentration of the sulfur source, if present, in the cell-free protein synthesis system is from 2 to 20 mM, and wherein the concentration of the buffer in the cell-free protein synthesis system is from 50 to 300 mM, wherein the concentration of the cell-free cell extract in the cell-free protein synthesis system is from 10% to 70% (v/v), wherein the concentration of the DNA or RNA encoding the protein of interest in the cell-free protein synthesis system is from 1 to 100 μg/mL, wherein the concentration of free amino acids in the cell-free protein synthesis system is less than 2 mM, wherein the concentration of free NADH/NADPH in the cell-free protein synthesis system is less than 0.4 mM, wherein the cell-free protein synthesis system does not comprise tryptone, wherein the cell-free protein synthesis system does not comprise yeast extract, and wherein the minimal media comprises at least one of (a) a carbon source; (b) a ammonium source; (c) a magnesium source; (d) a phosphate source; (e) a sulfur source; (f) a trace mineral source; and (g) sodium succinate, wherein the carbon source is chosen from D-glucose and glycerol, wherein the concentration of the D-glucose in the minimal media is from 0.1% to 2.5% (w/v), wherein the concentration of the glycerol in the minimal media is from 0.25% to 25% (w/v), wherein the concentration of the sodium succinate in the minimal media is from 0.05% to 5% (w/v), wherein the phosphate source comprises a sodium phosphate buffer or a potassium phosphate buffer, wherein the concentration of the potassium phosphate buffer in the minimal media is from 5 to 250 mM, wherein the pH of the potassium phosphate buffer in the minimal media is from 6.7 to 7.7, wherein the ammonium source comprises ammonium chloride, wherein the concentration of the ammonium chloride in the minimal media is from 5 to 250 mM, wherein the sulfur source comprises sodium sulfate, wherein the concentration of the sodium sulfate in the minimal media is from 0.5 to 25 mM, wherein the magnesium source comprises magnesium sulfate heptahydrate, and wherein the concentration of the magnesium sulfate heptahydrate in the minimal media is from 0.2 to 20 mM.

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the system produces the protein of interest when maintained at the temperature, wherein the minimal media comprises at least one of (a) a carbon source; (b) a ammonium source; (c) a magnesium source; (d) a phosphate source; (e) a sulfur source; (f) a trace mineral source; and (g) sodium succinate, wherein the trace mineral source is chosen from an iron source, a copper source, a nickel source, a zinc source, a molybdenum source, a boron source, and a manganese source, wherein the iron source comprises ferric citrate, and wherein the concentration of the ferric citrate in the minimal media is from 10 to 1,000 μM.

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the system produces the protein of interest when maintained at the temperature, wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5, wherein the temperature is less than about 30° C., wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, wherein the molecular crowder, if present, is chosen from PEG 400-8000, maltodextrin, Ficoll 70-400, dextran, and serum albumin, optionally bovine serum albumin, wherein the concentration of the molecular crowder in the cell-free protein synthesis system is from about 0 to 40 g/L, wherein the carbon source, if present, is chosen from glucose, fructose, galactose, sucrose, lactose, gluconate, starch, maltodextrin, and maltose, wherein the concentration of the carbon source, if present, in the cell-free protein synthesis system is from about 0 to 50 g/L, wherein the concentration of the ammonium source, if present, in the cell-free protein synthesis system is from 2 to 20 mM, wherein the potassium source, if present, is chosen from potassium glutamate and potassium gluconate, wherein the concentration of the potassium glutamate in the cell-free protein synthesis system is from about 10 to 270 mM, wherein the concentration of the potassium gluconate in the cell-free protein synthesis system is from about 10 to 250 mM, wherein the concentration of the magnesium source, if present, in the cell-free protein synthesis system is from 0 to about 10 mM, wherein the concentration of the phosphate source, if present, in the cell-free protein synthesis system is from about 5 to about 23.5 mM, wherein the concentration of the sulfur source, if present, in the cell-free protein synthesis system is from 2 to 20 mM, and wherein the concentration of the buffer in the cell-free protein synthesis system is from 50 to 300 mM, wherein the concentration of the cell-free cell extract in the cell-free protein synthesis system is from 10% to 70% (v/v), wherein the concentration of the DNA or RNA encoding the protein of interest in the cell-free protein synthesis system is from 1 to 100 μg/mL, wherein the concentration of free amino acids in the cell-free protein synthesis system is less than 2 mM, wherein the concentration of free NADH/NADPH in the cell-free protein synthesis system is less than 0.4 mM, wherein the cell-free protein synthesis system does not comprise tryptone, wherein the cell-free protein synthesis system does not comprise yeast extract, and wherein the minimal media comprises at least one of (a) a carbon source; (b) a ammonium source; (c) a magnesium source; (d) a phosphate source; (e) a sulfur source; (f) a trace mineral source; and (g) sodium succinate, wherein the carbon source is chosen from D-glucose and glycerol, wherein the concentration of the D-glucose in the minimal media is from 0.10% to 2.5% (w/v), wherein the concentration of the glycerol in the minimal media is from 0.25% to 25% (w/v), wherein the concentration of the sodium succinate in the minimal media is from 0.05% to 5% (w/v), wherein the phosphate source comprises a sodium phosphate buffer or a potassium phosphate buffer, wherein the concentration of the potassium phosphate buffer in the minimal media is from 5 to 250 mM, wherein the pH of the potassium phosphate buffer in the minimal media is from 6.7 to 7.7, wherein the ammonium source comprises ammonium chloride, wherein the concentration of the ammonium chloride in the minimal media is from 5 to 250 mM, wherein the sulfur source comprises sodium sulfate, wherein the concentration of the sodium sulfate in the minimal media is from 0.5 to 25 mM, wherein the magnesium source comprises magnesium sulfate heptahydrate, wherein the concentration of the magnesium sulfate heptahydrate in the minimal media is from 0.2 to 20 mM, wherein the trace mineral source is chosen from an iron source, a copper source, a nickel source, a zinc source, a molybdenum source, a boron source, and a manganese source, wherein the iron source comprises ferric citrate, and wherein the concentration of the ferric citrate in the minimal media is from 10 to 1,000 M.

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the system produces the protein of interest when maintained at the temperature, and wherein the bacteria are Escherichia coli (E. coli), Bacillus subtilis (B. subtilis), or Vibrio natriegens (V. natriegens).

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the system produces the protein of interest when maintained at the temperature, wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5, wherein the temperature is less than about 30° C., wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, wherein the molecular crowder, if present, is chosen from PEG 400-8000, maltodextrin, Ficoll 70-400, dextran, and serum albumin, optionally bovine serum albumin, wherein the concentration of the molecular crowder in the cell-free protein synthesis system is from about 0 to 40 g/L, wherein the carbon source, if present, is chosen from glucose, fructose, galactose, sucrose, lactose, gluconate, starch, maltodextrin, and maltose, wherein the concentration of the carbon source, if present, in the cell-free protein synthesis system is from about 0 to 50 g/L, wherein the concentration of the ammonium source, if present, in the cell-free protein synthesis system is from 2 to 20 mM, wherein the potassium source, if present, is chosen from potassium glutamate and potassium gluconate, wherein the concentration of the potassium glutamate in the cell-free protein synthesis system is from about 10 to 270 mM, wherein the concentration of the potassium gluconate in the cell-free protein synthesis system is from about 10 to 250 mM, wherein the concentration of the magnesium source, if present, in the cell-free protein synthesis system is from 0 to about 10 mM, wherein the concentration of the phosphate source, if present, in the cell-free protein synthesis system is from about 5 to about 23.5 mM, wherein the concentration of the sulfur source, if present, in the cell-free protein synthesis system is from 2 to 20 mM, and wherein the concentration of the buffer in the cell-free protein synthesis system is from 50 to 300 mM, wherein the concentration of the cell-free cell extract in the cell-free protein synthesis system is from 10% to 70% (v/v), wherein the concentration of the DNA or RNA encoding the protein of interest in the cell-free protein synthesis system is from 1 to 100 μg/mL, wherein the concentration of free amino acids in the cell-free protein synthesis system is less than 2 mM, wherein the concentration of free NADH/NADPH in the cell-free protein synthesis system is less than 0.4 mM, wherein the cell-free protein synthesis system does not comprise tryptone, wherein the cell-free protein synthesis system does not comprise yeast extract, and wherein the minimal media comprises at least one of (a) a carbon source; (b) a ammonium source; (c) a magnesium source; (d) a phosphate source; (e) a sulfur source; (f) a trace mineral source; and (g) sodium succinate, wherein the carbon source is chosen from D-glucose and glycerol, wherein the concentration of the D-glucose in the minimal media is from 0.1% to 2.5% (w/v), wherein the concentration of the glycerol in the minimal media is from 0.25% to 25% (w/v), wherein the concentration of the sodium succinate in the minimal media is from 0.05% to 5% (w/v), wherein the phosphate source comprises a sodium phosphate buffer or a potassium phosphate buffer, wherein the concentration of the potassium phosphate buffer in the minimal media is from 5 to 250 mM, wherein the pH of the potassium phosphate buffer in the minimal media is from 6.7 to 7.7, wherein the ammonium source comprises ammonium chloride, wherein the concentration of the ammonium chloride in the minimal media is from 5 to 250 mM, wherein the sulfur source comprises sodium sulfate, wherein the concentration of the sodium sulfate in the minimal media is from 0.5 to 25 mM, wherein the magnesium source comprises magnesium sulfate heptahydrate, wherein the concentration of the magnesium sulfate heptahydrate in the minimal media is from 0.2 to 20 mM, wherein the trace mineral source is chosen from an iron source, a copper source, a nickel source, a zinc source, a molybdenum source, a boron source, and a manganese source, wherein the iron source comprises ferric citrate, wherein the concentration of the ferric citrate in the minimal media is from 10 to 1,000 M, and wherein the bacteria are Escherichia coli (E. coli), Bacillus subtilis (B. subtilis), or Vibrio natriegens (V. natriegens).

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the system produces the protein of interest when maintained at the temperature, and wherein the cell-free cell extract is prepared by preparing a suspension in which each one gram of the bacteria is suspended in 1.1 mL of a suspension buffer.

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the system produces the protein of interest when maintained at the temperature, wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5, wherein the temperature is less than about 30° C., wherein the reaction mixture further comprises at least one of (a) a molecular crowder; (b) a carbon source; (c) a ammonium source; (d) a potassium source; (e) a magnesium source; (f) a phosphate source; and (g) a sulfur source; and (h) a buffer, wherein the molecular crowder, if present, is chosen from PEG 400-8000, maltodextrin, Ficoll 70-400, dextran, and serum albumin, optionally bovine serum albumin, wherein the concentration of the molecular crowder in the cell-free protein synthesis system is from about 0 to 40 g/L, wherein the carbon source, if present, is chosen from glucose, fructose, galactose, sucrose, lactose, gluconate, starch, maltodextrin, and maltose, wherein the concentration of the carbon source, if present, in the cell-free protein synthesis system is from about 0 to 50 g/L, wherein the concentration of the ammonium source, if present, in the cell-free protein synthesis system is from 2 to 20 mM, wherein the potassium source, if present, is chosen from potassium glutamate and potassium gluconate, wherein the concentration of the potassium glutamate in the cell-free protein synthesis system is from about 10 to 270 mM, wherein the concentration of the potassium gluconate in the cell-free protein synthesis system is from about 10 to 250 mM, wherein the concentration of the magnesium source, if present, in the cell-free protein synthesis system is from 0 to about 10 mM, wherein the concentration of the phosphate source, if present, in the cell-free protein synthesis system is from about 5 to about 23.5 mM, wherein the concentration of the sulfur source, if present, in the cell-free protein synthesis system is from 2 to 20 mM, and wherein the concentration of the buffer in the cell-free protein synthesis system is from 50 to 300 mM, wherein the concentration of the cell-free cell extract in the cell-free protein synthesis system is from 10% to 70% (v/v), wherein the concentration of the DNA or RNA encoding the protein of interest in the cell-free protein synthesis system is from 1 to 100 μg/mL, wherein the concentration of free amino acids in the cell-free protein synthesis system is less than 2 mM, wherein the concentration of free NADH/NADPH in the cell-free protein synthesis system is less than 0.4 mM, wherein the cell-free protein synthesis system does not comprise tryptone, wherein the cell-free protein synthesis system does not comprise yeast extract, and wherein the minimal media comprises at least one of (a) a carbon source; (b) a ammonium source; (c) a magnesium source; (d) a phosphate source; (e) a sulfur source; (f) a trace mineral source; and (g) sodium succinate, wherein the carbon source is chosen from D-glucose and glycerol, wherein the concentration of the D-glucose in the minimal media is from 0.10% to 2.5% (w/v), wherein the concentration of the glycerol in the minimal media is from 0.25% to 25% (w/v), wherein the concentration of the sodium succinate in the minimal media is from 0.05% to 5% (w/v), wherein the phosphate source comprises a sodium phosphate buffer or a potassium phosphate buffer, wherein the concentration of the potassium phosphate buffer in the minimal media is from 5 to 250 mM, wherein the pH of the potassium phosphate buffer in the minimal media is from 6.7 to 7.7, wherein the ammonium source comprises ammonium chloride, wherein the concentration of the ammonium chloride in the minimal media is from 5 to 250 mM, wherein the sulfur source comprises sodium sulfate, wherein the concentration of the sodium sulfate in the minimal media is from 0.5 to 25 mM, wherein the magnesium source comprises magnesium sulfate heptahydrate, wherein the concentration of the magnesium sulfate heptahydrate in the minimal media is from 0.2 to 20 mM, wherein the trace mineral source is chosen from an iron source, a copper source, a nickel source, a zinc source, a molybdenum source, a boron source, and a manganese source, wherein the iron source comprises ferric citrate, wherein the concentration of the ferric citrate in the minimal media is from 10 to 1,000 M, wherein the bacteria are Escherichia coli (E. coli), Bacillus subtilis (B. subtilis), or Vibrio natriegens (V. natriegens), and wherein the cell-free cell extract is prepared by preparing a suspension in which each one gram of the bacteria is suspended in 1.1 mL of a suspension buffer.

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the system produces the protein of interest when maintained at the temperature, wherein the cell-free cell extract is prepared by preparing a suspension in which each one gram of the bacteria is suspended in 1.1 mL of a suspension buffer, wherein the cell-free cell extract is prepared by lysing the bacteria in the suspension, and wherein the lysing comprises sonication.

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the system produces the protein of interest when maintained at the temperature, wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5, wherein the temperature is less than about 30° C., wherein the reaction mixture further comprises at least one of (a) a molecular crowder; (b) a carbon source; (c) a ammonium source; (d) a potassium source; (e) a magnesium source; (f) a phosphate source; and (g) a sulfur source; and (h) a buffer, wherein the molecular crowder, if present, is chosen from PEG 400-8000, maltodextrin, Ficoll 70-400, dextran, and serum albumin, optionally bovine serum albumin, wherein the concentration of the molecular crowder in the cell-free protein synthesis system is from about 0 to 40 g/L, wherein the carbon source, if present, is chosen from glucose, fructose, galactose, sucrose, lactose, gluconate, starch, maltodextrin, and maltose, wherein the concentration of the carbon source, if present, in the cell-free protein synthesis system is from about 0 to 50 g/L, wherein the concentration of the ammonium source, if present, in the cell-free protein synthesis system is from 2 to 20 mM, wherein the potassium source, if present, is chosen from potassium glutamate and potassium gluconate, wherein the concentration of the potassium glutamate in the cell-free protein synthesis system is from about 10 to 270 mM, wherein the concentration of the potassium gluconate in the cell-free protein synthesis system is from about 10 to 250 mM, wherein the concentration of the magnesium source, if present, in the cell-free protein synthesis system is from 0 to about 10 mM, wherein the concentration of the phosphate source, if present, in the cell-free protein synthesis system is from about 5 to about 23.5 mM, wherein the concentration of the sulfur source, if present, in the cell-free protein synthesis system is from 2 to 20 mM, and wherein the concentration of the buffer in the cell-free protein synthesis system is from 50 to 300 mM, wherein the concentration of the cell-free cell extract in the cell-free protein synthesis system is from 10% to 70% (v/v), wherein the concentration of the DNA or RNA encoding the protein of interest in the cell-free protein synthesis system is from 1 to 100 μg/mL, wherein the concentration of free amino acids in the cell-free protein synthesis system is less than 2 mM, wherein the concentration of free NADH/NADPH in the cell-free protein synthesis system is less than 0.4 mM, wherein the cell-free protein synthesis system does not comprise tryptone, wherein the cell-free protein synthesis system does not comprise yeast extract, and wherein the minimal media comprises at least one of (a) a carbon source; (b) a ammonium source; (c) a magnesium source; (d) a phosphate source; (e) a sulfur source; (f) a trace mineral source; and (g) sodium succinate, wherein the carbon source is chosen from D-glucose and glycerol, wherein the concentration of the D-glucose in the minimal media is from 0.10% to 2.5% (w/v), wherein the concentration of the glycerol in the minimal media is from 0.25% to 25% (w/v), wherein the concentration of the sodium succinate in the minimal media is from 0.05% to 5% (w/v), wherein the phosphate source comprises a sodium phosphate buffer or a potassium phosphate buffer, wherein the concentration of the potassium phosphate buffer in the minimal media is from 5 to 250 mM, wherein the pH of the potassium phosphate buffer in the minimal media is from 6.7 to 7.7, wherein the ammonium source comprises ammonium chloride, wherein the concentration of the ammonium chloride in the minimal media is from 5 to 250 mM, wherein the sulfur source comprises sodium sulfate, wherein the concentration of the sodium sulfate in the minimal media is from 0.5 to 25 mM, wherein the magnesium source comprises magnesium sulfate heptahydrate, wherein the concentration of the magnesium sulfate heptahydrate in the minimal media is from 0.2 to 20 mM, wherein the trace mineral source is chosen from an iron source, a copper source, a nickel source, a zinc source, a molybdenum source, a boron source, and a manganese source, wherein the iron source comprises ferric citrate, wherein the concentration of the ferric citrate in the minimal media is from 10 to 1,000 M, wherein the bacteria are Escherichia coli (E. coli), Bacillus subtilis (B. subtilis), or Vibrio natriegens (V. natriegens), wherein the cell-free cell extract is prepared by preparing a suspension in which each one gram of the bacteria is suspended in 1.1 mL of a suspension buffer, wherein the cell-free cell extract is prepared by lysing the bacteria in the suspension, and wherein the lysing comprises sonication.

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the system produces the protein of interest when maintained at the temperature, wherein the cell-free cell extract is prepared by preparing a suspension in which each one gram of the bacteria is suspended in 1.1 mL of a suspension buffer, and wherein the suspension buffer comprises one or more of (a) Tris-acetate; (b) magnesium glutamate; and (c) potassium glutamate.

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5, wherein the temperature is less than about 30° C., wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, wherein the molecular crowder, if present, is chosen from PEG 400-8000, maltodextrin, Ficoll 70-400, dextran, and serum albumin, optionally bovine serum albumin, wherein the concentration of the molecular crowder in the cell-free protein synthesis system is from about 0 to 40 g/L, wherein the carbon source, if present, is chosen from glucose, fructose, galactose, sucrose, lactose, gluconate, starch, maltodextrin, and maltose, wherein the concentration of the carbon source, if present, in the cell-free protein synthesis system is from about 0 to 50 g/L, wherein the concentration of the ammonium source, if present, in the cell-free protein synthesis system is from 2 to 20 mM, wherein the potassium source, if present, is chosen from potassium glutamate and potassium gluconate, wherein the concentration of the potassium glutamate in the cell-free protein synthesis system is from about 10 to 270 mM, wherein the concentration of the potassium gluconate in the cell-free protein synthesis system is from about 10 to 250 mM, wherein the concentration of the magnesium source, if present, in the cell-free protein synthesis system is from 0 to about 10 mM, wherein the concentration of the phosphate source, if present, in the cell-free protein synthesis system is from about 5 to about 23.5 mM, wherein the concentration of the sulfur source, if present, in the cell-free protein synthesis system is from 2 to 20 mM, and wherein the concentration of the buffer in the cell-free protein synthesis system is from 50 to 300 mM, wherein the concentration of the cell-free cell extract in the cell-free protein synthesis system is from 10% to 70% (v/v), wherein the concentration of the DNA or RNA encoding the protein of interest in the cell-free protein synthesis system is from 1 to 100 μg/mL, wherein the concentration of free amino acids in the cell-free protein synthesis system is less than 2 mM, wherein the concentration of free NADH/NADPH in the cell-free protein synthesis system is less than 0.4 mM, wherein the cell-free protein synthesis system does not comprise tryptone, wherein the cell-free protein synthesis system does not comprise yeast extract, and wherein the minimal media comprises at least one of (a) a carbon source; (b) a ammonium source; (c) a magnesium source; (d) a phosphate source; (e) a sulfur source; (f) a trace mineral source; and (g) sodium succinate, wherein the carbon source is chosen from D-glucose and glycerol, wherein the concentration of the D-glucose in the minimal media is from 0.10% to 2.5% (w/v), wherein the concentration of the glycerol in the minimal media is from 0.25% to 25% (w/v), wherein the concentration of the sodium succinate in the minimal media is from 0.05% to 5% (w/v), wherein the phosphate source comprises a sodium phosphate buffer or a potassium phosphate buffer, wherein the concentration of the potassium phosphate buffer in the minimal media is from 5 to 250 mM, wherein the pH of the potassium phosphate buffer in the minimal media is from 6.7 to 7.7, wherein the ammonium source comprises ammonium chloride, wherein the concentration of the ammonium chloride in the minimal media is from 5 to 250 mM, wherein the sulfur source comprises sodium sulfate, wherein the concentration of the sodium sulfate in the minimal media is from 0.5 to 25 mM, wherein the magnesium source comprises magnesium sulfate heptahydrate, wherein the concentration of the magnesium sulfate heptahydrate in the minimal media is from 0.2 to 20 mM, wherein the trace mineral source is chosen from an iron source, a copper source, a nickel source, a zinc source, a molybdenum source, a boron source, and a manganese source, wherein the iron source comprises ferric citrate, wherein the concentration of the ferric citrate in the minimal media is from 10 to 1,000 M, wherein the bacteria are Escherichia coli (E. coli), Bacillus subtilis (B. subtilis), or Vibrio natriegens (V. natriegens), wherein the cell-free cell extract is prepared by preparing a suspension in which each one gram of the bacteria is suspended in 1.1 mL of a suspension buffer, wherein the cell-free cell extract is prepared by lysing the bacteria in the suspension, wherein the lysing comprises sonication, and wherein the suspension buffer comprises one or more of (a) Tris-acetate; (b) magnesium glutamate; and (c) potassium glutamate.

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the system produces the protein of interest when maintained at the temperature, wherein the cell-free cell extract is prepared by preparing a suspension in which each one gram of the bacteria is suspended in 1.1 mL of a suspension buffer, wherein the suspension buffer comprises one or more of (a) Tris-acetate; (b) magnesium glutamate; and (c) potassium glutamate, and wherein the concentration of the Tris-acetate in the suspension buffer is from 1 to 100 mM.

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5, wherein the temperature is less than about 30° C., wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, wherein the molecular crowder, if present, is chosen from PEG 400-8000, maltodextrin, Ficoll 70-400, dextran, and serum albumin, optionally bovine serum albumin, wherein the concentration of the molecular crowder in the cell-free protein synthesis system is from about 0 to 40 g/L, wherein the carbon source, if present, is chosen from glucose, fructose, galactose, sucrose, lactose, gluconate, starch, maltodextrin, and maltose, wherein the concentration of the carbon source, if present, in the cell-free protein synthesis system is from about 0 to 50 g/L, wherein the concentration of the ammonium source, if present, in the cell-free protein synthesis system is from 2 to 20 mM, wherein the potassium source, if present, is chosen from potassium glutamate and potassium gluconate, wherein the concentration of the potassium glutamate in the cell-free protein synthesis system is from about 10 to 270 mM, wherein the concentration of the potassium gluconate in the cell-free protein synthesis system is from about 10 to 250 mM, wherein the concentration of the magnesium source, if present, in the cell-free protein synthesis system is from 0 to about 10 mM, wherein the concentration of the phosphate source, if present, in the cell-free protein synthesis system is from about 5 to about 23.5 mM, wherein the concentration of the sulfur source, if present, in the cell-free protein synthesis system is from 2 to 20 mM, and wherein the concentration of the buffer in the cell-free protein synthesis system is from 50 to 300 mM, wherein the concentration of the cell-free cell extract in the cell-free protein synthesis system is from 10% to 70% (v/v), wherein the concentration of the DNA or RNA encoding the protein of interest in the cell-free protein synthesis system is from 1 to 100 μg/mL, wherein the concentration of free amino acids in the cell-free protein synthesis system is less than 2 mM, wherein the concentration of free NADH/NADPH in the cell-free protein synthesis system is less than 0.4 mM, wherein the cell-free protein synthesis system does not comprise tryptone, wherein the cell-free protein synthesis system does not comprise yeast extract, and wherein the minimal media comprises at least one of (a) a carbon source; (b) a ammonium source; (c) a magnesium source; (d) a phosphate source; (e) a sulfur source; (f) a trace mineral source; and (g) sodium succinate, wherein the carbon source is chosen from D-glucose and glycerol, wherein the concentration of the D-glucose in the minimal media is from 0.10% to 2.5% (w/v), wherein the concentration of the glycerol in the minimal media is from 0.25% to 25% (w/v), wherein the concentration of the sodium succinate in the minimal media is from 0.05% to 5% (w/v), wherein the phosphate source comprises a sodium phosphate buffer or a potassium phosphate buffer, wherein the concentration of the potassium phosphate buffer in the minimal media is from 5 to 250 mM, wherein the pH of the potassium phosphate buffer in the minimal media is from 6.7 to 7.7, wherein the ammonium source comprises ammonium chloride, wherein the concentration of the ammonium chloride in the minimal media is from 5 to 250 mM, wherein the sulfur source comprises sodium sulfate, wherein the concentration of the sodium sulfate in the minimal media is from 0.5 to 25 mM, wherein the magnesium source comprises magnesium sulfate heptahydrate, wherein the concentration of the magnesium sulfate heptahydrate in the minimal media is from 0.2 to 20 mM, wherein the trace mineral source is chosen from an iron source, a copper source, a nickel source, a zinc source, a molybdenum source, a boron source, and a manganese source, wherein the iron source comprises ferric citrate, wherein the trace mineral source is chosen from an iron source, a copper source, a nickel source, a zinc source, a molybdenum source, a boron source, and a manganese source, wherein the iron source comprises ferric citrate, wherein the concentration of the ferric citrate in the minimal media is from 10 to 1,000 M, wherein the bacteria are Escherichia coli (E. coli), Bacillus subtilis (B. subtilis), or Vibrio natriegens (V. natriegens), wherein the cell-free cell extract is prepared by preparing a suspension in which each one gram of the bacteria is suspended in 1.1 mL of a suspension buffer, wherein the cell-free cell extract is prepared by lysing the bacteria in the suspension, wherein the lysing comprises sonication, wherein the suspension buffer comprises one or more of (a) Tris-acetate; (b) magnesium glutamate; and (c) potassium glutamate, and wherein the concentration of the Tris-acetate in the suspension buffer is from 1 to 100 mM.

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the system produces the protein of interest when maintained at the temperature, wherein the cell-free cell extract is prepared by preparing a suspension in which each one gram of the bacteria is suspended in 1.1 mL of a suspension buffer, wherein the suspension buffer comprises one or more of (a) Tris-acetate; (b) magnesium glutamate; and (c) potassium glutamate, and wherein the concentration of the magnesium glutamate in the suspension buffer is from 1 to 100 mM.

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the system produces the protein of interest when maintained at the temperature, wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5, wherein the temperature is less than about 30° C., wherein the reaction mixture further comprises at least one of: (a) a molecular crowder; (b) a carbon source; (c) a ammonium source; (d) a potassium source; (e) a magnesium source; (f) a phosphate source; and (g) a sulfur source; and (h) a buffer, wherein the molecular crowder, if present, is chosen from PEG 400-8000, maltodextrin, Ficoll 70-400, dextran, and serum albumin, optionally bovine serum albumin, wherein the concentration of the molecular crowder in the cell-free protein synthesis system is from about 0 to 40 g/L, wherein the carbon source, if present, is chosen from glucose, fructose, galactose, sucrose, lactose, gluconate, starch, maltodextrin, and maltose, wherein the concentration of the carbon source, if present, in the cell-free protein synthesis system is from about 0 to 50 g/L, wherein the concentration of the ammonium source, if present, in the cell-free protein synthesis system is from 2 to 20 mM, wherein the potassium source, if present, is chosen from potassium glutamate and potassium gluconate, wherein the concentration of the potassium glutamate in the cell-free protein synthesis system is from about 10 to 270 mM, wherein the concentration of the potassium gluconate in the cell-free protein synthesis system is from about 10 to 250 mM, wherein the concentration of the magnesium source, if present, in the cell-free protein synthesis system is from 0 to about 10 mM, wherein the concentration of the phosphate source, if present, in the cell-free protein synthesis system is from about 5 to about 23.5 mM, wherein the concentration of the sulfur source, if present, in the cell-free protein synthesis system is from 2 to 20 mM, and wherein the concentration of the buffer in the cell-free protein synthesis system is from 50 to 300 mM, wherein the concentration of the cell-free cell extract in the cell-free protein synthesis system is from 10% to 70% (v/v), wherein the concentration of the DNA or RNA encoding the protein of interest in the cell-free protein synthesis system is from 1 to 100 μg/mL, wherein the concentration of free amino acids in the cell-free protein synthesis system is less than 2 mM, wherein the concentration of free NADH/NADPH in the cell-free protein synthesis system is less than 0.4 mM, wherein the cell-free protein synthesis system does not comprise tryptone, wherein the cell-free protein synthesis system does not comprise yeast extract, and wherein the minimal media comprises at least one of (a) a carbon source; (b) a ammonium source; (c) a magnesium source; (d) a phosphate source; (e) a sulfur source; (f) a trace mineral source; and (g) sodium succinate, wherein the carbon source is chosen from D-glucose and glycerol, wherein the concentration of the D-glucose in the minimal media is from 0.10% to 2.5% (w/v), wherein the concentration of the glycerol in the minimal media is from 0.25% to 25% (w/v), wherein the concentration of the sodium succinate in the minimal media is from 0.05% to 5% (w/v), wherein the phosphate source comprises a sodium phosphate buffer or a potassium phosphate buffer, wherein the concentration of the potassium phosphate buffer in the minimal media is from 5 to 250 mM, wherein the pH of the potassium phosphate buffer in the minimal media is from 6.7 to 7.7, wherein the ammonium source comprises ammonium chloride, wherein the concentration of the ammonium chloride in the minimal media is from 5 to 250 mM, wherein the sulfur source comprises sodium sulfate, wherein the concentration of the sodium sulfate in the minimal media is from 0.5 to 25 mM, wherein the magnesium source comprises magnesium sulfate heptahydrate, wherein the concentration of the magnesium sulfate heptahydrate in the minimal media is from 0.2 to 20 mM, wherein the trace mineral source is chosen from an iron source, a copper source, a nickel source, a zinc source, a molybdenum source, a boron source, and a manganese source, wherein the iron source comprises ferric citrate, wherein the concentration of the ferric citrate in the minimal media is from 10 to 1,000 M, wherein the bacteria are Escherichia coli (E. coli), Bacillus subtilis (B. subtilis), or Vibrio natriegens (V. natriegens), wherein the cell-free cell extract is prepared by preparing a suspension in which each one gram of the bacteria is suspended in 1.1 mL of a suspension buffer, wherein the cell-free cell extract is prepared by lysing the bacteria in the suspension, wherein the lysing comprises sonication, wherein the suspension buffer comprises one or more of (a) Tris-acetate; (b) magnesium glutamate; and (c) potassium glutamate, wherein the concentration of the Tris-acetate in the suspension buffer is from 1 to 100 mM, and wherein the concentration of the magnesium glutamate in the suspension buffer is from 1 to 100 mM.

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the system produces the protein of interest when maintained at the temperature, wherein the cell-free cell extract is prepared by preparing a suspension in which each one gram of the bacteria is suspended in 1.1 mL of a suspension buffer, wherein the suspension buffer comprises one or more of (a) Tris-acetate; (b) magnesium glutamate; and (c) potassium glutamate, and wherein wherein the concentration of the potassium glutamate in the suspension buffer is from 1 to 180 mM.

In some embodiments, provided herein is a method for preparing a protein of interest, comprising (1) incubating a reaction mixture comprising DNA or RNA encoding a protein of interest with a a cell-free cell extract comprising supernatant collected from lysed bacteria grown in minimal media, wherein the system does not comprise free amino acids, free nucleotides, or free NADH/NADPH, except when endogenous to the lysed bacteria; and (2) maintaining the system at a temperature of less than about about 30° C., wherein the system produces the protein of interest when maintained at the temperature, wherein the pH of the of the cell-free protein synthesis system is from about 7.0 to about 8.0, about 7.1 to 7.9, about 7.2 to 7.8, about 7.3 to 7.7, about 7.4 to 7.6, or about 7.5, wherein the temperature is less than about 30° C., wherein the reaction mixture further comprises at least one of (a) a molecular crowder; (b) a carbon source; (c) a ammonium source; (d) a potassium source; (e) a magnesium source; (f) a phosphate source; and (g) a sulfur source; and (h) a buffer, wherein the molecular crowder, if present, is chosen from PEG 400-8000, maltodextrin, Ficoll 70-400, dextran, and serum albumin, optionally bovine serum albumin, wherein the concentration of the molecular crowder in the cell-free protein synthesis system is from about 0 to 40 g/L, wherein the carbon source, if present, is chosen from glucose, fructose, galactose, sucrose, lactose, gluconate, starch, maltodextrin, and maltose, wherein the concentration of the carbon source, if present, in the cell-free protein synthesis system is from about 0 to 50 g/L, wherein the concentration of the ammonium source, if present, in the cell-free protein synthesis system is from 2 to 20 mM, wherein the potassium source, if present, is chosen from potassium glutamate and potassium gluconate, wherein the concentration of the potassium glutamate in the cell-free protein synthesis system is from about 10 to 270 mM, wherein the concentration of the potassium gluconate in the cell-free protein synthesis system is from about 10 to 250 mM, wherein the concentration of the magnesium source, if present, in the cell-free protein synthesis system is from 0 to about 10 mM, wherein the concentration of the phosphate source, if present, in the cell-free protein synthesis system is from about 5 to about 23.5 mM, wherein the concentration of the sulfur source, if present, in the cell-free protein synthesis system is from 2 to 20 mM, and wherein the concentration of the buffer in the cell-free protein synthesis system is from 50 to 300 mM, wherein the concentration of the cell-free cell extract in the cell-free protein synthesis system is from 10% to 70% (v/v), wherein the concentration of the DNA or RNA encoding the protein of interest in the cell-free protein synthesis system is from 1 to 100 μg/mL, wherein the concentration of free amino acids in the cell-free protein synthesis system is less than 2 mM, wherein the concentration of free NADH/NADPH in the cell-free protein synthesis system is less than 0.4 mM, wherein the cell-free protein synthesis system does not comprise tryptone, wherein the cell-free protein synthesis system does not comprise yeast extract, and wherein the minimal media comprises at least one of (a) a carbon source; (b) a ammonium source; (c) a magnesium source; (d) a phosphate source; (e) a sulfur source; (f) a trace mineral source; and (g) sodium succinate, wherein the carbon source is chosen from D-glucose and glycerol, wherein the concentration of the D-glucose in the minimal media is from 0.10% to 2.5% (w/v), wherein the concentration of the glycerol in the minimal media is from 0.25% to 25% (w/v), wherein the concentration of the sodium succinate in the minimal media is from 0.05% to 5% (w/v), wherein the phosphate source comprises a sodium phosphate buffer or a potassium phosphate buffer, wherein the concentration of the potassium phosphate buffer in the minimal media is from 5 to 250 mM, wherein the pH of the potassium phosphate buffer in the minimal media is from 6.7 to 7.7, wherein the ammonium source comprises ammonium chloride, wherein the concentration of the ammonium chloride in the minimal media is from 5 to 250 mM, wherein the sulfur source comprises sodium sulfate, wherein the concentration of the sodium sulfate in the minimal media is from 0.5 to 25 mM, wherein the magnesium source comprises magnesium sulfate heptahydrate, wherein the concentration of the magnesium sulfate heptahydrate in the minimal media is from 0.2 to 20 mM, wherein the trace mineral source is chosen from an iron source, a copper source, a nickel source, a zinc source, a molybdenum source, a boron source, and a manganese source, wherein the iron source comprises ferric citrate, wherein the concentration of the ferric citrate in the minimal media is from 10 to 1,000 M, wherein the bacteria are Escherichia coli (E. coli), Bacillus subtilis (B. subtilis), or Vibrio natriegens (V. natriegens), wherein the cell-free cell extract is prepared by preparing a suspension in which each one gram of the bacteria is suspended in 1.1 mL of a suspension buffer, wherein the cell-free cell extract is prepared by lysing the bacteria in the suspension, wherein the lysing comprises sonication, wherein the suspension buffer comprises one or more of (a) Tris-acetate; (b) magnesium glutamate; and (c) potassium glutamate, wherein the concentration of the Tris-acetate in the suspension buffer is from 1 to 100 mM, wherein the concentration of the magnesium glutamate in the suspension buffer is from 1 to 100 mM, and wherein wherein the concentration of the potassium glutamate in the suspension buffer is from 1 to 180 mM.

Further embodiments of the invention are provided in the following numbered paragraphs:
1. A cell-free protein synthesis system, the system comprising:
   a) a cell-free cell extract comprising supernatant collected from lysed bacteria grown in minimal media; and
   b) a reaction mixture comprising a DNA or RNA encoding a protein of interest, wherein the system does not comprise free amino acids, free nucleotides, or free NADH/NADPH, except when endogenous to the lysed bacteria.
2. The system of paragraph 1, wherein the bacteria are E. coli, B. subtilis, or V. natriegens.
3. The system of paragraph 1 or paragraph 2, wherein the pH of the system is from about 7.0 to about 8.0.
4. The system of any one of paragraphs 1-3, wherein the pH of the system is about 7.5.
5. The system of any one of paragraphs 1-4, wherein the system is maintained at a temperature from about 20° C. to about 30° C.
6. The system of any one of paragraphs 1-5, wherein the system is maintained at a temperature of about 25° C.
7. The system of any one of paragraphs 1-6, wherein the reaction mixture further comprises:
   a) a molecular crowder;
   b) a carbon source;
   c) an ammonium source;
   d) a potassium source;
   e) a magnesium source;
   f) a phosphate source; and
   g) a sulfur source; and optionally
   h) a buffer.
8. The system of paragraph 7, wherein the molecular crowder is chosen from PEG 400-8000, maltodextrin, Ficoll 70-400, dextran, and bovine serum albumin.
9. The system of any one of paragraphs 7-8, wherein the molecular crowder comprises PEG 8000.
10. The system of any one of paragraphs 7-9, wherein the concentration of the molecular crowder in the system is from 0 to 40 g/L.
11. The system of any one of paragraphs 7-10, wherein the concentration of the molecular crowder in the system is 20 g/L.
12. The system of any one of paragraphs 7-11, wherein the carbon source in the reaction mixture comprises a sugar.
13. The system of any one of paragraphs 7-12, wherein the carbon source in the reaction mixture comprises maltose.
14. The system of any one of paragraphs 7-13, wherein the carbon source in the reaction mixture does not comprise maltose.
15. The system of any one of paragraphs 7-14 wherein the concentration of the carbon source in the system is from 0 to 50 g/L.
16. The system of any one of paragraphs 7-15, wherein the concentration of the carbon source in the system is 20 g/L.
17. The system of any one of paragraphs 7-16, wherein the ammonium source in the reaction mixture comprises ammonium sulfate.
18. The system of any one of paragraphs 7-17, wherein the concentration of the ammonium source in the system is from 2 to 20 mM.
19. The system of any one of paragraphs 7-18, wherein the concentration of the ammonium source in the system is 12 mM.
20. The system of any one of paragraphs 7-19, wherein the potassium source in the reaction mixture is chosen from potassium glutamate and potassium gluconate.
21. The system of paragraph 20, wherein the concentration of the potassium glutamate in the system is from 10 to 300 mM.
22. The system of paragraph 20 or paragraph 21, wherein the concentration of the potassium glutamate in the system is 20 mM.
23. The system of any one of paragraphs 20-22, wherein the concentration of the potassium gluconate in the system is from 10 to 250 mM.
24. The system of any one of paragraphs 20-23, wherein the concentration of the potassium gluconate in the system is 100 mM.
25. The system of any one of paragraphs 7-24, wherein the magnesium source in the reaction mixture comprises magnesium glutamate.
26. The system of any one of paragraphs 7-25, wherein the concentration of the magnesium source in the system is from 0 to 10 mM.
27. The system of any one of paragraphs 7-26, wherein the concentration of the magnesium source in the system is 4 mM.
28. The system of any one of 7-27, wherein the phosphate source in the reaction mixture comprises potassium phosphate.
29. The system of any one of paragraphs 7-28, wherein the concentration of the phosphate source in the system is from 5 to 25 mM.
30. The system of any one of paragraphs 7-29, wherein the concentration of the phosphate source in the system is 13 mM.
31. The system of any one of paragraphs 7-30, wherein the sulfur source in the reaction mixture comprises ammonium sulfate.
32. The system of any one of paragraphs 7-31, wherein the concentration of the sulfur source in the system is from 2 to 20 mM.
33. The system of any one of paragraphs 7-32, wherein the concentration of the sulfur source in the system is 12 mM.
34. The system of any one of paragraphs 7-33, wherein the system comprises the buffer in the reaction mixture, and wherein the buffer comprises 3-(N-morpholino)propanesulfonic acid (MOPS) buffer.
35. The system of any one of paragraphs 7-34, wherein the system comprises the buffer in the reaction mixture, and wherein the concentration of the buffer in the system is from 50 to 300 mM.
36. The system of any one of paragraphs 7-35, wherein the system comprises the buffer in the reaction mixture, and wherein the concentration of the buffer in the system is 100 mM.
37. The system of any one of the preceding paragraphs, wherein the DNA or RNA is a plasmid.
38. The system of any one of the preceding paragraphs, wherein the concentration of the DNA or RNA in the system is 10 μg/mL.
39. The system of any one of the preceding paragraphs, wherein the concentration of the cell-free cell extract in the system is 30% (v/v).
40. The system of paragraph 39, wherein the cell-free cell extract is prepared by preparing a suspension in which each one gram of the bacteria is suspended in 1.1 mL of a suspension buffer.
41. The system of paragraph 40, wherein the cell-free cell extract is prepared by lysing the bacteria in the suspension.
42. The system of paragraph 41, wherein the lysing comprises sonication.
43. The system of any one of paragraphs 40-42, wherein the suspension buffer comprises
   a) Tris-acetate;
   b) magnesium glutamate; and
   c) potassium glutamate.
44. The system of paragraph 43, wherein the concentration of the Tris-acetate in the suspension buffer is 10 mM.
45. The system of paragraph 43 or paragraph 44, wherein the concentration of the magnesium glutamate in the suspension buffer is 14 mM.
46. The system of any one of paragraphs 43-45, wherein the concentration of the potassium glutamate in the suspension buffer is 60 mM.
47. The system of any one of the preceding paragraphs, comprising free amino acids endogenous to the lysed bacteria, wherein the concentration of free amino acids in the system is less than 1 mM.
48. The system of any one of the preceding paragraphs, comprising free amino acids endogenous to the lysed bacteria, wherein the concentration of the free amino acids in the system is less than 0.5 mM.
49. The system of any one of the preceding paragraphs, comprising free amino acids endogenous to the lysed bacteria, wherein the concentration of the free amino acids in the system is less than 0.1 mM.
50. The system of any one of the preceding paragraphs, comprising free nucleotides endogenous to the lysed bacteria, wherein the concentration of free nucleotides in the system is less than 0.25 mM.
51. The system of any one of the preceding paragraphs, comprising free nucleotides endogenous to the lysed bacteria, wherein the concentration of the free nucleotides in the system is less than 0.125 mM.
52. The system of any one of the preceding paragraphs, comprising free nucleotides endogenous to the lysed bacteria, wherein the concentration of the free nucleotides in the system is less than 0.025 mM.
53. The system of any one of the preceding paragraphs, comprising free NADH/NADPH endogenous to the lysed bacteria, wherein the concentration of free NADH/NADPH in the system is less than 0.2 mM.
54. The system of any one of the preceding paragraphs, comprising free NADH/NADPH endogenous to the lysed bacteria, wherein the concentration of the free NADH/NADPH in the system is less than 0.1 mM.
55. The system of any one of the preceding paragraphs, comprising free NADH/NADPH endogenous to the lysed bacteria, wherein the concentration of the free NADH/NADPH in the system is less than 0.02 mM.
56. The system of any one of the preceding paragraphs, wherein the system does not comprise tryptone.
57. The system of any one of the preceding paragraphs, wherein the system does not comprise yeast extract.
58. The system of any one of the preceding paragraphs, wherein the minimal media comprises:
   a) a carbon source;
   b) a ammonium source;
   c) a magnesium source;
   d) a phosphate source;
   e) a sulfur source; and
   f) a trace mineral source; and optionally
   g) sodium succinate.
59. The system of paragraph 58, wherein the carbon source in the minimal media is chosen from D-glucose and glycerol.
60. The system of paragraph 58 or 59, wherein the concentration of the carbon source in the minimal media is from 0.5% to 4% (w/v).
61. The system of paragraph 59 or paragraph 60, wherein the concentration of the D-glucose in the minimal media is 0.5% (w/v).
62. The system of any one of paragraphs 58-61, wherein the concentration of the glycerol in the minimal media is 2.5% (w/v).
63. The system of any one of paragraphs 58-62, wherein ammonium source in the minimal media comprises ammonium chloride.
64. The system of any one of paragraphs 58-63, wherein the concentration of the ammonium source in the minimal media is 50 mM.
65. The system of any one of paragraphs 58-64, wherein the magnesium source in the minimal media comprises magnesium sulfate heptahydrate.
66. The system of any one of paragraphs 58-65, wherein the concentration of the magnesium source in the minimal media is 2 mM.
67. The system of any one of paragraphs 58-66, wherein the phosphate source in the minimal media comprises a sodium phosphate buffer or a potassium phosphate buffer.
68. The system of any one of paragraphs 58-67, wherein the concentration of the phosphate source in the minimal media is from 25 mM to 150 mM.
69. The system of any one of paragraphs 58-68, wherein the concentration of the phosphate source in the minimal media is 50 mM.
70. The system of any one of paragraphs 67-69, wherein the pH of the sodium phosphate buffer or the potassium phosphate buffer in the minimal media is 7.2.
71. The system of any one of paragraphs 58-70, wherein the sulfur source in the minimal media comprises sodium sulfate.
72. The system of any one of paragraphs 58-71, wherein the concentration of the sulfur source in the minimal media is 5 mM.
73. The system of any one of paragraphs 58-72, wherein the trace mineral source in the minimal media is chosen from an iron source, a copper source, a nickel source, a zinc source, a molybdenum source, a boron source, and a manganese source.
74. The system of paragraph 73, wherein the iron source comprises ferric citrate.
75. The system of paragraph 73 or paragraph 74, wherein the concentration of the iron source in the minimal media is 100 M.
76. The system of any one of paragraphs 58-75, wherein the minimal media comprises sodium succinate, and the concentration of the sodium succinate in the minimal media is 0.375% (w/v).
77. A method for preparing a protein of interest, comprising using the system of any one of the preceding paragraphs.
78. A reaction mixture for use in a cell-free protein synthesis system, the reaction mixture comprising a DNA or RNA encoding a protein of interest and further comprising:
   a) a molecular crowder;
   b) a carbon source;
   c) a ammonium source;
   d) a potassium source;
   e) a magnesium source;
   f) a phosphate source; and
   g) a sulfur source; and optionally
   h) a buffer.
79. The reaction mixture of paragraph 78, wherein the molecular crowder is chosen from PEG 400-8000, maltodextrin, Ficoll 70-400, dextran, and bovine serum albumin.
80. The reaction mixture of paragraph 78 or paragraph 79, wherein the molecular crowder comprises PEG 8000.
81. The reaction mixture of any one of paragraphs 78-80, wherein when the reaction mixture is comprised in a cell-free protein synthesis system, the concentration of the molecular crowder in the system is from 0 to 40 g/L.
82. The reaction mixture of any one of paragraphs 78-81, wherein when the reaction mixture is comprised in a cell-free protein synthesis system, the concentration of the molecular crowder in the system is 20 g/L.
83. The reaction mixture of any one of paragraphs 78-82, wherein the carbon source comprises a sugar.
84. The reaction mixture of any one of paragraphs 78-83, wherein the carbon source comprises maltose.
85. The reaction mixture of any one of paragraphs 78-84, wherein the carbon source does not comprise maltose.
86. The reaction mixture of any one of paragraphs 78-85, wherein when the reaction mixture is comprised in a cell-free protein synthesis system, the concentration of the carbon source in the system is from 0 to 50 g/L.
87. The reaction mixture of any one of paragraphs 78-86, wherein when the reaction mixture is comprised in a cell-free protein synthesis system, the concentration of the carbon source in the system is 20 g/L.
88. The reaction mixture of any one of paragraphs 78-87, wherein the ammonium source comprises ammonium sulfate.
89. The reaction mixture of any one of paragraphs 78-88, wherein when the reaction mixture is comprised in a cell-free protein synthesis system, the concentration of the ammonium source in the system is from 2 to 20 mM.
90. The reaction mixture of any one of paragraphs 78-89, wherein when the reaction mixture is comprised in a cell-free protein synthesis system, the concentration of the ammonium source in the system is 12 mM.
91. The reaction mixture of any one of paragraphs 78-90, wherein the potassium source is chosen from potassium glutamate and potassium gluconate.
92. The reaction mixture of paragraph 91, wherein when the reaction mixture is comprised in a cell-free protein synthesis system, the concentration of the potassium glutamate in the system is from 10 to 300 mM.
93. The reaction mixture of paragraph 91 or 92, wherein when the reaction mixture is comprised in a cell-free protein synthesis system, the concentration of the potassium glutamate in the system is 20 mM.
94. The reaction mixture of any one of paragraphs 91-93, wherein when the reaction mixture is comprised in a cell-free protein synthesis system, the concentration of the potassium gluconate in the system is from 10 to 250 mM.
95. The reaction mixture of any one of paragraphs 91-94, wherein when the reaction mixture is comprised in a cell-free protein synthesis system, the concentration of the potassium gluconate in the system is 100 mM.
96. The reaction mixture of any one of paragraphs 91-95, wherein the magnesium source comprises magnesium glutamate.
97. The reaction mixture of any one of paragraphs 78-96, wherein when the reaction mixture is comprised in a cell-free protein synthesis system, the concentration of the magnesium source in the system is from 0 to 10 mM.
98. The reaction mixture of any one of paragraphs 78-97, wherein when the reaction mixture is comprised in a cell-free protein synthesis system, the concentration of the magnesium source in the system is 4 mM.
99. The reaction mixture of any one of paragraphs 78-98, wherein the phosphate source comprises potassium phosphate.
100. The reaction mixture of any one of paragraphs 78-99, wherein when the reaction mixture is comprised in a cell-free protein synthesis system, the concentration of the phosphate source in the system is from 5 to 25 mM.
101. The reaction mixture of any one of paragraphs 78-100, wherein when the reaction mixture is comprised in a cell-free protein synthesis system, the concentration of the phosphate source in the system is 13 mM.
102. The reaction mixture of any one of paragraphs 78-101, wherein the sulfur source comprises ammonium sulfate.
103. The reaction mixture of any one of paragraphs 78-102, wherein when the reaction mixture is comprised in a cell-free protein synthesis system, the concentration of the sulfur source in the system is from 2 to 20 mM.
104. The reaction mixture of any one of paragraphs 78-103, wherein when the reaction mixture is comprised in a cell-free protein synthesis system, the concentration of the sulfur source in the system is 12 mM.
105. The reaction mixture of any one of paragraphs 78-104, wherein the system comprises the buffer, and wherein the buffer comprises 3-(N-morpholino)propanesulfonic acid (MOPS) buffer.
106. The reaction mixture of any one of paragraphs 78-105, wherein the system comprises the buffer, and wherein when the reaction mixture is comprised in a cell-free protein synthesis system, the concentration of the buffer in the system is from 50 to 300 mM.
107. The reaction mixture of any one of paragraphs 78-106, wherein the system comprises the buffer, and wherein when the reaction mixture is comprised in a cell-free protein synthesis system, the concentration of the buffer in the system is 100 mM.
108. The reaction mixture of any one of paragraphs 78-107, wherein the DNA or RNA is a plasmid.
109. The reaction mixture of any one of paragraphs 78-108, wherein when the reaction mixture is comprised in a cell-free protein synthesis system, the concentration of the DNA or RNA in the system is 10 μg/mL.
110. A method for preparing a protein of interest, comprising
   1) incubating a reaction mixture comprising DNA or RNA encoding a protein of interest with a cell-free cell extract comprising supernatant collected from lysed bacteria grown in minimal media, wherein the system does not comprise free amino acids, free nucleotides, or free NADH/NADPH, except when endogenous to the lysed bacteria; and
   2) maintaining the system at a temperature of less than about 30° C., wherein the system produces the protein of interest when maintained at the temperature.
111. The method of paragraph 110, wherein the method further comprises purifying the protein of interest.
112. The method of paragraph 110 or paragraph 111, wherein the bacteria are E. coli, B. subtilis, or V. natriegens.
113. The method of any one of paragraphs 110-112, wherein the pH of the system is from about 7.0 to about 8.0.
114. The method of any one of paragraphs 110-113, wherein the pH of the system is about 7.5.
115. The method of any one of paragraphs 110-114, wherein the temperature is from about 20° C. to about 30° C.
116. The method of any one of paragraphs 110-115, wherein the temperature is about 25° C.
117. The method of any one of paragraphs 110-116, wherein the reaction mixture further comprises:
   a) a molecular crowder;
   b) a carbon source;
   c) a ammonium source;
   d) a potassium source;
   e) a magnesium source;
   f) a phosphate source; and
   g) a sulfur source; and optionally
   h) a buffer.
118. The method of the immediately preceding paragraph, wherein the molecular crowder in the reaction mixture is chosen from PEG 400-8000, maltodextrin, Ficoll 70-400, dextran, and bovine serum albumin.
119. The method of paragraph 117 or paragraph 118, wherein the molecular crowder in the reaction mixture comprises PEG 8000.
120. The method of any one of paragraphs 117-119, wherein the concentration of the molecular crowder in the system is from 0 to 40 g/L.
121. The method of any one of paragraphs 117-120, wherein the concentration of the molecular crowder in the system is 20 g/L.
122. The method of any one of paragraphs 117-121, wherein the carbon source in the reaction mixture comprises a sugar.
123. The method of any one of paragraphs 117-122, wherein the carbon source in the reaction mixture comprises maltose.
124. The method of any one of paragraphs 117-123, wherein the carbon source in the reaction mixture does not comprise maltose.
125. The method of any one of paragraphs 117-124, wherein the concentration of the carbon source in the system is from 0 to 50 g/L.
126. The method of any one of paragraphs 117-125, wherein the concentration of the carbon source in the system is 20 g/L.
127. The method of any one of paragraphs 117-126, wherein the ammonium source in the reaction mixture comprises ammonium sulfate.
128. The method of any one of paragraphs 117-127, wherein the concentration of the ammonium source in the system is from 2 to 20 mM.
129. The method of any one of paragraphs 117-128, wherein the concentration of the ammonium source in the system is 12 mM.
130. The method of any one of paragraphs 117-129, wherein the potassium source in the reaction mixture is chosen from potassium glutamate and potassium gluconate.
131. The method of paragraph 130, wherein the concentration of the potassium glutamate in the system is from 10 to 300 mM.
132. The method of paragraph 130 or paragraph 131, wherein the concentration of the potassium glutamate in the system is 20 mM.
133. The method of any one of paragraphs 130-132, wherein the concentration of the potassium gluconate in the system is from 10 to 250 mM.
134. The method of any one of paragraphs 130-133, wherein the concentration of the potassium gluconate in the system is 100 mM.
135. The method of any one of paragraphs 130-134, wherein the magnesium source in the reaction mixture comprises magnesium glutamate.
136. The method of any one of paragraphs 117-135, wherein the concentration of the magnesium source in the system is from 0 to 10 mM.
137. The method of any one of paragraphs 117-136, wherein the concentration of the magnesium source in the system is 4 mM.
138. The method of any one of paragraphs 117-137, wherein the phosphate source in the reaction mixture comprises potassium phosphate.
139. The method of any one of paragraphs 117-138, wherein the concentration of the phosphate source in the system is from 5 to 25 mM.
140. The method of any one of paragraphs 117-139, wherein the concentration of the phosphate source in the system is 13 mM.
141. The method of any one of paragraphs 117-140, wherein the sulfur source in the reaction mixture comprises ammonium sulfate.
142. The method of any one of paragraphs 117-141, wherein the concentration of the sulfur source in the system is from 2 to 20 mM.
143. The method of any one of paragraphs 117-142, wherein the concentration of the sulfur source in the system is 12 mM.
144. The method of any one of paragraphs 117-143, wherein the system comprises the buffer in the reaction mixture, and wherein the buffer comprises 3-(N-morpholino)propanesulfonic acid (MOPS) buffer.
145. The method of any one of paragraphs 117-144, wherein the system comprises the buffer in the reaction mixture, and wherein the concentration of the buffer in the system is from 50 to 300 mM.
146. The method of any one of paragraphs 117-145, wherein the system comprises the buffer in the reaction mixture, and wherein the concentration of the buffer in the system is 100 mM.
147. The method of any one of paragraphs 117-146, wherein the DNA or RNA is a plasmid.
148. The method of any one of paragraphs 117-147, wherein the concentration of the DNA or RNA in the system is 10 μg/mL.
149. The method of any one of paragraphs 117-148, wherein the concentration of the cell-free cell extract in the system is 30% (v/v).
150. The method of paragraph 149, wherein the cell-free cell extract is prepared by preparing a suspension in which each one gram of the bacteria is suspended in 1.1 mL of a suspension buffer.
151. The method of paragraph 150, wherein the cell-free cell extract is prepared by lysing the bacteria in the suspension.
152. The method of paragraph 151, wherein the lysing comprises sonication.
153. The method of any one of paragraphs 150-152, wherein the suspension buffer comprises
   a) Tris-acetate;
   b) magnesium glutamate; and
   c) potassium glutamate.
154. The method of paragraph 153, wherein the concentration of the Tris-acetate in the suspension buffer is 10 mM.
155. The method of paragraph 153 or paragraph 154, wherein the concentration of the magnesium glutamate in the suspension buffer is 14 mM.
156. The method of any one of paragraphs 153-155, wherein the concentration of the potassium glutamate in the suspension buffer is 60 mM.
157. The method of any one of paragraphs 110-156, wherein the system comprises free amino acids endogenous to the lysed bacteria, and wherein the concentration of free amino acids in the system is less than 1 mM.
158. The method of any one of paragraphs 110-157, wherein the system comprises free amino acids endogenous to the lysed bacteria, wherein the concentration of the free amino acids in the system is less than 0.5 mM.
159. The method of any one of paragraphs 110-158, wherein the system comprises free amino acids endogenous to the lysed bacteria, and wherein the concentration of the free amino acids in the system is less than 0.1 mM.
160. The method of any one of paragraphs 110-159, wherein the system comprises free nucleotides endogenous to the lysed bacteria, and wherein the concentration of free nucleotides in the system is less than 0.25 mM.
161. The method of any one of paragraphs 110-160, wherein the system comprises free nucleotides endogenous to the lysed bacteria, and wherein the concentration of the free nucleotides in the system is less than 0.125 mM.
162. The method of any one of paragraphs 110-161, wherein the system comprises free nucleotides endogenous to the lysed bacteria, and wherein the concentration of the free nucleotides in the system is less than 0.025 mM.
163. The method of any one of paragraphs 110-162, wherein the system comprises free NADH/NADPH endogenous to the lysed bacteria, and wherein the concentration of free NADH/NADPH in the system is less than 0.2 mM.
164. The method of any one of paragraphs 110-163, wherein the system comprises free NADH/NADPH endogenous to the lysed bacteria, and wherein the concentration of the free NADH/NADPH in the system is less than 0.1 mM.
165. The method of any one of paragraphs 110-164, wherein the system comprises free NADH/NADPH endogenous to the lysed bacteria, and wherein the concentration of the free NADH/NADPH in the system is less than 0.02 mM.
166. The method of any one of paragraphs 110-165, wherein the system does not comprise tryptone.
167. The method of any one of paragraphs 110-166, wherein the system does not comprise yeast extract.
168. The method of any one of the preceding paragraphs, wherein the minimal media comprises:
   a) a carbon source;
   b) a ammonium source;
   c) a magnesium source;
   d) a phosphate source;
   e) a sulfur source; and
   f) a trace mineral source; and optionally
   g) sodium succinate.
169. The method of paragraph 168 wherein the carbon source in the minimal media is chosen from D-glucose and glycerol.
170. The method of paragraph 168 or paragraph 169, wherein the concentration of the carbon source in the minimal media is from 0.5% to 4% (w/v).
171. The method of any one of paragraphs 168-170, wherein the concentration of the D-glucose in the minimal media is 0.5% (w/v).
172. The method of any one of paragraphs 168-171, wherein the concentration of the glycerol in the minimal media is 2.5% (w/v).
173. The method of any one of paragraphs 168-172, wherein the ammonium source in the minimal media comprises ammonium chloride.
174. The method of any one of paragraphs 168-173, wherein the concentration of the ammonium source in the minimal media is 50 mM.
175. The method of any one of paragraphs 168-174, wherein the magnesium source in the minimal media comprises magnesium sulfate heptahydrate.
176. The method of any one of paragraphs 168-175, wherein the concentration of the magnesium source in the minimal media is 2 mM.
177. The method of any one of paragraphs 168-176, wherein the phosphate source in the minimal media comprises a sodium phosphate buffer or a potassium phosphate buffer.
178. The method of any one of paragraphs 168-177, wherein the concentration of the phosphate source in the minimal media is from 25 mM to 150 mM.
179. The method of any one of paragraphs 168-178, wherein the concentration of the phosphate source in the minimal media is 50 mM.
180. The method of any one of paragraphs 168-179, wherein the pH of the sodium phosphate buffer or the potassium phosphate buffer in the minimal media is 7.2.
181. The method of any one of paragraphs 168-180, wherein the sulfur source in the minimal media comprises sodium sulfate.
182. The method of any one of paragraphs 168-181, wherein the concentration of the sulfur source in the minimal media is 5 mM.
183. The method of any one of paragraphs 168-182, wherein the trace mineral source in the minimal media is chosen from an iron source, a copper source, a nickel source, a zinc source, a molybdenum source, a boron source, and a manganese source.
184. The method of paragraph 183, wherein the iron source comprises ferric citrate.
185. The method of paragraph 183 or paragraph 184, wherein the concentration of the iron source in the minimal media is 100 M.
186. The method of any one of paragraphs 168-185, wherein the minimal media comprises sodium succinate, and the concentration of the sodium succinate in the minimal media is 0.375% (w/v).
187. The method, system, buffer or mixture of any of paragraphs 1-187, wherein the bacteria are V. natriegens, and (i) the minimal media contains NaCl at 100-500 mM, optionally 250 mM; and/or the reaction buffer comprises NaCl at 50-250 mM, optionally 200-200 mM, optionally 100 mM.

### EXAMPLES

These examples are provided for illustrative purposes only and not to limit the scope of the claims provided herein.

### Example 1. Preparation of Minimal Cell-Free Cell Extract

E. coli BL21 strain, as well as chemicals for media and reaction mixture were purchased from Sigma-Aldrich (St. Louis, MO). EGFP cloned into pKAN vector using NdeI and XhoI, purchased from Twist Biosciences, was used as a model protein in this study.

E. coli B121 (DE3) cells frozen in 40% glycerol, 60% LB were grown overnight in LB media. The cells were then resuspended in 50 mL of custom synthetic media at 4% (v/v) containing 12.5 μg/mL chloramphenicol and 50 μg/mL IPTG, the chloramphenicol being optional. The synthetic media composition consisted of: 0.5% (w/v) D-glucose, 2.5% (w/v) glycerol, 0.375% (w/v) sodium succinate, 50 mM pH 7.2 potassium phosphate buffer, 50 mM ammonium chloride, 5 mM sodium sulfate, 2 mM magnesium sulfate heptahydrate, and 100 M of ferric citrate. The cells were cultured at 37° C. in an incubator with an RPM of 260 in a 250 mL baffled shake flask. The cultured cells were monitored using a Biotek X plate reader until an OD 600 of 7-10 was reached. The cells were then pelleted at 3,400 RCF at 4° C. for 15-25 minutes, and the supernatant was disposed of. The pelleted cells were then stored overnight at −80° C. or immediately processed. The cells were washed once with cold buffer at a ratio of 20 mL buffer per gram of cells. Buffer A contained 10 mM Tris-acetate (pH 7.9), 14 mM magnesium glutamate, and 60 mM potassium glutamate. The cells were then pelleted and resuspended in buffer at a ratio of 1.1 mL buffer per gram of cells to yield a cell-buffer suspension. 0.5 mL of the cell-buffer suspension was then transferred to a 1.5 mL tube and immersed in an ice-water bath for lysis.

The cells were lysed using a Q125 Sonicator (Qsonica, Newton, CT) with a 3.175 mm diameter probe, a 10 second on, 10 second off cycle, and 50% amplitude. The input energy (Joules) was recorded, and sonication was performed until ˜3,500 J was reached. The input energy was determined by the sonication of E. coli carrying EGFP until none of the pelleted cells appeared green. The cell lysate was then centrifuged at 12,000 RCF at 4° C. for 15 minutes. The supernatant was collected and immediately frozen at −80° C.

### Example 2. Cell-Free Protein Synthesis

### Materials & Methods

The minimal CFPS reactions were carried out in batch format at 10 μL volume using a 1.5 mL Eppendorf tube. The minimal reaction mixture for CFPS consisted of the following components in a final volume of 10 L: 30% (v/v) cell-free cell extract, 10 μg/mL plasmid, 20 mM potassium glutamate, 20 g/L maltose, 12 mM ammonium sulfate, 100 mM MOPS buffer (pH 7.5), 4 mM magnesium glutamate, 100 mM potassium gluconate, 20 g/L polyethylene glycol 8000 (PEG 8000), and 13 mM potassium phosphate (pH 7.2). For effect of component experiments, the control buffer consisted of the above formulation, and each condition was the same minus the stated component that was removed. For commercial CFPS, extracts and mixtures were procured from New England Biolabs under the brand name NEBExpress®. The commercial mixture consisted of a 2× protein synthesis buffer, S30 cell extract, T7 RNA polymerase, and RNase inhibitor. The commercial cell extracts were generated utilizing rich media, and the reaction buffer contained added amino acids and NTPs.

Four combinations of mixture were tested with the commercial 2× buffer used with the minimal cell extract and vice versa. For the minimal cell extracts, only the commercial 2× buffer was utilized, as the T7 polymerase was already present in the cell extracts.

### Results—Effect of Mixture Composition

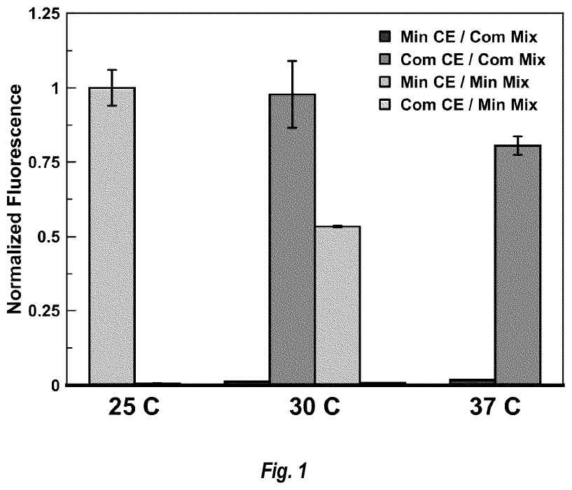

FIG. 1 shows the effects of extract and mixture conditions, as well as temperature, on CFPS deGFP protein yield. “Min” and “Com” refer to “minimal” and “commercial,” respectively, and “CE” refers to cell extract.

The temperature and mixture composition experiment produced surprising results. The minimal mixture and cell extract generated similar yield to the commercial system, but at different temperatures. The minimal system performed best at ˜25° C., with a 50% drop in performance at 30° C. This indicates that at higher temperatures, there may have been a faster breakdown of proteins, leading to an overall drop in reaction lifetime. These results are contrary to conventional knowledge for CFPS, where maximal protein production rates are favored since the pool of feedstock is limited. This conventional knowledge was manifested in the commercial cell-free system, where higher yield is achieved at ˜30° C.

The commercial and minimal cell-free mixtures appeared to be mutually exclusive in performance when commercial extract or mixture is combined with minimal mixture or extract. That is, in FIG. 1, both the combination of the commercial mixture and the minimal cell-free cell extract and the combination of the minimal reaction mixture described above and the commercial extract produced very low yields of EGFP (and therefore low measured fluorescence). These results show that for optimal performance, the reaction buffer/mixture may need to be tailored to the process in which the cell extracts are generated.

### Results—Effect of Individual Reaction Buffer Components

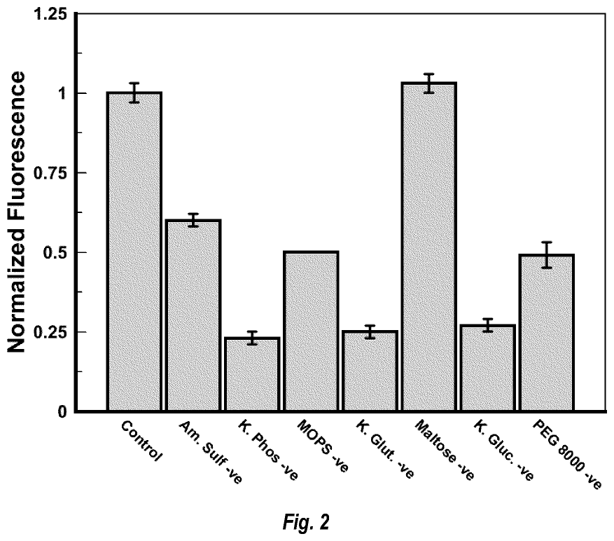

FIG. 2 shows the effect of removing individual reaction mixture components on CFPS deGFP protein yield when utilizing a minimal cell extract. The removal of any reaction component from the minimal reaction buffer resulted in a significant (40%+) drop in performance of CFPS. The only exception is with the removal of maltose, where a slight (+3%) increase in yield was observed. Other than maltose, the following components provide for optimal performance of a minimal CFPS system: Potassium, phosphates, nitrogen, carbons, sulfur, a buffer, and a molecular crowder.

### Example 3: CFPS from Vibrio natriegens

Conditions for culture and production of cell-free protein synthesis systems from V. natriegens were investigated.

### Primary and Secondary Carbon Source

The effect of different primary and secondary carbon sources on growth are shown in Tables 1 and 2 respectively.

TABLE 1

| | OD600 | Doubling time |
|---|---|---|
| Carbon source | (saturation) | (min) |
| Sucrose (2% w/v) | 3.40 | 25 |
| Glucose (2%) | 3.88 | 26 |
| Glycerol (2%) | 9.42 | 46 |

TABLE 2

| | OD600 |
|---|---|
| Carbon source | (saturation) |
| Glycerol | 3.40 |
| Glycerol + succinate (0.5%) | 3.88 |
| Glycerol + gluconate (0.5%) | 9.42 |

The medium composition selected for culture comprised: sodium chloride 250 mM, potassium phosphate 50 mM, ammonium sulfate 25 mM, glycerol 2% w/v, sodium succinate w/v, magnesium chloride 2 mM, ferric citrate 0.2 mM, citric acid 5 mM; calcium 90 microM, manganese 50 microM, zinc 10 microM, nickel 10 microM, copper 10 microM.

### Reaction Buffer

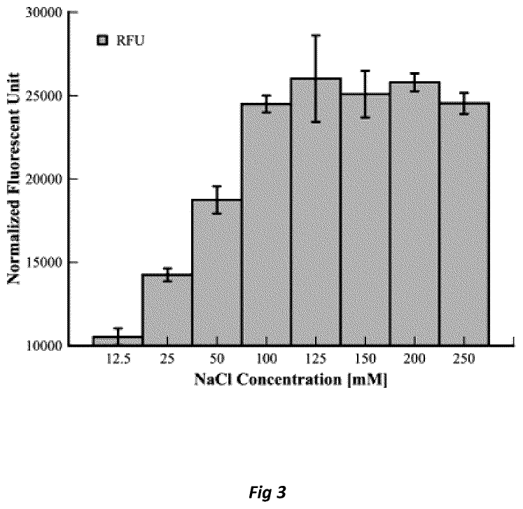

The presence of sodium chloride was found to greatly improve the CFPS reaction for V. natriegens. Saturation occurred at around 100 mM NaCl, but levels up to at least 200 mM were tolerated (FIG. 3). Removal of MOPs and PEG appeared to improve performance, while the optimal pH was 8.0.

Although the foregoing invention has been described in some detail by way of illustration and example for purposes of clarity of understanding, the descriptions and examples should not be construed as limiting the scope of the invention. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby. The disclosures of all patent and scientific literature cited herein are expressly incorporated herein in their entirety by reference. To the extent any material incorporated herein by reference is inconsistent with the express content of this disclosure, the express content controls.

### REFERENCES

Iskakova et al., “Troubleshooting coupled in vitro transcription-translation system derived from Escherichia coli cells: synthesis of high-yield fully active proteins,” Nucleic Acids Research, 34(19): e135 (2006) doi: 10.1093/nar/gkl462.
Li et al, “The metabolic potential of Escherichia coli BL21 in defined and rich medium,” Microb. Cell Fact. 13:45 (2014), https://doi.org/10.1186/1475-2859-13-45.

What is claimed is:

1. A cell-free protein synthesis system, the system comprising:
a) a cell-free cell extract comprising supernatant collected from lysed bacteria grown in minimal media; and
b) a reaction mixture comprising a DNA or RNA encoding a protein of interest,
wherein the system does not comprise free amino acids, free nucleotides, or free NADH/NADPH, except when endogenous to the lysed bacteria.

2. The system of claim 1, wherein
i. the bacteria are E. coli, B. subtilis, or V. natriegens; and/or
ii. the pH of the system is from about 7.0 to about 8.0, optionally 7.5; and/or
iii. the system is maintained at a temperature from about 20° C. to about 30° C., optionally about 25° C.

3. The system of claim 1 or claim 2, wherein the reaction mixture further comprises:
a) a molecular crowder;
b) a carbon source;
c) an ammonium source;
d) a potassium source;
e) a magnesium source;
f) a phosphate source; and
g) a sulfur source; and optionally
h) a buffer.

4. The system of claim 3, wherein
i. the molecular crowder is chosen from PEG 400-8000, maltodextrin, Ficoll 70-400, dextran, and bovine serum albumin, optionally PEG 8000; and/or
ii. the concentration of the molecular crowder in the system is from 0 to 40 g/L, optionally 20 g/L; and/or
iii. the carbon source in the reaction mixture comprises a sugar; and/or
iv. the carbon source in the reaction mixture comprises maltose; and or
v. the carbon source in the reaction mixture does not comprise maltose; and/or
vi. the concentration of the carbon source in the system is from 0 to 50 g/L, optionally 20 g/L; and/or
vii. the ammonium source in the reaction mixture comprises ammonium sulfate; and/or
viii. the concentration of the ammonium source in the system is from 2 to 20 mM, optionally 12 mM; and/or
ix. the potassium source in the reaction mixture is chosen from potassium glutamate and potassium gluconate; and/or
x. the potassium source in the reaction mixture is potassium glutamate and the concentration of the potassium glutamate in the system is from 10 to 300 mM, optionally 20 mM; and/or
xi. the potassium source in the reaction mixture is potassium gluconate and the concentration of the potassium gluconate in the system is from 10 to 250 mM, optionally 100 mM; and/or
xii. the magnesium source in the reaction mixture comprises magnesium glutamate; and/or
xiii. the concentration of the magnesium source in the system is from 0 to 10 mM, optionally 4 mM; and/or
xiv. the phosphate source in the reaction mixture comprises potassium phosphate; and/or
xv. the concentration of the phosphate source in the system is from 5 to 25 mM, optionally 13 mM; and/or
xvi. the sulfur source in the reaction mixture comprises ammonium sulfate; and/or
xvii. the concentration of the sulfur source in the system is from 2 to 20 mM, optionally 12 mM; and/or
xviii. the system comprises the buffer in the reaction mixture, wherein the buffer comprises 3-(N-morpholino)propanesulfonic acid (MOPS) buffer; and/or
xix. wherein the concentration of the buffer in the system is from 50 to 300 mM, optionally 100 mM.

5. The system of any one of the preceding claims, wherein the minimal media comprises:
a) a carbon source;
b) a ammonium source;
c) a magnesium source;
d) a phosphate source;
e) a sulfur source; and
f) a trace mineral source; and optionally
g) sodium succinate;
wherein optionally
i. the carbon source in the minimal media is chosen from D-glucose and glycerol; and/or
ii. the concentration of the carbon source in the minimal media is from 0.5% to 4% (w/v); and/or
iii. the concentration of the glycerol in the minimal media is 2.5% (w/v); and/or
iv. the ammonium source in the minimal media comprises ammonium chloride; and/or
v. the concentration of the ammonium source in the minimal media is 50 mM; and/or
vi. the magnesium source in the minimal media comprises magnesium sulfate heptahydrate; and/or
vii. the concentration of the magnesium source in the minimal media is 2 mM; and/or
viii. the phosphate source in the minimal media comprises a sodium phosphate buffer or a potassium phosphate buffer; and/or
ix. the concentration of the phosphate source in the minimal media is from 25 mM to 150 mM; and/or
x. the concentration of the phosphate source in the minimal media is 50 mM; and/or
xi. The pH of the the sodium phosphate buffer or the potassium phosphate buffer in the minimal media is 7.2; and/or
xii. the sulfur source in the minimal media comprises sodium sulfate; and/or
xiii. the concentration of the sulfur source in the minimal media is 5 mM; and/or
xiv. the trace mineral source in the minimal media is chosen from an iron source, a copper source, a nickel source, a zinc source, a molybdenum source, a boron source, and a manganese source; and/or
xv. the iron source comprises ferric citrate; and/or
xvi. the concentration of the iron source in the minimal media is 100 μM; and/or
xvii. the minimal media comprises sodium succinate, and the concentration of the sodium succinate in the minimal media is 0.375% (w/v).

6. A method for preparing a protein of interest, comprising using the system of any one of the preceding claims.

7. A reaction mixture for use in a cell-free protein synthesis system, the reaction mixture comprising a DNA or RNA encoding a protein of interest and further comprising:
a) a molecular crowder;
b) a carbon source;
c) a ammonium source;
d) a potassium source;
e) a magnesium source;
f) a phosphate source; and
g) a sulfur source; and optionally
h) a buffer; and optionally
i) sodium chloride
wherein optionally one or more of components (a)-(h) are as defined in claim 4.

8. A method for preparing a protein of interest, comprising
1) incubating a reaction mixture comprising DNA or RNA encoding a protein of interest with a cell-free cell extract comprising supernatant collected from lysed bacteria grown in minimal media, wherein the system does not comprise free amino acids, free nucleotides, or free NADH/NADPH, except when endogenous to the lysed bacteria; and
2) maintaining the system at a temperature of less than about 30° C., wherein the system produces the protein of interest when maintained at the temperature.

9. The method of claim 8, wherein the method further comprises purifying the protein of interest.

10. The method of claim 8 or claim 9, wherein the bacteria are E. coli, B. subtilis, or V. natriegens.

11. The method of any one of claims 8-10, wherein the reaction mixture further comprises:
a) a molecular crowder;
b) a carbon source;
c) a ammonium source;
d) a potassium source;
e) a magnesium source;
f) a phosphate source; and
g) a sulfur source; and optionally
h) a buffer;
wherein optionally one or more of components (a)-(h) are as defined in claim 4.
